(19) United States
(12) Patent Application Publication
(10) Pub. No.: US 2009/0099160 A1
(43) Pub. Date: Apr. 16, 2009

(54) 4-(4-(Imidazol-4-Yl) Pyrimidin-2-Ylamino) Benzamides as CDK Inhibitors

(76) Inventors: David Andrews, Cheshire (GB); Maurice Raymond Finlay, Cheshire (GB); Clive Green, Cheshire (GB); Clifford Jones, Cheshire (GB)

Correspondence Address: MORGAN LEWIS & BOCKIUS LLP, 1111 PENNSYLVANIA AVENUE NW, WASHINGTON, DC, 20004, US

(21) Appl. No.: 11/793,254

(22) PCT Filed: Dec. 15, 2005
(86) PCT No.: PCT/GB05/04865

§ 371 Date: May 12, 2008

(30) Foreign Application Priority Data
Dec. 17, 2004 (GB) 0427699.4
Jul. 30, 2005 (GB) 0515740.9

(51) Int. Cl. A61K 31/506; C07D 403/04; C07D 401/14; C07D 405/14; C07D 413/14; A61K 31/541; A61K 31/5377; C07D 417/14; C07D 409/14; A61P 35/00

(52) U.S. Cl. 514/227.8; 544/331; 544/122; 544/58.6; 514/275; 514/235.8

(57) ABSTRACT

Compounds of the formula (I):

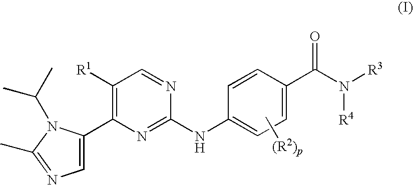

wherein variable groups are as defined within and a pharmaceutically acceptable salts and in vivo hydrolysable esters are described. Also described are processes for their preparation and their use as medicaments, particularly medicaments for producing a cell cycle inhibitory (anti-cell-proliferation) effect in a warm-blooded animal, such as man.

The invention relates to pyrimidine derivatives, or pharmaceutically acceptable salts or in vivo hydrolysable esters thereof, which possess cell-cycle inhibitory activity and are accordingly useful for their anti-cell-proliferation (such as anti-cancer) activity and are therefore useful in methods of treatment of the human or animal body. The invention also relates to processes for the manufacture of said pyrimidine derivatives, to pharmaceutical compositions containing them and to their use in the manufacture of medicaments of use in the production of an anti-cell-proliferation effect in a warm-blooded animal such as man.

The cell cycle is fundamental to the survival, regulation and proliferation of cells and is highly regulated to ensure that each step progresses in a timely and orderly manner. The progression of cells through the cell cycle arises from the sequential activation and de-activation of several members of the cyclin-dependent kinase (CDK) family. The activation of CDKs is dependent on their interaction with a family of intracellular proteins called cyclins. Cyclins bind to CDKs and this association is essential for CDK activity (such as CDK1, CDK2, CDK4 and/or CDK6) within the cell. Different cyclins are expressed and degraded at different points in the cell cycle to ensure that activation and inactivation of CDKs occurs in the correct order for progression through the cell cycle.

Moreover, CDKs appear to be downstream of a number of oncogene signalling pathways. Deregulation of CDK activity by upregulation of cyclins and/or deletion of endogenous inhibitors appears to be an important axis between mitogenic signalling pathways and proliferation of tumour cells.

Accordingly it has been recognised that an inhibitor of cell cycle kinases, particularly inhibitors of CDK1, CDK2 and/or CDK4 (which operate at the G2/M, G1/S-S-G2/M and G1-S phases respectively) should be of value as an active inhibitor of cell proliferation, such as growth of mammalian cancer cells.

The inhibition of cell cycle kinases is expected to be of value in the treatment of disease states associated with aberrant cell cycles and cell proliferation such as cancers (solid tumours and leukemias), fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation.

WO 02/20512, WO 03/076435, WO 03/076436, WO 03/076434, WO 03/076433 and WO 04/101549 describe certain 2-anilino-4-imidazolylpyrimidine derivatives that inhibit the effect of cell cycle kinases. The present invention is based on the discovery that a novel group of 2-(4-amidoanilino)-4-(imidazolyl)pyrimidines inhibit the effects of cell cycle kinases, particularly CDK2, and thus possess anti-cell-proliferation properties. The compounds of the present invention are not specifically disclosed in any of the above applications and we have surprisingly found that these compounds possess beneficial properties in terms of one or more of their pharmacological activity (particularly as compounds which inhibit CDK2) and/or pharmacokinetic, efficacious, metabolic and toxicological profiles that make them particularly suitable for in vivo administration to a warm blooded animal, such as man. In particular these compounds have very high levels of cell and enzyme potency and high levels of exposure in vivo.

Accordingly, the present invention provides a compound of formula (I):

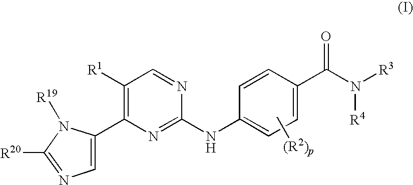

wherein:

R¹ is hydrogen or halo;

R² is halo, nitro, cyano, hydroxy, amino, carboxy, carbamoyl, mercapto, methylthio, mesyl, trifluoromethyl, trifluoromethoxy, C₁₋₆alkyl, C₁₋₆alkoxy, C₂₋₆alkenyl or C₂₋₆alkynyl;

p is 0-4; wherein the values of R² may be the same or different;

R³ and R⁴ are independently selected from hydrogen, C₁₋₆alkyl, C₂₋₆alkenyl, C₂₋₆alkynyl, carbocyclyl or heterocyclyl; wherein R³ and R⁴ may be independently optionally substituted on carbon by one or more R⁵; and wherein if said heterocyclyl contains an —NH— moiety that nitrogen may be optionally substituted by a group selected from R⁶;

R¹⁹ is selected from ethyl, propyl, isopropyl, butyl, iso-butyl, sec-butyl, t-butyl, cyclopropyl, cyclopropylmethyl, 1-cyclopropylethyl or cyclobutyl; wherein R¹ may be optionally substituted on carbon by one or more R²¹;

R²⁰ is methyl, ethyl, isopropyl, fluoromethyl, difluoromethyl, trifluoromethyl, methoxymethyl, cyclopropylmethyl or cyclopropyl;

R⁵ is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl, heterocyclyl, carbocyclylC₁₋₆alkyl-R⁷—, heterocyclylC₁₋₆alkyl-R⁸—, carbocyclyl-R⁹— or heterocyclyl-R¹⁰—; wherein R⁵ may be optionally substituted on carbon by one or more R¹¹; and wherein if said heterocyclyl contains an —NH— moiety that nitrogen may be optionally substituted by a group selected from R¹²;

R⁶ and R¹² are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl; wherein R⁶ and R¹² may be independently optionally substituted on carbon by one or more R¹³;

R⁷, R⁸, R⁹ and R¹⁰ are independently selected from —O—, —N(R¹⁴)—, —C(O)—, —N(R¹⁵)C(O)—, —C(O)N(R¹⁶)—, —S(O)ₛ—, —SO₂N(R¹⁷)— or —N(R¹⁸)SO₂—; wherein R¹⁴, R¹⁵, R¹⁶, R¹⁷ and R¹⁸ are independently selected from hydrogen or C₁₋₆alkyl and s is 0-2;

R¹¹ and R¹³ are independently selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl; and

R²¹ is selected from halo, methoxy and hydroxy;

or a pharmaceutically acceptable salt or an in vivo hydrolysable ester thereof.

Accordingly, the present invention provides a compound of formula (I) which is a compound of formula (Ia):

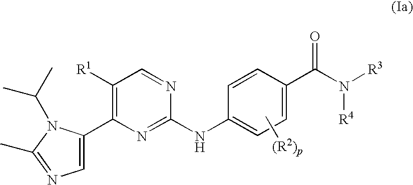

wherein:

R¹ is hydrogen or fluoro;

R² is halo, nitro, cyano, hydroxy, amino, carboxy, carbamoyl, mercapto, C₁₋₆alkyl, C₁₋₆alkoxy, C₂₋₆alkenyl or C₂₋₆alkynyl;

p is 0-4; wherein the values of R² may be the same or different;

R³ and R⁴ are independently selected from hydrogen, C₁₋₆alkyl, C₂₋₆alkenyl, C₂₋₆alkynyl, carbocyclyl or heterocyclyl; wherein R³ and R⁴ may be independently optionally substituted on carbon by one or more R⁵; and wherein if said heterocyclyl contains an —NH— moiety that nitrogen may be optionally substituted by a group selected from R⁶;

R⁵ is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl, heterocyclyl, carbocyclylC₁₋₆alkyl-R⁷—, heterocyclylC₁₋₆alkyl-R⁸—, carbocyclyl-R⁹— or heterocyclyl-R¹⁰—; wherein R⁵ may be optionally substituted on carbon by one or more R¹¹; and wherein if said heterocyclyl contains an —NH— moiety that nitrogen may be optionally substituted by a group selected from R¹²;

R⁶ and R¹² are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl; wherein R⁶ and R¹² may be independently optionally substituted on carbon by one or more R¹³;

R⁷, R⁸, R⁹ and R¹⁰ are independently selected from —O—, —N(R¹⁴)—, —C(O)—, —N(R¹⁵)C(O)—, —C(O)N(R¹⁶)—, —S(O)ₛ—, —SO₂N(R¹⁷)— or —N(R¹⁸)SO₂—; wherein R¹⁴, R¹⁵, R⁶, R¹⁷ and R¹⁸ are independently selected from hydrogen or C₁₋₆alkyl and s is 0-2; and

R¹¹ and R¹³ are independently selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt or an in vivo hydrolysable ester thereof.

In this specification the term “alkyl” includes both straight and branched chain alkyl groups but references to individual alkyl groups such as “propyl” are specific for the straight chain version only. For example, “C₁₋₆alkyl” and “C₁₋₄alkyl” include methyl, ethyl, propyl, isopropyl and τ-butyl. However, references to individual alkyl groups such as ‘propyl’ are specific for the straight chained version only and references to individual branched chain alkyl groups such as ‘isopropyl’ are specific for the branched chain version only. A similar convention applies to other radicals.

Where optional substituents are chosen from “one or more” groups it is to be understood that this definition includes all substituents being chosen from one of the specified groups or the substituents being chosen from two or more of the specified groups.

A “heterocyclyl” is a saturated, partially saturated or unsaturated, mono or bicyclic ring containing 4-12 atoms of which at least one atom is chosen from nitrogen, sulphur or oxygen, which may, unless otherwise specified, be carbon or nitrogen linked, wherein a —CH₂— group can optionally be replaced by a —C(O)—, a ring nitrogen atom may optionally bear a C₁₋₆alkyl group and form a quaternary compound or a ring nitrogen and/or sulphur atom may be optionally oxidised to form the N-oxide and or the S-oxides. Examples and suitable values of the term “heterocyclyl” are morpholino, piperidyl, pyridyl, pyranyl, pyrrolyl, isothiazolyl, indolyl, quinolyl, thienyl, 1,3-benzodioxolyl, thiadiazolyl, piperazinyl, thiazolidinyl, pyrrolidinyl, thiomorpholino, pyrrolinyl, homopiperazinyl, 3,5-dioxapiperidinyl, tetrahydropyranyl, imidazolyl, pyrimidyl, pyrazinyl, pyridazinyl, isoxazolyl, N-methylpyrrolyl, 4-pyridone, 1-isoquinolone, 2-pyrrolidone, 4-thiazolidone, pyridine-N-oxide and quinoline-N-oxide. In one aspect of the invention a “heterocyclyl” is a saturated, partially saturated or unsaturated, mono or bicyclic ring containing 5 or 6 atoms of which at least one atom is chosen from nitrogen, sulphur or oxygen, it may, unless otherwise specified, be carbon or nitrogen linked, a —CH₂— group can optionally be replaced by a —C(O)— and a ring sulphur atom may be optionally oxidised to form the S-oxides.

A “carbocyclyl” is a saturated, partially saturated or unsaturated, mono or bicyclic carbon ring that contains 3-12 atoms; wherein a —CH₂— group can optionally be replaced by a —C(O)—. Particularly “carbocyclyl” is a monocyclic ring containing 5 or 6 atoms or a bicyclic ring containing 9 or 10 atoms. Suitable values for “carbocyclyl” include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl.

An example of “C₁₋₆alkanoyloxy” is acetoxy. Examples of “C₁₋₆alkoxycarbonyl” include methoxycarbonyl, ethoxycarbonyl, n- and t-butoxycarbonyl. Examples of “C₁₋₆alkoxy” include methoxy, ethoxy and propoxy. Examples of “C₁₋₆alkanoylamino” include formamido, acetamido and propionylamino. Examples of “C₁₋₆alkylS(O)ₐ wherein a is 0 to 2” include methylthio, ethylthio, methylsulphinyl, ethylsulphinyl, mesyl and ethylsulphonyl. Examples of “C₁₋₆alkanoyl” include propionyl and acetyl. Examples of “N—(C₁₋₆alkyl)amino” include methylamino and ethylamino. Examples of “N,N—(C₁₋₆alkyl)₂amino” include di-N-methylamino, di-(N-ethyl)amino and N-ethyl-N-methylamino. Examples of “C₂₋₆alkenyl” are vinyl, allyl and 1-propenyl. Examples of “C₂₋₆alkynyl” are ethynyl, 1-propynyl and 2-propynyl. Examples of “N—(C₁₋₆alkyl)sulphamoyl” are N-(methyl)sulphamoyl and N—(ethyl)sulphamoyl. Examples of “N,N—(C₁₋₆alkyl)₂sulphamoyl” are N,N-(dimethyl)sulphamoyl and N-(methyl)-N-(ethyl)sulphamoyl. Examples of “N—(C₁₋₆alkyl)carbamoyl” are methylaminocarbonyl and ethylaminocarbonyl. Examples of “N,N—(C₁₋₆alkyl)₂carbamoyl” are dimethylaminocarbonyl and methylethylaminocarbonyl. Examples of “C₁₋₆alkylsulphonylamino” include methylsulphonylamino, isopropylsulphonylamino and τ-butylsulphonylamino. Examples of “C₁₋₆alkylsulphonyl” include methylsulphonyl, isopropylsulphonyl and t-butylsulphonyl. Examples of “carbocyclylC₁₋₆alkyl-R⁷—” include 1-(carbocyclyl)ethyl-R⁷—, for example 1-(cyclopropyl)ethyl-R⁷— and 1-phenylethyl-R⁷—, and 3-(carbocyclyl)propyl-R⁷—, for example 3-(cyclopentyl)propyl-R⁷— and 3-(naphthyl)propyl-R⁷—. Examples of “heterocyclylC₁₋₆alkyl-R⁸—” include 1-(heterocyclyl)ethyl-R⁸—, for example 1-(pyrid-2-yl)ethyl-R⁸— and 1-(morpholino)ethyl-R⁸—, and 3-(heterocyclyl)propyl-R⁸—, for example 3-(piperazin-1-yl)propyl-R⁸— and 3-(pyrrolidin-1-yl)propyl-R⁸—.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

An in vivo hydrolysable ester of a compound of the formula (I) containing carboxy or hydroxy group is, for example, a pharmaceutically acceptable ester which is hydrolysed in the human or animal body to produce the parent acid or alcohol. Suitable pharmaceutically acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkoxycarbonyloxyC₁₋₆alkyl esters for example 1-cyclohexylcarbonyloxyethyl; 1,3-dioxolen-2-onylmethyl esters for example 5-methyl-1,3-dioxolen-2-onylmethyl; and C₁₋₆alkoxycarbonyloxyethyl esters for example 1-methoxycarbonyloxyethyl and may be formed at any carboxy group in the compounds of this invention.

An in vivo hydrolysable ester of a compound of the formula (I) containing a hydroxy group includes inorganic esters such as phosphate esters and α-acyloxyalkyl ethers and related compounds which as a result of the in vivo hydrolysis of the ester breakdown to give the parent hydroxy group. Examples of α-acyloxyalkyl ethers include acetoxymethoxy and 2,2-dimethylpropionyloxy-methoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. Examples of substituents on benzoyl include morpholino and piperazino linked from a ring nitrogen atom via a methylene group to the 3- or 4-position of the benzoyl ring.

Some compounds of the formula (I) may have chiral centres and/or geometric isomeric centres (E- and Z-isomers), and it is to be understood that the invention encompasses all such optical, diastereoisomers and geometric isomers that possess CDK inhibitory activity.

The invention relates to any and all tautomeric forms of the compounds of the formula (I) that possess CDK inhibitory activity.

It is also to be understood that certain compounds of the formula (I) can exist in solvated as well as unsolvated forms such as, for example, hydrated forms. It is to be understood that the invention encompasses all such solvated forms which possess CDK inhibitory activity.

Particular values of variable groups are as follows. Such values may be used where appropriate with any of the definitions, claims or embodiments defined hereinbefore or hereinafter.

R¹ is hydrogen or fluoro.

R¹ is hydrogen.

R¹ is fluoro.

R² is halo, cyano or C₁₋₆alkyl.

R² is halo or C₁₋₆alkyl.

R² is fluoro, chloro, cyano or methyl.

R² is fluoro, chloro or methyl.

R² is fluoro.

R² is chloro.

R² is cyano.

R² is methyl.

p is 0 or 1.

p is 0.

p is 1.

p is 0-1 and where p is 1, R² is ortho to the —C(O)NR³R⁴ group of formula (I).

p is 1 and R² is ortho to the —C(O)NR³R⁴ group of formula (I).

p is 0-1, and where p is 1, R² is meta to the —C(O)NR³R⁴ group of formula (I) and R² is selected from fluoro or methyl.

p is 1, R² is meta to the —C(O)NR³R⁴ group of formula (I) and R² is selected from fluoro or methyl.

R³ and R⁴ are independently selected from hydrogen, C₁₋₆alkyl, carbocyclyl or heterocyclyl; wherein R³ and R⁴ may be independently optionally substituted on carbon by one or more R⁵; and wherein if said heterocyclyl contains an —NH— moiety that nitrogen may be optionally substituted by a group selected from R⁶; wherein

R⁵ is selected from hydroxy, N,N—(C₁₋₆alkyl)₂amino and heterocyclyl;

R⁶ is selected from C₁₋₆alkyl and C₁₋₆alkoxycarbonyl; wherein R⁶ may be independently optionally substituted on carbon by one or more R¹³;

R¹³ is methoxy.

R³ and R⁴ are independently selected from hydrogen, C₁₋₆alkyl, carbocyclyl or heterocyclyl; wherein R³ and R⁴ may be independently optionally substituted on carbon by one or more R⁵; and wherein if said heterocyclyl contains an —NH— moiety that nitrogen may be optionally substituted by a group selected from R⁶; wherein R⁵ is hydroxy; and R⁶ is C₁₋₆alkyl.

R³ and R⁴ are independently selected from hydrogen, C₁₋₆alkyl or carbocyclyl; wherein R³ and R⁴ may be independently optionally substituted on carbon by one or more R⁵; wherein R⁵ is hydroxy.

R³ and R⁴ are independently selected from hydrogen, methyl, ethyl, isopropyl, cyclopropyl, tetrahydropyranyl, 1,1-dioxidotetrahydrothienyl or piperidinyl; wherein R³ and R⁴ may be independently optionally substituted on carbon by one or more R⁵; and wherein said piperidinyl may be optionally substituted on nitrogen by a group selected from R⁶; wherein

R⁵ is selected from hydroxy, dimethylamino, morpholino, thiomorpholino, pyrrolidinyl and piperidinyl;

R⁶ is selected from methyl, ethyl and t-butoxycarbonyl; wherein R⁶ may be independently optionally substituted on carbon by one or more R¹³;

R¹³ is methoxy.

R³ and R⁴ are independently selected from hydrogen, methyl, ethyl, cyclopropyl, tetrahydrofuranyl or piperidinyl; wherein R³ and R⁴ may be independently optionally substituted on carbon by one or more R⁵; and wherein said piperidinyl may be optionally substituted on nitrogen by a group selected from R⁶; wherein R⁵ is hydroxy; and R⁶ is methyl.

R³ and R⁴ are independently selected from hydrogen, methyl, ethyl or cyclopropyl; wherein R³ and R⁴ may be independently optionally substituted on carbon by one or more R⁵; wherein R⁵ is hydroxy.

R³ and R⁴ are independently selected from hydrogen, methyl, cyclopropyl, 2-hydroxyethyl, 1-methylpiperidin-4-yl, piperidin-3-yl, tetrahydropyran-4-yl, 1,1-dioxidotetrahydrothien-3-yl, 2-dimethylaminoethyl, 1-methyl-2-dimethylaminoethyl, piperidin-1-ylethyl, 2-morpholinoethyl, 1-(2-methoxyethyl)piperidin-4-yl, 2-thiomorpholinoethyl, 2-pyrrolidin-1-ylethyl and 1-(τ-butoxycarbonyl)piperidin-3-yl.

R³ and R⁴ are independently selected from hydrogen, methyl, 2-hydroxyethyl, cyclopropyl, tetrahydrofuran-4-yl or 1-methylpiperidin-4-yl.

R³ and R⁴ are independently selected from hydrogen, methyl, 2-hydroxyethyl or cyclopropyl.

R¹⁹ is selected from ethyl, isopropyl, cyclopropylmethyl, 1-cyclopropylethyl or cyclobutyl.

R¹⁹ is selected from ethyl.

R¹⁹ is selected from isopropyl.

R¹⁹ is selected from cyclopropylmethyl.

R¹⁹ is selected from 1-cyclopropylethyl.

R¹⁹ is selected from cyclobutyl.

R²⁰ is methyl, ethyl, isopropyl, difluoromethyl, trifluoromethyl, methoxymethyl or cyclopropyl.

R²⁰ is methyl.

R²⁰ is ethyl.

R²⁰ is isopropyl.

R²⁰ is difluoromethyl.

R²⁰ is trifluoromethyl.

R²⁰ is methoxymethyl.

R²⁰ is cyclopropyl.

R⁶ and R¹² are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl and N,N—(C₁₋₆alkyl)carbamoyl; wherein R⁶ and R¹² may be independently optionally substituted on carbon by one or more R¹³.

Therefore in a further aspect of the invention there is provided a compound of formula (I) (as depicted above) wherein:

R¹ is hydrogen or fluoro;

R² is halo, cyano or C₁₋₆alkyl;

p is 0 or 1;

R³ and R⁴ are independently selected from hydrogen, C₁₋₆alkyl, carbocyclyl or heterocyclyl; wherein R³ and R⁴ may be independently optionally substituted on carbon by one or more R⁵; and wherein if said heterocyclyl contains an —NH— moiety that nitrogen may be optionally substituted by a group selected from R⁶;

R⁵ is selected from hydroxy, N,N—(C₁₋₆alkyl)₂amino and heterocyclyl;

R⁶ is selected from C₁₋₆alkyl and C₁₋₆alkoxycarbonyl; wherein R⁶ may be independently optionally substituted on carbon by one or more R¹³;

R¹³ is methoxy;

R¹⁹ is selected from ethyl, isopropyl, cyclopropylmethyl, 1-cyclopropylethyl or cyclobutyl;

R²⁰ is methyl, ethyl, isopropyl, difluoromethyl, trifluoromethyl, methoxymethyl or cyclopropyl;

or a pharmaceutically acceptable salt or an in vivo hydrolysable ester thereof.

Therefore in a further aspect of the invention there is provided a compound of formula (I) (as depicted above) wherein:

R¹ is hydrogen or fluoro;

R² is halo, cyano or C₁₋₆alkyl;

p is 0 or 1;

R³ and R⁴ are independently selected from hydrogen, C₁₋₆alkyl, carbocyclyl or heterocyclyl; wherein R³ and R⁴ may be independently optionally substituted on carbon by one or more R⁵; and wherein if said heterocyclyl contains an —NH— moiety that nitrogen may be optionally substituted by a group selected from R⁶; wherein R⁵ is hydroxy; and R⁶ is C₁₋₆alkyl;

R¹⁹ is selected from ethyl, isopropyl, cyclopropylmethyl, 1-cyclopropylethyl or cyclobutyl;

R²⁰ is methyl;

or a pharmaceutically acceptable salt or an in vivo hydrolysable ester thereof.

Therefore in a further aspect of the invention there is provided a compound of formula (I) (as depicted above) wherein:

R¹ is hydrogen or fluoro;

R² is halo or C₁₋₆alkyl;

p is 0 or 1;

R³ and R⁴ are independently selected from hydrogen, C₁₋₆alkyl or carbocyclyl; wherein R³ and R⁴ may be independently optionally substituted on carbon by one or more R⁵; wherein R⁵ is hydroxy;

R¹⁹ is selected from isopropyl; and

R²⁰ is methyl;

or a pharmaceutically acceptable salt or an in vivo hydrolysable ester thereof.

Therefore in a further aspect of the invention there is provided a compound of formula (I) (as depicted above) wherein:

R¹ is hydrogen or fluoro;

R² is fluoro, chloro, cyano or methyl;

p is 0 or 1;

R³ and R⁴ are independently selected from hydrogen, methyl, cyclopropyl, 2-hydroxyethyl, 1-methylpiperidin-4-yl, piperidin-3-yl, tetrahydropyran-4-yl, 1,1-dioxidotetrahydrothien-3-yl, 2-dimethylaminoethyl, 1-methyl-2-dimethylaminoethyl, piperidin-1-ylethyl, 2-morpholinoethyl, 1-(2-methoxyethyl)piperidin-4-yl, 2-thiomorpholinoethyl, 2-pyrrolidin-1-ylethyl and 1-(τ-butoxycarbonyl)piperidin-3-yl;

R¹⁹ is selected front ethyl, isopropyl, cyclopropylmethyl, 1-cyclopropylethyl or cyclobutyl;

R²⁰ is methyl, ethyl, isopropyl, difluoromethyl, trifluoromethyl, methoxymethyl or cyclopropyl;

or a pharmaceutically acceptable salt or an in vivo hydrolysable ester thereof.

Therefore in a further aspect of the invention there is provided a compound of formula (I) (as depicted above) wherein:

R¹ is hydrogen or fluoro;

R² is fluoro, chloro, cyano or methyl;

p is 0 or 1;

R³ and R⁴ are independently selected from hydrogen, methyl, 2-hydroxyethyl, cyclopropyl, tetrahydrofuran-4-yl or 1-methylpiperidin-4-yl;

R¹⁹ is selected from ethyl, isopropyl, cyclopropylmethyl, 1-cyclopropylethyl or cyclobutyl;

R²⁰ is methyl;

or a pharmaceutically acceptable salt or an in vivo hydrolysable ester thereof.

Therefore in a further aspect of the invention there is provided a compound of formula (I) (as depicted above) wherein

R¹ is hydrogen or fluoro;

R² is fluoro, chloro or methyl;

p is 0 or 1;

R³ and R⁴ are independently selected from hydrogen, methyl, 2-hydroxyethyl or cyclopropyl;

R¹⁹ is selected from isopropyl; and

R²⁰ is methyl;

or a pharmaceutically acceptable salt or an in vivo hydrolysable ester thereof.

In another aspect of the invention, preferred compounds of the invention are any one of the Examples or a pharmaceutically acceptable salt or an in vivo hydrolysable ester thereof.

In another aspect of the invention there is provided a compound of formula (I) selected from Examples 9, 13, 14, 34, 51, 79, 80, 81, 90 and 94 or a pharmaceutically acceptable salt or an in vivo hydrolysable ester thereof.

Preferred aspects of the invention are those which relate to the compound of formula (I) or a pharmaceutically acceptable salt thereof.

Another aspect of the present invention provides a process for preparing a compound of formula (I) or a pharmaceutically acceptable salt or an in vivo hydrolysable ester thereof which process (wherein variable groups are, unless otherwise specified, as defined in formula (I)) comprises of:

Process a) reaction of a pyrimidine of formula (II):

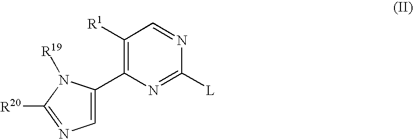

wherein L is a displaceable group; with an aniline of formula (III):

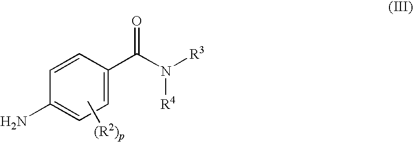

or
Process b) reacting a compound of formula (IV):

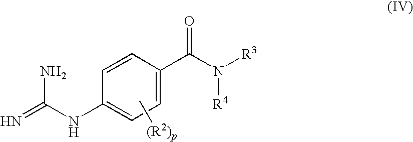

with a compound of formula (V):

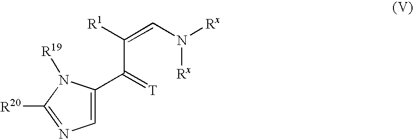

wherein T is O or S; R^{x} may be the same or different and is selected from C₁₋₆alkyl; or
Process c) reacting an acid of formula (VI):

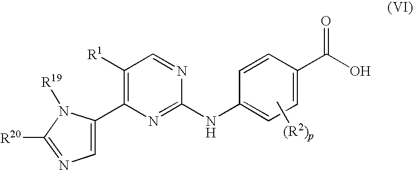

or an activated derivative thereof; with an amine of formula (VII):

HNR³R⁴ (VII)

or
Process d) for compounds of formula (I); reacting a pyrimidine of formula (VIII)

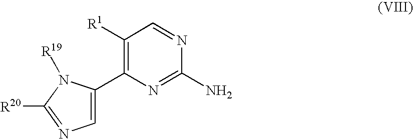

with a compound of formula (IX):

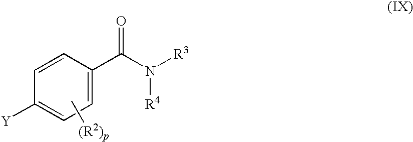

where Y is a displaceable group;
and thereafter if necessary:
i) converting a compound of the formula (I) into another compound of the formula (I);
ii) removing any protecting groups;
iii) forming a pharmaceutically acceptable salt or in vivo hydrolysable ester.

L is a displaceable group, suitable values for L are for example, a halogeno or sulphonyloxy group, for example a chloro, bromo, methanesulphonyloxy or toluene-4-sulphonyloxy group.

Y is a displaceable group, suitable values for Y are for example, a halogeno or sulphonyloxy group, for example a bromo, iodo or trifluoromethanesulphonyloxy group. Preferably Y is iodo.

Specific reaction conditions for the above reactions are as follows.

Process a) Pyrimidines of formula (II) and anilines of formula (III) may be reacted together:
i) in the presence of a suitable solvent for example a ketone such as acetone or an alcohol such as ethanol or butanol or an aromatic hydrocarbon such as toluene or N-methyl pyrrolidine, optionally in the presence of a suitable acid for example an inorganic acid such as hydrochloric acid or sulphuric acid, or an organic acid such as acetic acid or formic acid (or a suitable Lewis acid) and at a temperature in the range of 0° C. to reflux, preferably reflux; or
ii) under standard Buchwald conditions (for example see J. Am. Chem. Soc., 118, 7215; J. Am. Chem. Soc., 119, 8451; J. Org. Chem., 62, 1568 and 6066) for example in the presence of palladium acetate, in a suitable solvent for example an aromatic solvent such as toluene, benzene or xylene, with a suitable base for example an inorganic base such as caesium carbonate or an organic base such as potassium-t-butoxide, in the presence of a suitable ligand such as 2,2′-bis(diphenylphosphino)-1,1′-binaphthyl and at a temperature in the range of 25 to 80° C.

Pyrimidines of the formula (II) where L is chloro may be prepared according to Scheme 1:

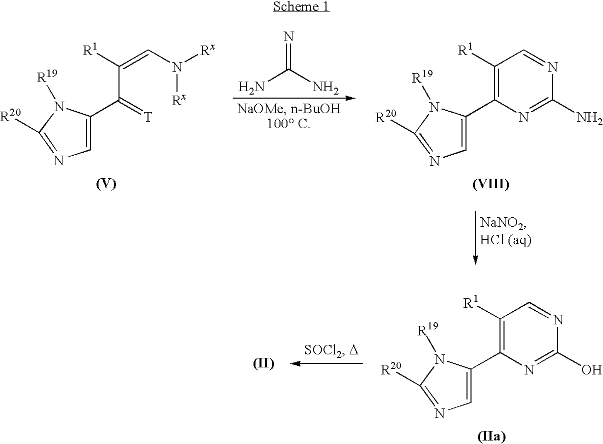

Anilines of formula (III) are commercially available compounds, or they are known in the literature, or they are prepared by standard processes known in the art.

Process b) Compounds of formula (IV) and compounds of formula (V) are reacted together in a suitable solvent such as N-methylpyrrolidinone or butanol at a temperature in the range of 100-200° C., preferably in the range of 150-170° C. The reaction is preferably conducted in the presence of a suitable base such as, for example, sodium hydride, sodium methoxide or potassium carbonate.

Compounds of formula (V) may be prepared according to Scheme 2:

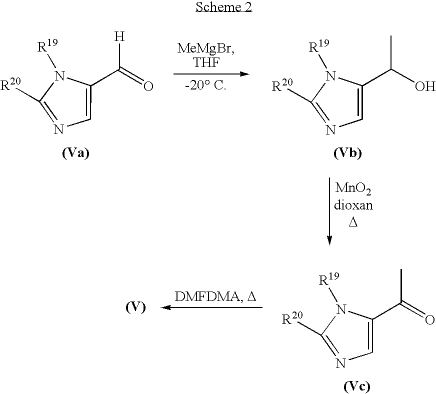

Compounds of formula (IV) and (Va) are commercially available compounds, or they are known in the literature, or they are prepared by standard processes known in the art.

Process c) Acids and amines may be coupled together in the presence of a suitable coupling reagent. Standard peptide coupling reagents known in the art can be employed as suitable coupling reagents, or for example carbonyldiimidazole and dicyclohexyl-carbodiimide, optionally in the presence of a catalyst such as dimethylaminopyridine or 4-pyrrolidinopyridine, optionally in the presence of a base for Example triethylamine, pyridine, or 2,6-di-alkyl-pyridines such as 2,6-lutidine or 2,6-di-tert-butylpyridine. Suitable solvents include dimethylacetamide, dichloromethane, benzene, tetrahydrofuran and dimethylformamide. The coupling reaction may conveniently be performed at a temperature in the range of −40 to 40° C.

Suitable activated acid derivatives include acid halides, for example acid chlorides, and active esters, for example pentafluorophenyl esters. The reaction of these types of compounds with amines is well known in the art, for example they may be reacted in the presence of a base, such as those described above, and in a suitable solvent, such as those described above. The reaction may conveniently be performed at a temperature in the range of −40 to 40° C.

Compounds of formula (VI) may be prepared by adapting Process a), b) or c).

Amines of formula (VII) are commercially available compounds, or they are known in the literature, or they are prepared by standard processes known in the art.

Process d) Compounds of formula (VIII) and amines of formula (IX) may be reacted together under standard Buchwald conditions as described in Process a.

The synthesis of compounds of formula (VIII) is described in Scheme 1.

Compounds of formula (IX) are commercially available compounds, or they are known in the literature, or they are prepared by standard processes known in the art.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a τ-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a 1-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

As stated hereinbefore the compounds defined in the present invention possesses anti-cell-proliferation activity such as anti-cancer activity which is believed to arise from the CDK inhibitory activity of the compound. These properties may be assessed, for example, using the procedure set out below:—

### Assay

The following abbreviations have been used:—

HEPES is N-[2-Hydroxyethyl]piperazine-N′-[2-ethanesulfonic acid]

### DTT is Dithiothreitol

PMSF is Phenylmethylsulphonyl fluoride

The compounds were tested in an in vitro kinase assay in 96 well format using Scintillation Proximity Assay (SPA—obtained from Amersham) for measuring incorporation of [γ-33-P]-Adenosine Triphosphate into a test substrate (GST-Retinoblastoma protein; GST-Rb). In each well was placed the compound to be tested (diluted in DMSO and water to correct concentrations) and in control wells either roscovitine as an inhibitor control or DMSO as a positive control.

Approximately 0.2 μl of CDK2/Cyclin E partially-purified enzyme (amount dependent on enzyme activity) diluted in 25 μl incubation buffer was added to each well then 20 μl of GST-Rb/ATP/ATP33 mixture (containing 0.5 μg GST-Rb and 0.2 μM ATP and 0.14 μCi [γ-33-P]-Adenosine Triphosphate in incubation buffer), and the resulting mixture shaken gently, then incubated at room temperature for 60 minutes.

To each well was then added 150 μL stop solution containing (0.8 mg/well of Protein A-PVT SPA bead (Amersham)), 20 μM/well of Anti-Glutathione Transferase, Rabbit IgG (obtained from Molecular Probes), 61 mM EDTA and 50 mM HEPES pH 7.5 containing 0.05% sodium azide.

The plates were sealed with Topseal-S plate sealers, left for two hours then spun at 2500 rpm, 1124 xg., for 5 minutes. The plates were read on a Topcount for 30 seconds per well.

The incubation buffer used to dilute the enzyme and substrate mixes contained 50 mM HEPES pH7.5, 10 mM MnCl₂, 1 mM DTT, 100 μM Sodium vanadate, 100 μM NaF, 10 mM Sodium Glycerophosphate, BSA (1 mg/ml final).

### Test Substrate

In this assay only part of the retinoblastoma protein (Science 1987 Mar. 13; 235(4794):1394-1399; Lee W. H., Bookstein R., Hong F., Young L. J., Shew J. Y., Lee E. Y.) was used, fused to a GST tag. PCR of retinoblastoma gene encoding amino acids 379-928 (obtained from retinoblastoma plasmid ATCC pLRbRNL) was performed, and the sequence cloned into pGEx 2T fusion vector (Smith D. B. and Johnson, K. S. Gene 67, 31 (1988); which contained a tac promoter for inducible expression, internal lac I^{q} gene for use in any E. Coli host, and a coding region for thrombin cleavage—obtained from Pharmacia Biotech) which was used to amplify amino acids 792-928. This sequence was again cloned into pGEx 2T.

The retinoblastoma 792-928 sequence so obtained was expressed in E. Coli (BL21 (DE3) pLysS cells) using standard inducible expression techniques, and purified as follows.

E. coli paste was resuspended in 10 ml/g of NETN buffer (50 mM Tris pH 7.5, 120 mM NaCl, 1 mM EDTA, 0.5% v/v NP-40, 1 mM PMSF, 1 μg/ml leupeptin, 1 ug/ml aprotinin and 1 ug/ml pepstatin) and sonicated for 2×45 seconds per 100 ml homogenate. After centrifugation, the supernatant was loaded onto a 10 ml glutathione Sepharose column (Pharmacia Biotech, Herts, UK), and washed with NETN buffer. After washing with kinase buffer (50 mM HEPES pH 7.5, 10 mM MgCl₂, 1 mM DTT, 1 mM PMSF, 1 ug/ml leupeptin, 1 ug/ml aprotinin and 1 ug/ml pepstatin) the protein was eluted with 50 mM reduced glutathione in kinase buffer. Fractions containing GST-Rb (792-927) were pooled and dialysed overnight against kinase buffer. The final product was analysed by Sodium Dodeca Sulfate (SDS) PAGE (Polyacrylamide gel) using 8-16% Tris-Glycine gels (Novex, San Diego, USA).

### CDK2 and Cyclin E

The open reading frames of CDK2 and Cyclin E were isolated by reverse transcriptase-PCR using HeLa cell and activated T cell mRNA as a template and cloned into the insect expression vector pVL1393 (obtained from Invitrogen 1995 catalogue number: V1392-20). CDK2 and cyclin E were then dually expressed [using a standard virus Baculogold co-infection technique] in the insect SF21 cell system (Spodoptera Frugiperda cells derived from ovarian tissue of the Fall Army Worm—commercially available).

Example production of Cyclin E/CDK2

The following Example provides details of the production of Cyclin E/CDK2 in SF21 cells (in TC100+10% FBS(TCS)+0.2% Pluronic) having dual infection MOI 3 for each virus of Cyclin E & CDK2.

SF21 cells grown in a roller bottle culture to 2.33×10⁶ cells/ml were used to inoculate 10×500 ml roller bottles at 0.2×10E6 cells/ml. The roller bottles were incubated on a roller rig at 28° C.

After 3 days (72 hrs.) the cells were counted, and the average from 2 bottles found to be 1.86×10E6 cells/ml. (99% viable). The cultures were then infected with the dual viruses at an MOI 3 for each virus.

The viruses were mixed together before addition to the cultures, and the cultures returned to the roller rig 28° C.

After 2 days (48 hrs.) post infection the 5 Litres of culture was harvested. The total cell count at harvest was 1.58×10E6 cells/ml. (99% viable). The cells were spun out at 2500 rpm, 30 mins., 4° C. in Heraeus Omnifuge 2.0 RS in 250 ml. lots. The supernatant was discarded.

### Partial Co-Purification of CDK2 and Cyclin E

Sf21 cells were resuspended in lysis buffer (50 mM Tris pH 8.2, 10 mM MgCl₂, 1 mM DTT, 10 mM glycerophosphate, 0.1 mM sodium orthovanadate, 0.1 mM NaF, 1 mM PMSF, 1 ug/ml leupeptin and 1 ug/ml aprotinin) and homogenised for 2 minutes in a 10 ml Dounce homgeniser. After centrifugation, the supernatant was loaded onto a Poros HQ/M 1.4/100 anion exchange column (PE Biosystems, Hertford, UK). Cdk2 and Cyclin E were coeluted at the beginning of a 0-1M NaCl gradient (run in lysis buffer minus protease inhibitors) over 20 column volumes. Co-elution was checked by western blot using both anti-Cdk2 and anti-Cyclin E antibodies (Santa Cruz Biotechnology, California, US).

By analogy, assays designed to assess inhibition of CDK1 and CDK4 may be constructed. CDK2 (EMBL Accession No. X62071) may be used together with Cyclin A or Cyclin E (see EMBL Accession No. M73812), and further details for such assays are contained in PCT International Publication No. WO99/21845, the relevant Biochemical & Biological Evaluation sections of which are hereby incorporated by reference.

Although the pharmacological properties of the compounds of the formula (I) vary with structural change, in general activity possessed by compounds of the formula (I) may be demonstrated at IC₅₀ concentrations or doses in the range 250 μM to 1 nM.

When tested in the above in-vitro assay the CDK2 inhibitory activity of Example 28 was measured as IC₅₀=0.003 μM.

The in vivo activity of the compounds of the present invention may be assessed by standard techniques, for example by measuring inhibition of cell growth and assessing cytotoxicity.

Inhibition of cell growth may be measured by staining cells with Sulforhodamine B (SRB), a fluorescent dye that stains proteins and therefore gives an estimation of amount of protein (i.e. cells) in a well (see Boyd, M. R. (1989) Status of the NCI preclinical antitumour drug discovery screen. Prin. Prac Oncol 10:1-12). Thus, the following details are provided of measuring inhibition of cell growth:—

Cells may be plated in appropriate medium in a volume of 100 ml in 96 well plates; the media can be Dulbecco's Modified Eagle media for MCF-7, SK-UT-1B and SK-UT-1. The cells can be allowed to attach overnight, then inhibitor compounds may be added at various concentrations in a maximum concentration of 1% DMSO (v/v). A control plate may be assayed to give a value for cells before dosing. Cells may be incubated at 37° C., (5% CO₂) for three days.

At the end of three days TCA may be added to the plates to a final concentration of 16% (v/v). Plates may be incubated at 4° C. for 1 hour, the supernatant removed and the plates washed in tap water. After drying, 100 ml SRB dye (0.4% SRB in 1% acetic acid) may be added for 30 minutes at 37° C. Excess SRB may be removed and the plates washed in 1% acetic acid. The SRB bound to protein may be solubilised in 10 mM Tris pH7.5 and shaken for 30 minutes at room temperature. The ODs may be read at 540 nm, and the concentration of inhibitor causing 50% inhibition of growth determined from a semi-log plot of inhibitor concentration versus absorbance. The concentration of compound that reduced the optical density to below that obtained when the cells were plated at the start of the experiment will give the value for toxicity.

Typical IC₅₀ values for compounds of the invention when tested in the SRB assay would be in the range 1 mM to 1 nM.

The level of oral exposure of a compound can be measured by the following assay. This assay gives a semi-quantitative measure of the concentration of the compound achieved in the blood at a number of time points. Data available include Cmax (highest concentration achieved), and the AUC (area under the plasma concentration/time curve) for the compound. This gives a high throughput measure of likelihood of obtaining blood levels for each compound following oral dosing and as the data are normalised for dose, it allows direct comparison of each compound.

High Throughput Blood Level Assay—Rat

A cocktail of 6 compounds is formulated in propylene glycol using a combination of vortex mixing, sonication and high speed shear mixing. This formulation consists of 5 test compounds (1 mg/ml) and a standard (0.5 mg/ml). The resulting formulation is a solution or a stable (≧several hours) suspension.

The formulation is dosed (2 ml/kg) to two male rats (170-250 gm) which have been fasted for ≦16 hours then pre-dosed with water (˜10 ml/kg). The dose for the test compounds is 2 mg/kg and for the standard it is 1 mg/kg.

Serial blood samples are taken from rats at 0.5, 1, 2 and 4 hours post dose via the tail vein and a terminal sample is taken at 6-hour post dose.

The blood samples are centrifuged and plasma removed for analysis. The two plasma samples for a given time point are combined prior to analysis.

A single set of 6 calibration standards containing all 6 compounds covering the concentration range (0.3 ng/ml to 3 μg/ml) are prepared by spiking blank plasma. The samples and standards are extracted by precipitation with 2 volumes of acetonitrile followed by centrifugation. The resulting supernatant is then diluted with water (10 fold).

Samples are analysed by LC/MS-MS and the concentration obtained is used to determine the Cmax μg/ml (maximum compound level detected), AUC0-6 hr μg/hr/ml (area under the curve) and tmax hr (time that the maximum compound levels have been measured) for a given compound to give an indication of exposure

### High Throughput Blood Level Assay—Mice

The above assay may be run using mice in place of rats, but with the following variations.

For a profile in mice 10 male mice are dosed at the same level as to rats but they are not fasted or predosed with water. Furthermore, samples from mice are all terminal, with 2 mice per time point.

When tested in the above assay Example 25, in the AP rat (see below), normalised to a 1 mg/kg dose, Cmax was 0.3995 μM, and AUC was 0.8295 μM.h. In the AP mouse (see below), again normalised to 1 mg/kg, Cmax=0.68 μM, and AUC was 1.50 μM.h.

Note the rats used in the above experiment were Alderley Park (AP) rats. The AP rat is a Wistar derived animal imported into ICI from the Wistar unit via Porton Down in the 1940's. The stock was rederived by fostering onto Sprague Dawleys and has remained a closed colony since 1959. The nomenclature for these animals is Alpk..APfSD.

The mice used in the above experiment were AP mice. The AP mouse was originally obtained from a commercial breeder Schoefields in 1956. The stock was rederived by fostering onto a CD-1 Charles Rivers (commercial supplier) outbred mouse and has remained a closed colony ever since. The nomenclature for these animals is Alpk..APfCD-1.

According to a further aspect of the invention there is provided a pharmaceutical composition which comprises a pyrimidine derivative of the formula (I), or a pharmaceutically acceptable salt or in vivo hydrolysable ester thereof, as defined hereinbefore in association with a pharmaceutically-acceptable diluent or carrier.

The composition may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository.

In general the above compositions may be prepared in a conventional manner using conventional excipients.

The compound of formula (I) will normally be administered to a warm-blooded animal at a unit dose within the range 5-5000 mg per square meter body area of the animal, i.e. approximately 0.1-100 mg/kg, and this normally provides a therapeutically-effective dose. A unit dose form such as a tablet or capsule will usually contain, for example 1-250 mg of active ingredient. Preferably a daily dose in the range of 1-50 mg/kg is employed. However the daily dose will necessarily be varied depending upon the host treated, the particular route of administration, and the severity of the illness being treated. Accordingly the optimum dosage may be determined by the practitioner who is treating any particular patient.

According to a further aspect of the present invention there is provided a compound of the formula (I), or a pharmaceutically acceptable salt or in vivo hydrolysable ester thereof, as defined hereinbefore for use in a method of treatment of the human or animal body by therapy.

We have found that the compounds defined in the present invention, or a pharmaceutically acceptable salt or in vivo hydrolysable ester thereof, are effective cell cycle inhibitors (anti-cell proliferation agents), which property is believed to arise from their CDK inhibitory properties. Accordingly the compounds of the present invention are expected to be useful in the treatment of diseases or medical conditions mediated alone or in part by CDK enzymes, i.e. the compounds may be used to produce a CDK inhibitory effect in a warm-blooded animal in need of such treatment. Thus the compounds of the present invention provide a method for treating the proliferation of malignant cells characterised by inhibition of CDK enzymes, i.e. the compounds may be used to produce an anti-proliferative effect mediated alone or in part by the inhibition of CDKs. Such a compound of the invention is expected to possess a wide range of anti-cancer properties as CDKs have been implicated in many common human cancers such as leukaemia and breast, lung, colon, rectal, stomach, prostate, bladder, pancreas and ovarian cancer. Thus it is expected that a compound of the invention will possess anti-cancer activity against these cancers. It is in addition expected that a compound of the present invention will possess activity against a range of leukaemias, lymphoid malignancies and solid tumours such as carcinomas and sarcomas in tissues such as the liver, kidney, prostate and pancreas. In particular such compounds of the invention are expected to slow advantageously the growth of primary and recurrent solid tumours of, for example, the colon, breast, prostate, lungs and skin. More particularly such compounds of the invention, or a pharmaceutically acceptable salt or in vivo hydrolysable ester thereof, are expected to inhibit the growth of those primary and recurrent solid tumours which are associated with CDKs, especially those tumours which are significantly dependent on CDKs for their growth and spread, including for example, certain tumours of the colon, breast, prostate, lung, vulva and skin.

It is further expected that a compound of the present invention will possess activity against other cell-proliferation diseases in a wide range of other disease states including leukaemias, fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation.

Thus according to this aspect of the invention there is provided a compound of the formula (I), or a pharmaceutically acceptable salt or in vivo hydrolysable ester thereof, as defined hereinbefore for use as a medicament; and the use of a compound of the formula (I), or a pharmaceutically acceptable salt or in vivo hydrolysable ester thereof, as defined hereinbefore in the manufacture of a medicament for use in the production of a cell cycle inhibitory (anti-cell-proliferation) effect in a warm-blooded animal such as man. Particularly, an inhibitory effect is produced by preventing entry into, or progression through, the S phase by inhibition of CDK2 and CDK4, especially CDK2, and M phase by inhibition of CDK1.

According to a further feature of the invention, there is provided a compound of the formula (I), or a pharmaceutically acceptable salt or in vivo hydrolysable ester thereof, as defined herein before in the manufacture of a medicament for use in the treatment of cancers (solid tumours and leukaemias), fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation, particularly in the treatment of cancers.

According to a further feature of this aspect of the invention there is provided a method for producing a cell cycle inhibitory (anti-cell-proliferation) effect in a warm-blooded animal, such as man, in need of such treatment which comprises administering to said animal an effective amount of a compound as defined immediately above. Particularly, an inhibitory effect is produced by preventing entry into, or progression through, the S phase by inhibition of CDK2 and CDK4, especially CDK2, and M phase by inhibition of CDK1.

According to a further feature of this aspect of the invention there is provided a method for producing a cell cycle inhibitory (anti-cell-proliferation) effect in a warm-blooded animal, such as man, in need of such treatment which comprises administering to said animal an effective amount of a compound of formula (I) or a pharmaceutically acceptable salt or in vivo hydrolysable ester thereof as defined herein before. Particularly, an inhibitory effect is produced by preventing entry into, or progression through, the S phase by inhibition of CDK2 and CDK4, especially CDK2, and M phase by inhibition of CDK1.

According to an additional feature of this aspect of the invention there is provided a method of treating cancers (solid tumours and leukaemias), fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation, in a warm-blooded animal, such as man, in need of such treatment which comprises administering to said animal an effective amount of a compound of formula (I) or a pharmaceutically acceptable salt or in vivo hydrolysable ester thereof as defined herein before.

Particularly there is provided a method of treating cancer in a warm-blooded animal, such as man, in need of such treatment which comprises administering to said animal an effective amount of a compound of formula (I) or a pharmaceutically acceptable salt or in vivo hydrolysable ester thereof as defined herein before.

In a further aspect of the invention there is provided a pharmaceutical composition which comprises a compound of the formula (I), or a pharmaceutically acceptable salt or in vivo hydrolysable ester thereof, as defined herein before in association with a pharmaceutically-acceptable diluent or carrier for use in the production of a cell cycle inhibitory (anti-cell-proliferation) effect in a warm-blooded animal such as man.

In a further aspect of the invention there is provided a pharmaceutical composition which comprises a compound of the formula (I), or a pharmaceutically acceptable salt or in vivo hydrolysable ester thereof, as defined herein before in association with a pharmaceutically-acceptable diluent or carrier for use in the treatment of cancers (solid tumours and leukaemias), fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation, in a warm-blooded animal such as man.

In a further aspect of the invention there is provided a pharmaceutical composition which comprises a compound of the formula (I), or a pharmaceutically acceptable salt or in vivo hydrolysable ester thereof, as defined herein before in association with a pharmaceutically-acceptable diluent or carrier for use in the treatment of cancer in a warm-blooded animal such as man.

Thus according to this aspect of the invention there is provided a compound of the formula (I), or a pharmaceutically acceptable salt or in vivo hydrolysable ester thereof, as defined hereinbefore for use as a medicament.

In a further aspect of the invention there is provided the use of a compound of the formula (I), or a pharmaceutically acceptable salt or in vivo hydrolysable ester thereof, as defined hereinbefore in the manufacture of a medicament for use in the production of a cell cycle inhibitory effect.

In a further aspect of the invention there is provided the use of a compound of the formula (I), or a pharmaceutically acceptable salt or in vivo hydrolysable ester thereof, as defined hereinbefore in the manufacture of a medicament for use in the production of an anti-cell-proliferation effect.

In a further aspect of the invention there is provided the use of a compound of the formula (I), or a pharmaceutically acceptable salt or in vivo hydrolysable ester thereof, as defined hereinbefore in the manufacture of a medicament for use in the production of a CDK2 inhibitory effect.

In a further aspect of the invention there is provided the use of a compound of the formula (I), or a pharmaceutically acceptable salt or in vivo hydrolysable ester thereof, as defined hereinbefore in the manufacture of a medicament for use in the treatment of cancer.

In a further aspect of the invention there is provided the use of a compound of the formula (I), or a pharmaceutically acceptable salt or in vivo hydrolysable ester thereof, as defined hereinbefore in the manufacture of a medicament for use in the treatment of leukaemia or lymphoid malignancies or cancer of the breast, lung, colon, rectum, stomach, liver, kidney, prostate, bladder, pancreas, vulva, skin or ovary.

According to a further feature of the invention, there is provided the use of a compound of the formula (I), or a pharmaceutically acceptable salt or in vivo hydrolysable ester thereof, as defined herein before in the manufacture of a medicament for use in the treatment of cancer, fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation.

In a further aspect of the invention there is provided a method of producing a cell cycle inhibitory effect, in a warm-blooded animal in need of such treatment, which comprises administering to said animal an effective amount of a compound of formula (I) or a pharmaceutically acceptable salt or in vivo hydrolysable ester thereof, as defined herein before.

In a further aspect of the invention there is provided a method of producing an anti-cell-proliferation effect, in a warm-blooded animal in need of such treatment, which comprises administering to said animal an effective amount of a compound of formula (I) or a pharmaceutically acceptable salt or in vivo hydrolysable ester thereof, as defined herein before.

In a further aspect of the invention there is provided a method of producing a CDK2 inhibitory effect, in a warm-blooded animal in need of such treatment, which comprises administering to said animal an effective amount of a compound of formula (I) or a pharmaceutically acceptable salt or in vivo hydrolysable ester thereof, as defined herein before.

In a further aspect of the invention there is provided a method of treating cancer, in a warm-blooded animal in need of such treatment, which comprises administering to said animal an effective amount of a compound of formula (I) or a pharmaceutically acceptable salt or in vivo hydrolysable ester thereof, as defined herein before.

In a further aspect of the invention there is provided a method of treating leukaemia or lymphoid malignancies or cancer of the breast, lung, colon, rectum, stomach, liver, kidney, prostate, bladder, pancreas, vulva, skin or ovary, in a warm-blooded animal in need of such treatment, which comprises administering to said animal an effective amount of a compound of formula (I) or a pharmaceutically acceptable salt or in vivo hydrolysable ester thereof, as defined herein before.

In a further aspect of the invention there is provided a method of treating cancer, fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation, in a warm-blooded animal in need of such treatment, which comprises administering to said animal an effective amount of a compound of formula (I) or a pharmaceutically acceptable salt or in vivo hydrolysable ester thereof, as defined herein before.

In a further aspect of the invention there is provided a pharmaceutical composition which comprises a compound of the formula (I), or a pharmaceutically acceptable salt or in vivo hydrolysable ester thereof, as defined herein before and a pharmaceutically-acceptable diluent or carrier.

In a further aspect of the invention there is provided a pharmaceutical composition which comprises a compound of the formula (I), or a pharmaceutically acceptable salt or in vivo hydrolysable ester thereof, as defined herein before and a pharmaceutically-acceptable diluent or carrier for use as a medicament.

In a further aspect of the invention there is provided a pharmaceutical composition which comprises a compound of the formula (I), or a pharmaceutically acceptable salt or in vivo hydrolysable ester thereof, as defined herein before and a pharmaceutically-acceptable diluent or carrier for use in the production of a cell cycle inhibitory effect.

In a further aspect of the invention there is provided a pharmaceutical composition which comprises a compound of the formula (I), or a pharmaceutically acceptable salt or in vivo hydrolysable ester thereof, as defined herein before and a pharmaceutically-acceptable diluent or carrier for use in the production of an anti-cell-proliferation effect.

In a further aspect of the invention there is provided a pharmaceutical composition which comprises a compound of the formula (I), or a pharmaceutically acceptable salt or in vivo hydrolysable ester thereof, as defined herein before and a pharmaceutically-acceptable diluent or carrier for use in the production of a CDK2 inhibitory effect.

In a further aspect of the invention there is provided a pharmaceutical composition which comprises a compound of the formula (I), or a pharmaceutically acceptable salt or in vivo hydrolysable ester thereof, as defined herein before and a pharmaceutically-acceptable diluent or carrier for use in the treatment of cancer.

In a further aspect of the invention there is provided a pharmaceutical composition which comprises a compound of the formula (I), or a pharmaceutically acceptable salt or in vivo hydrolysable ester thereof, as defined herein before and a pharmaceutically-acceptable diluent or carrier for use in the treatment of leukaemia or lymphoid malignancies or cancer of the breast, lung, colon, rectum, stomach, liver, kidney, prostate, bladder, pancreas, vulva, skin or ovary.

In a further aspect of the invention there is provided a pharmaceutical composition which comprises a compound of the formula (I), or a pharmaceutically acceptable salt or in vivo hydrolysable ester thereof, as defined herein before and a pharmaceutically-acceptable diluent or carrier for use in the treatment of cancer, fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation.

In a further aspect of the invention there is provided the use of a compound of the formula (I), or a pharmaceutically acceptable salt or in vivo hydrolysable ester thereof, as defined hereinbefore, in the production of a cell cycle inhibitory effect.

In a further aspect of the invention there is provided the use of a compound of the formula (I), or a pharmaceutically acceptable salt or in vivo hydrolysable ester thereof, as defined hereinbefore, in the production of an anti-cell-proliferation effect.

In a further aspect of the invention there is provided the use of a compound of the formula (I), or a pharmaceutically acceptable salt or in vivo hydrolysable ester thereof, as defined hereinbefore, in the production of a CDK2 inhibitory effect.

In a further aspect of the invention there is provided the use of a compound of the formula (I), or a pharmaceutically acceptable salt or in vivo hydrolysable ester thereof, as defined hereinbefore, in the treatment of cancer.

In a further aspect of the invention there is provided the use of a compound of the formula (I), or a pharmaceutically acceptable salt or in vivo hydrolysable ester thereof, as defined hereinbefore in the treatment of leukaemia or lymphoid malignancies or cancer of the breast, lung, colon, rectum, stomach, liver, kidney, prostate, bladder, pancreas, vulva, skin or ovary.

According to a further feature of the invention, there is provided the use of a compound of the formula (I), or a pharmaceutically acceptable salt or in vivo hydrolysable ester thereof, as defined herein before in the treatment of cancer, fibroproliferative and differentiative disorders, psoriasis, rheumatoid arthritis, Kaposi's sarcoma, haemangioma, acute and chronic nephropathies, atheroma, atherosclerosis, arterial restenosis, autoimmune diseases, acute and chronic inflammation, bone diseases and ocular diseases with retinal vessel proliferation.

Preventing cells from entering DNA synthesis by inhibition of essential S-phase initiating activities such as CDK2 initiation may also be useful in protecting normal cells of the body from toxicity of cycle-specific pharmaceutical agents. Inhibition of CDK2 or 4 will prevent progression into the cell cycle in normal cells which could limit the toxicity of cycle-specific pharmaceutical agents which act in S-phase, G2 or mitosis. Such protection may result in the prevention of hair loss normally associated with these agents.

Therefore in a further aspect of the invention there is provided a compound of formula (I) as defined above or a pharmaceutically acceptable salt or in vivo hydrolysable ester thereof for use as a cell protective agent.

Therefore in a further aspect of the invention there is provided a compound of formula (I) as defined above or a pharmaceutically acceptable salt or in vivo hydrolysable ester thereof for use in preventing hair loss arising from the treatment of malignant conditions with pharmaceutical agents.

Examples of pharmaceutical agents for treating malignant conditions that are known to cause hair loss include alkylating agents such as ifosfamide and cyclophosphamide; antimetabolites such as methotrexate, 5-fluorouracil, gemcitabine and cytarabine; vinca alkaloids and analogues such as vincristine, vinbalstine, vindesine, vinorelbine; taxanes such as paclitaxel and docetaxel; topoisomerase I inhibitors such as irintotecan and topotecan; cytotoxic antibiotics such as doxorubicin, daunorubicin, mitoxantrone, actinomycin-D and mitomycin; and others such as etoposide and tretinoin.

In another aspect of the invention, the compound of formula (I), or a pharmaceutically acceptable salt or in vivo hydrolysable ester thereof, may be administered in association with a one or more of the above pharmaceutical agents. In this instance the compound of formula (I) may be administered by systemic or non systemic means. Particularly the compound of formula (I) my may administered by non-systemic means, for example topical administration.

Therefore in an additional feature of the invention, there is provided a method of preventing hair loss during treatment for one or more malignant conditions with pharmaceutical agents, in a warm-blooded animal, such as man, which comprises administering to said animal an effective amount of a compound of formula (I), or a pharmaceutically acceptable salt or in vivo hydrolysable ester thereof.

In an additional feature of the invention, there is provided a method of preventing hair loss during treatment for one or more malignant conditions with pharmaceutical agents, in a warm-blooded animal, such as man, which comprises administering to said animal an effective amount of a compound of formula (I), or a pharmaceutically acceptable salt or in vivo hydrolysable ester thereof in simultaneous, sequential or separate administration with an effective amount of said pharmaceutical agent.

According to a further aspect of the invention there is provided a pharmaceutical composition for use in preventing hair loss arising from the treatment of malignant conditions with pharmaceutical agents which comprises a compound of formula (I), or a pharmaceutically acceptable salt or in vivo hydrolysable ester thereof, and said pharmaceutical agent, in association with a pharmaceutically acceptable diluent or carrier.

According to a further aspect of the present invention there is provided a kit comprising a compound of formula (I), or a pharmaceutically acceptable salt or in vivo hydrolysable ester thereof, and a pharmaceutical agent for treating malignant conditions that is known to cause hair loss.

According to a further aspect of the present invention there is provided a kit comprising:

a) a compound of formula (I), or a pharmaceutically acceptable salt or in vivo hydrolysable ester thereof, in a first unit dosage form;
b) a pharmaceutical agent for treating malignant conditions that is known to cause hair loss; in a second unit dosage form; and
c) container means for containing said first and second dosage forms.

According to another feature of the invention there is provided the use of a compound of the formula (I), or a pharmaceutically acceptable salt or in vivo hydrolysable ester thereof, in the manufacture of a medicament for the prevention of hair loss during treatment of malignant conditions with pharmaceutical agents.

According to a further aspect of the present invention there is provided a combination treatment for the prevention of hair loss comprising the administration of an effective amount of a compound of the formula (I), or a pharmaceutically acceptable salt or in vivo hydrolysable ester thereof, optionally together with a pharmaceutically acceptable diluent or carrier, with the simultaneous, sequential or separate administration of an effective amount of a pharmaceutical agent for treatment of malignant conditions to a warm-blooded animal, such as man.

As stated above the size of the dose required for the therapeutic or prophylactic treatment of a particular cell-proliferation disease will necessarily be varied depending on the host treated, the route of administration and the severity of the illness being treated. A unit dose in the range, for example, 1-100 mg/kg, preferably 1-50 mg/kg is envisaged.

The CDK inhibitory activity defined hereinbefore may be applied as a sole therapy or may involve, in addition to a compound of the invention, one or more other substances and/or treatments. Such conjoint treatment may be achieved by way of the simultaneous, sequential or separate administration of the individual components of the treatment. In the field of medical oncology it is normal practice to use a combination of different forms of treatment to treat each patient with cancer. In medical oncology the other component(s) of such conjoint treatment in addition to the cell cycle inhibitory treatment defined hereinbefore may be: surgery, radiotherapy or chemotherapy. Such chemotherapy may cover three main categories of therapeutic agent:

(i) other cell cycle inhibitory agents that work by the same or different mechanisms from those defined hereinbefore;
(ii) cytostatic agents such as antioestrogens (for example tamoxifen, toremifene, raloxifene, droloxifene, iodoxyfene), progestogens (for example megestrol acetate), aromatase inhibitors (for example anastrozole, letrazole, vorazole, exemestane), antiprogestogens, antiandrogens (for example flutamide, nilutamide, bicalutamide, cyproterone acetate), LHRH agonists and antagonists (for example goserelin acetate, luprolide), inhibitors of testosterone 5α-dihydroreductase (for example finasteride), anti-invasion agents (for example metalloproteinase inhibitors like marimastat and inhibitors of urokinase plasminogen activator receptor function) and inhibitors of growth factor function, (such growth factors include for example platelet derived growth factor and hepatocyte growth factor such inhibitors include growth factor antibodies, growth factor receptor antibodies, tyrosine kinase inhibitors and serine/threonine kinase inhibitors); and
(iii) antiproliferative/antineoplastic drugs and combinations thereof, as used in medical oncology, such as antimetabolites (for example antifolates like methotrexate, fluoropyrimidines like 5-fluorouracil, purine and adenosine analogues, cytosine arabinoside); antitumour antibiotics (for example anthracyclines like doxorubicin, daunomycin, epirubicin and idarubicin, mitomycin-C, dactinomycin, mithramycin); platinum derivatives (for example cisplatin, carboplatin); alkylating agents (for example nitrogen mustard, melphalan, chlorambucil, busulphan, cyclophosphamide, ifosfamide, nitrosoureas, thiotepa); antimitotic agents (for example vinca alkaloids like vincristine and taxoids like taxol, taxotere); topoisomerase inhibitors (for example epipodophyllotoxins like etoposide and teniposide, amsacrine, topotecan). According to this aspect of the invention there is provided a pharmaceutical product comprising a compound of the formula (I) as defined hereinbefore and an additional anti-tumour substance as defined hereinbefore for the conjoint treatment of cancer.

In addition to their use in therapeutic medicine, the compounds of formula (I) and their pharmaceutically acceptable salts are also useful as pharmacological tools in the development and standardisation of in vitro and in vivo test systems for the evaluation of the effects of inhibitors of cell cycle activity in laboratory animals such as cats, dogs, rabbits, monkeys, rats and mice, as part of the search for new therapeutic agents.

In the above other pharmaceutical composition, process, method, use and medicament manufacture features, the alternative and preferred embodiments of the compounds of the invention described herein also apply.

### EXAMPLES

The invention will now be illustrated by the following non limiting examples in which, unless stated otherwise:

(i) temperatures are given in degrees Celsius (° C.); operations were carried out at room or ambient temperature, that is, at a temperature in the range of 18-25° C.;
(ii) organic solutions were dried over anhydrous magnesium sulphate; evaporation of solvent was carried out using a rotary evaporator under reduced pressure (600-4000 Pascals; 4.5-30 mmHg) with a bath temperature of up to 60° C.;
(iii) chromatography means flash chromatography on silica gel; thin layer chromatography (TLC) was carried out on silica gel plates;
(iv) in general, the course of reactions was followed by TLC and reaction times are given for illustration only;
(v) final products had satisfactory proton nuclear magnetic resonance (NMR) spectra and/or mass spectral data;
(vi) yields are given for illustration only and are not necessarily those which can be obtained by diligent process development; preparations were repeated if more material was required;
(vii) when given, NMR data is in the form of delta values for major diagnostic protons, given in parts per million (ppm) relative to tetramethylsilane (TMS) as an internal standard, determined at 300 MHz using perdeuterio dimethyl sulphoxide (DMSO-d₆) as solvent unless otherwise indicated;
(viii) chemical symbols have their usual meanings; SI units and symbols are used;
(ix) solvent ratios are given in volume:volume (v/v) terms; and
(x) mass spectra were run with an electron energy of 70 electron volts in the chemical ionization (CI) mode using a direct exposure probe; where indicated ionization was effected by electron impact (EI), fast atom bombardment (FAB) or electrospray (ESP); values for m/z are given; generally, only ions which indicate the parent mass are reported; and unless otherwise stated, the mass ion quoted is (MH)⁺;
(xi) unless stated otherwise compounds containing an asymmetrically substituted carbon and/or sulphur atom have not been resolved;
(xii) where a synthesis is described as being analogous to that described in a previous example the amounts used are the millimolar ratio equivalents to those used in the previous example;
(xvi) the following abbreviations have been used:
THF tetrahydrofuran;
DMF N,N-dimethylformamide;
EtOAc ethyl acetate;
MeOH methanol;
ether diethyl ether;
EtOH ethanol;
HATU O-(7-azabenzotriazol-1-yl)-1,1-3,3-tetramethyluronium hexafluorophosphate;
DCM dichloromethane;
cPr cyclopropyl;
RPHPLC reverse phase high performance liquid chromatography;
HBTU O-benzotriazol-1-yl-N,N,N′,N′-tetramethyluronium hexafluorophosphate;
DIPEA N,N-diisopropylethylamine;
DPPF 1,1′-bis(diphenylphosphino)ferrocene;
Pd₂(dba)₃ bis(dibenzylideneacetone)palladium;
TEA triethylamine;
DMFDMA N,N-dimethylformamide dimethyl acetal;
DMSO dimethylsulphoxide; and
XANTPHOS 9,9-dimethyl-4,5-bis(diphenylphosphino)xanthene.

### Example 1

### 4-[4-(3-Isopropyl-2-methyl-3H-imidazol-4-yl)-pyrimidin-2-ylamino]-3 N,N-trimethyl-benzamide

4-(3-Isopropyl-2-methyl-3H-imidazol-4-yl)-pyrimidin-2-ylamine (Method 18; 0.15 g, 0.69 mmol), Pd(OAc)2 (10 mg, 0.02 mmol), XANTPHOS (36 mg, 0.06 mmol), caesium carbonate (0.45 g, 1.38 mmol) and 4-bromo-3-methyl-N,N-dimethyl-benzamide (Intermediate 4 in GB2276160; 0.9 mmol, 218 mg) were pre-mixed in dioxane (5 ml) under a nitrogen atmosphere and the reaction was heated at reflux under nitrogen for 24 hours. The reaction was poured directly onto a column of silica gel and was eluted with DCM, 1.0% MeOH/DCM and finally 2.5% MeOH/DCM. A white foam was obtained (0.21 g, 81%). NMR (400.132 MHz, CDCl₃) 8.35 (d, 1H), 8.02 (d, 1H), 7.38 (s, 1H), 7.33 (s, 1H), 7.28 (d, 1H), 6.93 (d, 1H), 6.86 (s, 1H), 5.58 (septet, 1H), 3.07 (s, 6H), 2.57 (s, 3H), 2.35 (s, 3H), 1.44 (d, 6H); m/z 379.

### Examples 2-20

The following compounds were prepared by the procedure of Example 1 and on the same scale, using the appropriate amide starting material (method of preparation indicated if not commercially available) and 4-(3-isopropyl-2-methyl-3H-imidazol-4-yl)-pyrimidin-2-ylamine (Method 18).

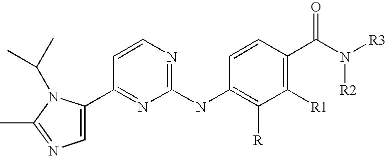

| Ex | R | R1 | R2 | R3 | NMR | m/z | Amide |
|---|---|---|---|---|---|---|---|
| 2 | H | F | Me | Me | (400.132 MHz, CDCl₃) 8.39 (d, 1 H), 7.76 (d, 1 H), 7.39 (s, 1 H), 7.36 (t, 2 H), 7.18 (d, | 383 | Method |
| | | | | | 1 H), 6.99 (d, 1 H), 5.64 (septet, 1 H), 3.12 (s, 3 H), 2.97 (s, 3 H), 2.60 (s, 3 H), 1.55 (d, | | 8 |
| | | | | | 6 H) | | |
| 3 | F | H | Me | Me | (400.132 MHz, CDCl₃) 8.49 (t, 1 H), 8.40 (d, 1 H), 7.39 (s, 1 H), 7.31 (s, 1 H), 7.27-7.22 | 383 | Method |
| | | | | | (m, 3 H), 6.99 (d, 1 H), 5.60 (septet, 1 H), 3.07 (s, 6 H), 2.60 (s, 3 H), 1.54 (d, 6 H) | | 63 |
| 4 | H | Cl | Me | Me | (400.132 MHz, CDCl₃) 8.39 (d, 1 H), 7.82 (s, 1 H), 7.45 (d, 1 H), 7.39 (s, 1 H), 7.34 (s, | 399 | Method |
| | | | | | 1 H), 7.26 (d, 1 H), 6.97 (d, 1 H), 5.59 (septet, 1 H), 3.14 (s, 3 H), 2.90 (s, 3 H), 2.60 (s, | | 11 |
| | | | | | 3 H), 1.54 (d, 6 H) | | |
| 5 | H | F | H | cPr | (400.132 MHz, CDCl₃) 8.41 (d, 1 H), 8.07 (t, 1 H), 7.84 (d, 1 H), 7.51 (s, 1 H), 7.40 (s, | 395 | Method |
| | | | | | 1 H), 7.21 (d, 1 H), 7.01 (d, 1 H), 6.77 (d, 1 H), 5.63 (septet, 1 H), 2.98-2.90 (m, 1 H), | | 10 |
| | | | | | 2.60 (s, 3 H), 1.56 (d, 6 H), 0.87 (q, 2 H), 0.65-0.61 (m, 2 H) | | |
| 6 | H | Me | Me | Me | (400.132 MHz, CDCl₃) 7.83 (d, 1 H), 7.03 (d, 1 H), 6.84 (s, 1 H), 6.82 (s, 1 H), 6.74 (s, | 379 | Method |
| | | | | | 1 H), 6.61 (d, 1 H), 6.39 (d, 1 H), 5.08 (septet, 1 H), 2.60 (s, 3 H), 2.34 (s, 3 H), 2.05 (s, | | 6 |
| | | | | | 3 H), 1.77 (s, 3 H), 0.98 (d, 6 H) | | |
| 7 | H | Cl | H | cPr | (400.132 MHz) 9.74 (s, 1 H), 8.47 (d, 1 H), 8.30 (s, 1 H), 7.91 (s, 1 H), 7.69 (d, 1 H), | 411 | Method |
| | | | | | 7.46 (s, 1 H), 7.35 (d, 1 H), 7.14 (d, 1 H), 5.63 (septet, 1 H), 2.83-2.79 (m, 1 H), 1.49 | | 12 |
| | | | | | (d, 6 H), 0.71-0.66 (m, 2 H), 0.55-0.51 (m, 2 H) | | |
| 8 | H | Me | H | Me | (400.132 MHz, CDCl₃) 8.36 (d, 1 H), 7.57 (d, 1 H), 7.35 (s, 1 H), 7.32 (d, 2 H), 7.27 (s, | 365 | Method |
| | | | | | 1 H), 6.92 (d, 1 H), 5.88 (s, 1 H), 5.59 (septet, 1 H), 2.99 (d, 3 H), 2.57 (s, 3 H), 2.47 (s, | | 7 |
| | | | | | 3 H), 1.51 (d, 6 H) | | |
| 9 | H | F | H | Me | (299.954 MHz, CDCl₃) 8.40 (d, 1 H), 8.08 (t, 1 H), 7.85 (d, 1 H), 7.40-7.38 (m, 2 H), | 369 | Method |
| | | | | | 7.20 (d, 1 H), 7.01 (d, 1 H), 6.69 (d, 1 H), 5.63 (septet, 1 H), 3.03 (d, 3 H), 2.60 (s, 3 H), | | 9 |
| | | | | | 1.56 (d, 6 H) | | |
| 10 | H | Cl | H | H | (299.955 MHz) 9.72 (s, 1 H), 8.45 (d, 1 H), 7.88 (s, 1 H), 7.69-7.66 (m, 2 H), 7.44-7.40 | 371 | Method |
| | | | | | (m, 3 H), 7.12-7.11 (d, 1 H), 5.61 (septet, 1 H), 1.47 (d, 6 H) | | 5 |
| 11 | H | F | H | H | (400.132 MHz) 9.92 (s, 1 H), 8.49 (d, 1 H), 7.80 (d, 1 H), 7.69 (t, 1 H), 7.53 (d, 1 H), | 355 | Method |
| | | | | | 7.47 (s, 1 H), 7.45 (brs, 1 H), 7.38 (brs, 1 H), 7.17 (d, 1 H), 5.68 (septet, 1 H), 1.50 (d, | | 4 |
| | | | | | 6 H) | | |
| 12 | H | Me | H | H | (299.954 MHz, CDCl₃) 8.37 (d, 1 H), 7.62 (d, 1 H), 7.47 (d, 1 H), 7.39-7.33 (m, 3 H), | 351 | Method |
| | | | | | 6.94 (d, 1 H), 5.85 (brs, 2 H), 5.59 (septet, 1 H), 2.58 (s, 3 H), 2.53 (s, 3 H), 1.53 (d, 6 H) | | 13 |
| 13 | H | H | H | Me | (400.132 MHz, CDCl₃) 8.38 (d, 1 H), 7.76 (d, 2 H), 7.67 (d, 2 H), 7.42 (s, 1 H), 7.38 (s, | 351 | Method |
| | | | | | 1 H), 6.96 (d, 1 H), 6.22 (brs, 1 H), 5.65 (septet, 1 H), 3.01 (d, 3 H), 2.59 (s, 3 H), 1.53 | | 2 |
| | | | | | (d, 6 H) | | |
| 14 | H | H | Me | Me | (400.132 MHz, CDCl₃) 8.37 (d, 1 H), 7.65 (d, 2 H), 7.43 (d, 2 H), 7.38 (s, 1 H), 7.13 (s, | 365 | Method |
| | | | | | 1 H), 6.95 (d, 1 H), 5.65 (septet, 1 H), 3.07 (s, 6 H), 2.59 (s, 3 H), 1.53 (d, 6 H) | | 1 |
| 15 | H | H | H | cPr | (400.132 MHz, CDCl₃) 8.38 (d, 1 H), 7.73 (d, 2 H), 7.67 (d, 2 H), 7.38 (s, 1 H), 7.34 (s, | 377 | Method |
| | | | | | 1 H), 6.96 (d, 1 H), 6.25 (brs, 1 H), 5.64 (septet, 1 H), 2.94-2.88 (m, 1 H), 2.59 (s, 3 H), | | 3 |
| | | | | | 1.54 (d, 6 H), 0.87 (q, 2 H), 0.64-0.60 (m, 2 H) | | |
| 16 | H | Me | H | cPr | (400.132 MHz) 9.49 (s, 1 H), 8.42 (d, 1 H), 8.12 (s, 1 H), 7.64 (d, 1 H), 7.49 (s, 1 H), | 491 | Method |
| | | | | | 7.43 (s, 1 H), 7.26 (d, 1 H), 7.07 (d, 1 H), 5.68 (septet, 1 H), 2.85-2.78 (m, 1 H), 2.50 (s, | | 22 |
| | | | | | 3 H), 2.34 (s, 3 H), 1.47 (d, 6 H), 0.70-0.65 (m, 2 H), 0.55-0.51 (m, 2 H) | | |
| 17 | H | H | H | 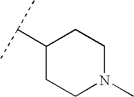 | (400.13 MHz) 9.74 (s, 1 H), 8.45 (d, 1 H), 8.06 (d, 1 H), 7.82 (d, 2 H), 7.78 (d, 2 H),7.46 (s, 1 H), 7.12 (d, 1 H), 5.79-5.67 (m, 1 H), 3.82-3.68 (m, 1 H), 2.78 (d, 2 H), 2.52 (s,3 H overlapping water), 2.17 (s, 3 H), 1.94 (t, 2 H), 1.75 (d, 2 H), 1.65-1.52 (m, 2 H),1.49 (d, 6 H) | 434 | Method29 |
| 18 | H | H | H | 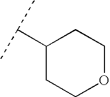 | (400.13 MHz) 9.75 (s, 1 H), 8.45 (d, 1 H), 8.13 (d, 1 H), 7.82 (d, 2 H), 7.79 (d, 2 H),7.47 (s, 1 H), 7.13 (d, 1 H), 5.79-5.67 (m, 1 H), 4.06-3.94 (m, 1 H), 3.89 (d, 2 H),3.44-3.30 (m, 5 H), 2.09 (d, 2 H), 1.65-1.52 (m, 2 H), 1.49 (d, 6 H) | 421 | Method30 |
| 19 | H | F | H | 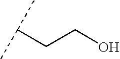 | (400.132 MHz, CDCl₃) 8.40 (d, 1 H), 8.05 (t, 1 H), 7.85 (d, 1 H), 7.45 (s, 1 H), 7.38 (s,1 H), 7.21 (d, 1 H), 7.14-7.10 (m, 1 H), 7.00 (d, 1 H), 5.62 (septet, 1 H), 3.85 (t, 2 H),3.66 (q, 2 H), 2.60 (s, 3 H), 1.70 (brs 1 H), 1.56 (d, 6 H) | 399 | Method14 |
| 20 | H | H | H | 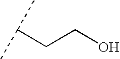 | (400.132 MHz, CDCl₃) 8.36 (d, 1 H), 7.76 (d, 2 H), 7.68 (d, 2 H), 7.63 (s, 1 H), 7.38 (s,1 H), 6.94 (d, 1 H), 6.72 (s, 1 H), 5.64 (septet, 1 H), 4.06 (s, 1 H), 3.85 (t, 2 H), 3.64 (q,2 H), 2.59 (s, 3 H), 1.53 (d, 6 H) | 381 | Method15 |

### Example 21

### 2-Fluoro-4-[5-fluoro-4-(3-isopropyl-2-methyl-3H-imidazol-4-yl)-pyrimidin-2-ylamino]-N,N-dimethyl-benzamide

5-Fluoro-4-(3-isopropyl-2-methyl-3H-imidazol-4-yl)-pyrimidin-2-ylamine (Method 17; 0.15 g, 0.69 mmol), Pd(OAc)2 (10 mg, 0.02 mmol), XANTPHOS (36 mg, 0.06 mmol), caesium carbonate (0.45 g, 1.38 mmol) and 4-bromo-2-fluoro-N,N-dimethyl-benzamide (Method 8; 0.70 mmol, 173 mg) were pre-mixed in dioxane (5 ml) under a nitrogen atmosphere and the reaction was heated at reflux under nitrogen for 24 hours. The reaction was poured directly onto a column of silica gel and was eluted with DCM, 1.0% MeOH/DCM and finally 2.5% MeOH/DCM. White foam obtained (150 mg, 59%). NMR (400.132 MHz, CDCl₃) 8.32 (d, 1H), 7.68 (d, 1H), 7.60 (d, 1H), 7.37-7.33 (m, 2H), 7.16 (d, 1H), 5.56 (septet, 1H), 3.12 (s, 3H), 2.97 (s, 3H), 2.62 (s, 3H), 1.54 (d, 6H); m/z 401.

### Examples 22-55

The following compounds were prepared by the procedure of Example 21 and on the same scale, using the appropriate amide starting material (method of preparation indicated if not commercially available) and the appropriate amine.

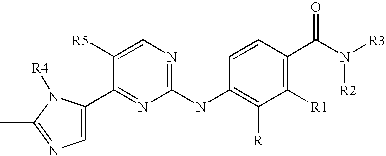

| Ex | R | R1 | R2 | R3 | R4 | R5 | NMR | m/z | Amide |
|---|---|---|---|---|---|---|---|---|---|
| 22 | H | Cl | Me | Me | iPr | F | (400.132 MHz, CDCl₃) 7.79 (d, 1 H), 7.23 (s, 1 H), 7.07 (d, | 417 | Method 11 and |
| | | | | | | | 1 H), 6.89 (d, 1 H), 6.74-6.70 (m, 2 H), 4.99 (septet, 1 H), | | Method 17 |
| | | | | | | | 2.61 (s, 3 H), 2.38 (s, 3 H), 2.09 (s, 3 H), 1.01 (d, 6 H) | | |
| 23 | H | Me | Me | Me | iPr | F | (400.132 MHz, CDCl₃) 8.29 (d, 1 H), 7.58 (d, 1 H), 7.50 (d, | 397 | Method 6 and |
| | | | | | | | 1 H), 7.31 (s, 1 H), 7.14 (d, 1 H), 7.10 (s, 1 H), 5.53 (septet, | | Method 17 |
| | | | | | | | 1 H), 3.13 (s, 3 H), 2.87 (s, 3 H), 2.61 (s, 3 H), 2.29 (s, 3 H), | | |
| | | | | | | | 1.51 (d, 6 H) | | |
| 24 | Me | H | Me | Me | iPr | F | (299.954 MHz, cdcl3) δ 8.27 (d, 1 H), 7.93 (d, 1 H), 7.59 (d, | 397 | Method 17 and |
| | | | | | | | 1 H), 7.34-7.32 (m, 1 H), 7.30-7.25 (m, 1 H), 6.83 (s, 1 H), | | Intermediate 4 |
| | | | | | | | 5.51 (septet, 1 H), 3.07 (s, 6 H), 2.58 (s, 3 H), 2.33 (s, 3 H), | | in GB2276160 |
| | | | | | | | 1.43 (d, 6 H) | | |
| 25 | H | F | H | cPr | iPr | F | (400.132 MHz) 8.64 (d, 1 H), 8.03 (s, 1 H), 7.72 (d, 1 H), | 413 | Method 10 and |
| | | | | | | | 7.55 (t, 1 H), 7.46 (d, 1 H), 7.39 (d, 1 H), 5.42 (septet, 1 H), | | Method 17 |
| | | | | | | | 2.86-2.79 (m, 1 H), 2.54 (s, 3 H), 1.48 (d, 6 H), 0.69 (q, | | |
| | | | | | | | 2 H), 0.57-0.53 (m, 2 H) | | |
| 26 | F | H | Me | Me | iPr | F | (400.132 MHz, CDCl₃) 8.39 (t, 1 H), 8.33 (d, 1 H), 7.60 (d, | 401 | Method 17 |
| | | | | | | | 1 H), 7.30-7.22 (m, 3 H), 5.52 (septet, 1 H), 3.07 (s, 6 H), | | Method 63 |
| | | | | | | | 2.62 (s, 3 H), 1.54 (d, 6 H) | | |
| 27 | H | Cl | H | cPr | iPr | F | (400.132 MHz) 8.63 (d, 1 H), 8.29 (d, 1 H), 7.86 (d, 1 H), | 429 | Method 12 and |
| | | | | | | | 7.64 (d, 1 H), 7.38 (d, 1 H), 7.34 (d, 1 H), 5.38 (septet, 1 H), | | Method 17 |
| | | | | | | | 2.84-2.77 (m, 1 H), 2.54 (s, 3 H), 1.48 (d, 6 H), 0.71-0.66 | | |
| | | | | | | | (m, 2 H), 0.55-0.51 (m, 2 H) | | |
| 28 | H | Me | H | Me | iPr | F | (400.132 MHz, CDCl₃) 8.29 (d, 1 H), 7.57 (d, 1 H), 7.52 (d, | 383 | Method 7 and |
| | | | | | | | 1 H), 7.34 (d, 1 H), 7.27 (s, 1 H), 7.14 (s, 1 H), 5.83 (s, 1 H), | | Method 17 |
| | | | | | | | 5.51 (septet, 1 H), 2.99 (d, 3 H), 2.60 (s, 3 H), 2.47 (s, 3 H), | | |
| | | | | | | | 1.52 (d, 6 H) | | |
| 29 | H | F | H | Me | iPr | F | (400.132 MHz) 10.01 (s, 1 H), 8.65 (d, 1 H), 7.92 (t, 1 H), | 387 | Method 9 and |
| | | | | | | | 7.74 (d, 1 H), 7.64 (t, 1 H), 7.48 (d, 1 H), 7.40 (d, 1 H), 5.42 | | Method 17 |
| | | | | | | | (septet, 1 H), 2.78 (d, 3 H), 2.55 (s, 3 H), 1.48 (d, 6 H) | | |
| 30 | H | Cl | H | H | iPr | F | (400.132 MHz) 9.90 (s, 1 H), 8.68 (d, 1 H), 7.90 (s, 1 H), | 389 | Method 5 and |
| | | | | | | | 7.74 (s, 1 H), 7.70 (d, 1 H), 7.49-7.47 (m, 2 H), 7.44 (d, | | Method 17 |
| | | | | | | | 1 H), 5.43 (septet, 1 H), 2.59 (s, 3 H), 1.53 (d, 6 H) | | |
| 31 | H | F | H | H | iPr | F | (400.132 MHz) 10.03 (s, 1 H), 8.66 (d, 1 H), 7.73 (d, 1 H), | 373 | Method 4 and |
| | | | | | | | 7.69 (t, 1 H), 7.49 (d, 1 H), 7.43-7.42 (m, 2 H), 7.37 (s, 1 H), | | Method 17 |
| | | | | | | | 5.42 (septet, 1 H), 2.56 (s, 3 H), 1.49 (d, 6 H) | | |
| 32 | H | Me | H | H | iPr | F | (400.132 MHz) 9.60 (s, 1 H), 8.58 (s, 1 H), 7.59 (d, 1 H), | 369 | Method 13 and |
| | | | | | | | 7.54 (brs, 1 H), 7.43 (s, 1 H), 7.38 (d, 2 H), 7.13 (s, 1 H), 5.43 | | Method 17 |
| | | | | | | | (septet, 1 H), 2.53 (s, 3 H), 2.38 (s, 3 H), 1.46 (d, 6 H) | | |
| 33 | H | Cl | H | Me | iPr | F | (299.954 MHz, CDCl₃) 8.32 (d, 1 H), 7.79-7.76 (m, 2 H), | 404 | Method 19 and |
| | | | | | | | 7.60 (d, 1 H), 7.43 (d, 1 H), 7.15 (s, 1 H), 6.43 (s, 1 H), 5.50 | | Method 17 |
| | | | | | | | (septet, 1 H), 3.03 (d, 3 H), 2.62 (s, 3 H), 1.55 (d, 6 H) | | |
| 34 | H | H | H | Me | iPr | F | (400.132 MHz) 9.81 (s, 1 H), 8.60 (d, 1 H), 8.26-8.22 (m, | 369 | Method 2 and |
| | | | | | | | 1 H), 7.79 (d, 2 H), 7.73 (d, 2 H), 7.39 (d, 1 H), 5.48 (septet, | | Method 17 |
| | | | | | | | 1 H), 2.78 (d, 3 H), 2.54 (s, 3 H), 1.47 (d, 6 H) | | |
| 35 | H | H | Me | Me | iPr | F | (400.132 MHz, CDCl₃) 8.30 (d, 1 H), 7.60-7.58 (m, 3 H), | 383 | Method 1 and |
| | | | | | | | 7.43 (d, 2 H), 7.17 (s, 1 H), 5.58 (septet, 1 H), 3.07 (s, 6 H), | | Method 17 |
| | | | | | | | 2.61 (s, 3 H), 1.53 (d, 6 H) | | |
| 36 | H | H | H | cPr | iPr | F | (400.132 MHz) 9.80 (s, 1 H), 8.60 (d, 1 H), 8.23 (d, 1 H), | 395 | Method 3 and |
| | | | | | | | 7.78 (d, 2 H), 7.73 (d, 2 H), 7.39 (d, 1 H), 5.47 (septet, 1 H), | | Method 17 |
| | | | | | | | 3.29 (s, 3 H), 2.86-2.79 (m, 1 H), 2.54 (s, 3 H), 1.47 (d, | | |
| | | | | | | | 6 H), 0.71-0.67 (m, 2 H), 0.58-0.54 (m, 2 H) | | |
| 37 | H | Me | H | cPr | iPr | F | (400.132 MHz) 9.59 (s, 1 H), 8.57 (d, 1 H), 8.10 (d, 1 H), | 409 | Method 22 and |
| | | | | | | | 7.59 (d, 1 H), 7.44 (s, 1 H), 7.37 (d, 1 H), 7.25 (d, 1 H), 5.42 | | Method 17 |
| | | | | | | | (septet, 1 H), 2.84-2.78 (m, 1 H), 2.53 (s, 3 H), 2.33 (s, 3 H), | | |
| | | | | | | | 1.46 (d, 6 H), 0.69-0.65 (m, 2 H), 0.54-0.50 (m, 2 H) | | |
| 38 | H | H | H | cPr | Et | F | 9.70 (s, 1 H), 8.54 (d, 1 H), 8.22 (d, 1 H), 7.77 (d, 2 H), 7.68 | 381 | Method 26 and |
| | | | | | | | (d, 2 H), 7.58 (d, 1 H), 4.58 (q, 2 H), 2.85-2.78 (m, 1 H), | | Method 3 |
| | | | | | | | 2.43 (s, 3 H), 1.17 (t, 3 H), 0.70-0.64 (m, 2 H), 0.58-0.53 | | |
| | | | | | | | (m, 2 H) | | |
| 39 | H | H | H | cPr | cBu | F | 9.86 (s, 1 H), 8.58 (d, 1 H), 8.22 (d, 1 H), 7.80-7.73 (m, | 407 | Method 28 and |
| | | | | | | | 4 H), 7.32 (d, 1 H), 5.27 (quin., 1 H), 2.85-2.78 (m, 1 H), | | Method 3 |
| | | | | | | | 2.49 (s, 3 H), 2.40-2.30 (m, 4 H), 1.72-1.57 (m, 2 H), 0.70-0.64 | | |
| | | | | | | | (m, 2 H), 0.57-0.53 (m, 2 H) | | |
| 40 | H | F | Me | H | Et | H | (400.132 MHz) 9.87 (s, 1 H), 8.45 (d, 1 H), 7.99-7.97 (m, | 355 | Method 30 of |
| | | | | | | | 1 H), 7.81 (d, 1 H), 7.71 (s, 1 H), 7.65 (t, 1 H), 7.50 (d, 1 H), | | WO 02/020512 |
| | | | | | | | 7.25 (d, 1 H), 4.60 (q, 2 H), 2.78 (d, 3 H), 2.42 (s, 3 H), 1.22 | | and Method 9 |
| | | | | | | | (t, 3 H) | | |
| 41 | H | Me | Me | H | Et | H | (400.132 MHz, CDCl₃) 8.34 (d, 1 H), 7.52 (s, 1 H), 7.49 (d, | 351 | Method 30 of |
| | | | | | | | 1 H), 7.37-7.34 (m, 2 H), 7.20 (s, 1 H), 6.96 (d, 1 H), 5.92-5.85 | | WO 02/020512 |
| | | | | | | | (m, 1 H), 4.50 (q, 2 H), 3.00 (d, 3 H), 2.48 (s, 3 H), 2.46 | | and Method 7 |
| | | | | | | | (s, 3 H), 1.27 (t, 3 H) | | |
| 42 | H | H | Me | H | Et | H | (400.132 MHz) 9.61 (s, 1 H), 8.35 (d, 1 H), 8.21 (q, 1 H), | 337 | Method 30 of |
| | | | | | | | 7.73 (d, 2 H), 7.69 (d, 2 H), 7.62 (s, 1 H), 7.13 (d, 1 H), 4.53 | | WO 02/020512 |
| | | | | | | | (q, 2 H), 2.71 (d, 3 H), 2.34 (s, 3 H), 1.13 (t, 3 H) | | and Method 2 |
| 43 | H | H | Me | H | cBu | H | (400.132 MHz, CDCl₃) 8.39 (d, 1 H), 7.78 (d, 2 H), 7.73 (s, | 363 | Method 51 of |
| | | | | | | | 1 H), 7.70 (d, 2 H), 7.31 (s, 1 H), 6.93 (d, 1 H), 6.39-6.38 (q, | | WO 03/076435 |
| | | | | | | | 1 H), 5.39 (quintet, 1 H), 3.02 (d, 3 H), 2.57 (s, 3 H), 2.44 (q, | | and Method 2 |
| | | | | | | | 4 H), 1.83-1.66 (m, 2 H) | | |
| 44 | H | F | Me | H | cBu | H | (400.132 MHz, CDCl₃) 8.42 (d, 1 H), 8.09 (t, 1 H), 7.91 (d, | 381 | Method 51 of |
| | | | | | | | 1 H), 7.72 (s, 1 H), 7.33 (s, 1 H), 7.25 (d, 1 H), 6.98 (d, 1 H), | | WO 03/076435 |
| | | | | | | | 6.77-6.72 (m, 1 H), 5.40 (quintet, 1 H), 3.04 (d, 3 H), 2.61 | | and Method 9 |
| | | | | | | | (s, 3 H), 2.50-2.43 (m, 4 H), 1.85-1.71 (m, 2 H) | | |
| 45 | H | H | Me | H | cPr- | H | (400.132 MHz) 9.56 (s, 1 H), 8.26 (d, 1 H), 8.12 (q, 1 H), | 363 | Method 54 of |
| | | | | | CH₂— | | 7.64 (d, 2 H), 7.59 (d, 2 H), 7.50 (s, 1 H), 7.03 (d, 1 H), 4.41 | | WO 03/076435 |
| | | | | | | | (d, 2 H), 2.62 (d, 3 H), 2.26 (s, 3 H), 0.94-0.84 (m, 1 H), | | and Method 2 |
| | | | | | | | 0.19-0.14 (m, 2 H), −0.01 (q, 2 H) | | |
| 46 | H | F | Me | H | cPr- | H | (400.132 MHz) 9.72 (s, 1 H), 8.27 (d, 1 H), 7.82-7.74 (m, | 381 | Method 54 of |
| | | | | | CH₂— | | 1 H), 7.61 (d, 1 H), 7.49-7.44 (m, 2 H), 7.31 (d, 1 H), 7.06 | | WO 03/076435 |
| | | | | | | | (d, 1 H), 4.39 (d, 2 H), 2.59 (d, 3 H), 2.23 (s, 3 H), 0.94-0.84 | | and Method 9 |
| | | | | | | | (m, 1 H), 0.19-0.14 (m, 2 H), −0.01 (q, 2 H) | | |
| 47 | H | F | Me | H | cPr- | H | (400.132 MHz) 9.88 (s, 1 H), 8.51 (d, 1 H), 8.03-8.01 (m, | 395 | Method 57 of |
| | | | | | CH(Me)- | | 1 H), 7.79 (d, 1 H), 7.69 (t, 1 H), 7.56 (s, 1 H), 7.52 (d, 1 H), | | WO 03/076435 |
| | | | | | | | 7.23 (d, 1 H), 4.97-4.84 (m, 1 H), 2.82 (d, 3 H), 2.60 (s, | | and Method 9 |
| | | | | | | | 3 H), 1.67 (d, 3 H), 1.55-1.46 (m, 1 H), 0.65-0.58 (m, 1 H), | | |
| | | | | | | | 0.42-0.27 (m, 2 H), 0.02--0.04 (m, 1 H) | | |
| 48 | H | H | Me | H | cPr- | H | (400.132 MHz) 9.72 (s, 1 H), 8.48 (d, 1 H), 8.35 (q, 1 H), | 377 | Method 57 of |
| | | | | | CH(Me)- | | 7.86 (d, 2 H), 7.76 (d, 2 H), 7.57 (s, 1 H), 7.19 (d, 1 H), 5.06-4.93 | | WO 03/076435 |
| | | | | | | | (m, 1 H), 2.84 (d, 3 H), 2.61 (s, 3 H), 1.66 (d, 3 H), 1.55-1.46 | | and Method 2 |
| | | | | | | | (m, 1 H), 0.63-0.56 (m, 1 H), 0.43-0.36 (m, 1 H), | | |
| | | | | | | | 0.28-0.20 (m, 1 H), 0.02--0.04 (m, 1 H) | | |
| 49 | H | F | HO—(CH₂)₂— | H | iPr | F | (400.132 MHz, CDCl₃) 7.91 (d, 1 H), 7.63 (t, 1 H), 7.35 (d, | 417 | Method 14 and |
| | | | | | | | 1 H), 7.17 (d, 1 H), 6.79 (s, 1 H), 6.75 (d, 1 H), 6.68-6.64 | | Method 17 |
| | | | | | | | (m, 1 H), 5.11 (septet, 1 H), 3.42 (q, 2 H), 3.23 (q, 2 H), | | |
| | | | | | | | 2.22-2.20 (m, 4 H), 1.13 (d, 6 H) | | |
| 50 | H | H | HO—(CH₂)₂— | H | iPr | F | (400.132 MHz, CDCl₃) 8.30 (d, 1 H), 7.77 (d, 2 H), 7.63 (d, | 399 | Method 15 and |
| | | | | | | | 2 H), 7.58 (d, 1 H), 6.64 (brs, 1 H), 5.55 (septet, 1 H), 3.85 (t, | | Method 17 |
| | | | | | | | 2 H), 3.64 (q, 2 H), 3.15 (brs, 1 H) 2.61 (s, 3 H), 1.53 (d, 6 H) | | |
| 51 | H | F | 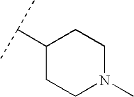 | H | iPr | F | (400.132 MHz) 10.17 (s, 1 H), 8.80 (d, 1 H), 8.03 (dd, 1 H),7.88 (dd, 1 H), 7.69 (t, 1 H), 7.60 (dd, 1 H), 7.53 (d, 1 H),5.56 (septet, 1 H), 3.88-3.78 (m, 1 H), 2.86 (d, 2 H), 2.68 (s,3 H), 2.29 (s, 3 H), 2.08 (t, 2 H), 1.92-1.86 (m, 2 H), 1.72-1.64(m, 2 H), 1.62 (d, 6 H) | 470 | Method 59 andMethod 17 |
| 52 | H | H | 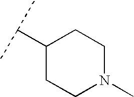 | H | cBu- | H | (490.132 MHz, CDCl3) 8.39 (d, 1 H), 7.73 (q, 4 H), 7.43 (s,1 H), 7.31 (s, 1 H), 6.93 (d, 1 H), 5.95 (d, 1 H), 5.40 (quintet,1 H), 4.05-3.95 (m, 1 H), 2.82 (d, 2 H), 2.59 (s, 3 H), 2.50-2.43(m, 4 H), 2.30 (s, 3 H), 2.17 (t, 2 H), 2.05 (d, 2 H), 1.85-1.69(m, 2 H), 1.59 (q, 2 H) | 447 | Method 29 andMethod 51 inWO03/076435 |
| 53 | H | F | 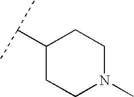 | H | cBu- | H | (400.132 MHz, CDCl3) 8.41 (d, 1 H), 8.05 (t, 1 H), 7.88 (d,1 H), 7.37 (s, 1 H), 7.32 (s, 1 H), 7.24 (d, 1 H), 6.97 (d, 1 H),6.62-6.56 (m, 1 H), 5.39 (quintet, 1 H), 4.10-4.01 (m,1 H), 2.87 (d, 2 H), 2.61 (s, 3 H), 2.51-2.44 (m, 4 H), 2.36(s, 3 H), 2.26 (t, 2 H), 2.08 (d, 2 H), 1.86-1.74 (m, 2 H), 1.67(q, 2 H) | 465 | Method 59 andMethod 51 inWO03/076435 |
| 54 | H | H | 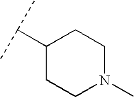 | H | cBu- | F | (400.132 MHz, CDCl3) 8.30 (d, 1 H), 7.76 (d, 2 H), 7.67 (d,2 H), 7.50 (d, 1 H), 7.34 (s, 1 H), 5.94 (d, 1 H), 5.28 (quintet,1 H), 4.05-3.95 (m, 1 H), 2.83-2.80 (m, 2 H), 2.59 (s, 3 H),2.48-2.35 (m, 4 H), 2.30 (s, 3 H), 2.17 (t, 2 H), 2.09-2.01(m, 2 H), 1.84-1.69 (m, 2 H), 1.58 (ddd, 2 H) | 464 | Method 28 andMethod 29 |
| 55 | H | H | Me | H | cBu- | F | (400.132 MHz) 9.87 (s, 1 H), 8.59 (d, 1 H), 8.22 (q, 1 H), | 381 | Method 28 and |
| | | | | | | | 7.81 (d, 2 H), 7.78 (d, 2 H), 7.34 (d, 1 H), 5.31 (quintet, 1 H), | | Method 2 |
| | | | | | | | 2.78 (d, 3 H), 2.53 (s, 3 H), 2.44-2.31 (m, 4 H), 1.77-1.58 | | |
| | | | | | | | (m, 2 H) | | |

### Examples 56-74

The following compounds were prepared by the procedure of Example 21 and on the same scale, using the appropriate amide starting material (method of preparation indicated if not commercially available) and the appropriate amine.

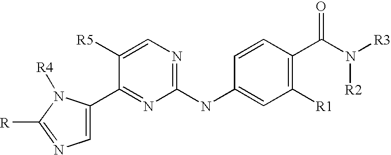

| Ex | R | R1 | R2 | R3 | R4 | R5 | NMR | m/z | Amide |
|---|---|---|---|---|---|---|---|---|---|
| 56 | Et | F | H | Me | cPr- | H | (400.132 MHz) 9.74 (s, 1 H), 8.29 (d, 1 H), 7.80-7.78 (m, 1 H), 7.62 | 395 | Method 9 and |
| | | | | | CH₂— | | (d, 1 H), 7.52 (s, 1 H), 7.47 (t, 1 H), 7.32 (d, 1 H), 7.07 (d, 1 H), 4.40 | | Method 56 in |
| | | | | | | | (d, 2 H), 2.60-2.56 (m, 5 H), 1.10 (t, 3 H), 0.93-0.79 (m, 1 H), | | WO03/076435 |
| | | | | | | | 0.18-0.13 (m, 2 H), 0.01--0.02 (m, 2 H) | | |
| 57 | Et | H | H | Me | cPr- | H | (400.132 MHz) 9.57 (s, 1 H), 8.27 (d, 1 H), 8.15-8.10 (m, 1 H), 7.65 | 377 | Method 2 and |
| | | | | | CH₂— | | (d, 2 H), 7.60 (d, 2 H), 7.53 (s, 1 H), 7.04 (d, 1 H), 4.42 (d, 2 H), 2.63-2.57 | | Method 56 in |
| | | | | | | | (m, 5 H), 1.12 (t, 3 H), 0.93-0.83 (m, 1 H), 0.19-0.15 (m, 2 H), | | WO03/076435 |
| | | | | | | | 0.02--0.03 (m, 2 H) | | |
| 58 | MeOCH₂— | F | H | Me | iPr- | H | (400.132 MHz) 9.99 (s, 1 H), 8.55 (d, 1 H), 7.99-7.97 (m, 1 H), 7.84 | 399 | Method 9 and |
| | | | | | | | (d, 1 H), 7.64 (t, 1 H), 7.52-7.50 (m, 2 H), 7.21 (d, 1 H), 5.53 (septet, | | Method 60 |
| | | | | | | | 1 H), 4.58 (s, 2 H), 3.31 (s, 3 H), 2.78 (d, 3 H), 1.52 (d, 6 H) | | |
| 59 | MeOCH₂— | H | H | Me | iPr- | H | (400.132 MHz) 9.80 (s, 1 H), 8.51 (d, 1 H), 8.28 (q, 1 H), 7.79 (s, 4 H), | 381 | Method 2 and |
| | | | | | | | 7.51 (s, 1 H), 7.16 (d, 1 H), 5.58 (septet, 1 H), 4.58 (s, 2 H), 3.31 (s, | | Method 60 |
| | | | | | | | 3 H), 2.78 (d, 3 H), 1.51 (d, 6 H) | | |
| 60 | iPr- | H | H | Me | Et | H | (400.132 MHz) 9.69 (s, 1 H), 8.42 (d, 1 H), 8.27 (q, 1 H), 7.80 (d, | 365 | Method 2 and |
| | | | | | | | 2 H), 7.76 (d, 2 H), 7.70 (s, 1 H), 7.20 (d, 1 H), 4.63 (q, 2 H), 3.16 | | Method 35 |
| | | | | | | | (septet, 1 H), 2.78 (d, 3 H), 1.27 (d, 6 H), 1.19 (t, 3 H) | | |
| 61 | iPr- | F | H | Me | Et | H | (400.132 MHz, CDCl3) 8.39 (d, 1 H), 8.07 (t, 1 H), 7.85 (d, 1 H), 7.65 | 383 | Method 9 and |
| | | | | | | | (s, 1 H), 7.61 (s, 1 H), 7.21 (d, 1 H), 7.05 (d, 1 H), 6.77-6.73 (m, 1 H), | | Method 35 |
| | | | | | | | 4.55 (q, 2 H), 3.15-3.03 (m, 4 H), 1.39 (d, 6 H), 1.32 (t, 3 H) | | |
| 62 | iPr- | H | H | 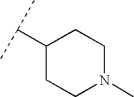 | Et | H | (400.132 MHz) 9.69 (s, 1 H), 8.42 (d, 1 H), 8.09 (d, 1 H), 7.83 (d,2 H), 7.77 (d, 2 H), 7.70 (s, 1 H), 7.19 (d, 1 H), 4.63 (q, 2 H), 3.80-3.71(m, 1 H), 3.17 (septet, 1 H), 2.85-2.82 (m, 2 H), 2.22 (t, 3 H),2.05 (t, 2 H), 1.79-1.77 (m, 2 H), 1.61 (q, 2 H), 1.27 (d, 6 H), 1.20 (t,3 H) | 448 | Method 29 andMethod 35 |
| 63 | cPr- | H | H | Me | Et | H | (400.132 MHz) 9.68 (s, 1 H), 8.41 (d, 1 H), 8.28 (q, 1 H), 7.81 (d, | 363 | Method 2 and |
| | | | | | | | 2 H), 7.77 (d, 2 H), 7.64 (s, 1 H), 7.18 (d, 1 H), 4.76 (q, 2 H), 2.78 (d, | | Method 40 |
| | | | | | | | 3 H), 2.14-2.08 (m, 1 H), 1.27 (t, 3 H), 1.01-0.96 (m, 2 H), 0.93-0.90 | | |
| | | | | | | | (m, 2 H) | | |
| 64 | cPr- | F | H | Me | Et | H | (400.132 MHz, CDCl3) 8.37 (d, 1 H), 8.08 (t, 1 H), 7.85 (d, 1 H), 7.55-7.52 | 381 | Method 9 and |
| | | | | | | | (m, 2 H), 7.21 (d, 1 H), 7.02 (d, 1 H), 6.77-6.73 (m, 1 H), 4.68 | | Method 40 |
| | | | | | | | (q, 2 H), 3.04 (d, 3 H), 1.92-1.86 (m, 1 H), 1.39 (t, 3 H), 1.14-1.09 | | |
| | | | | | | | (m, 2 H), 1.07-1.02 (m, 2 H) | | |
| 65 | cPr- | H | H | 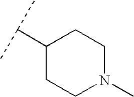 | Et | H | (400.132 MHz, CDCl3) 8.35 (d, 1 H), 7.76 (d, 2 H), 7.67 (d, 2 H),7.50 (s, 1 H), 6.98 (d, 1 H), 5.95 (d, 1 H), 4.68 (q, 2 H), 4.05-3.96 (m,1 H), 2.85-2.82 (m, 2 H), 2.31 (s, 3 H), 2.17 (t, 2 H), 2.07-2.05 (m,2 H), 1.92-1.85 (m, 2 H), 1.59 (q, 2 H), 1.37 (t, 3 H), 1.13-1.09 (m,2 H), 1.07-1.02 (m, 2 H) | 446 | Method 29 andMethod 40 |
| 66 | CF₃— | H | H | Me | Et | H | (400.132 MHz) 9.89 (s, 1 H), 8.60 (d, 1 H), 8.29 (q, 1 H), 7.96 (s, 1 H), | 391 | Method 2 and |
| | | | | | | | 7.82 (d, 2 H), 7.76 (d, 2 H), 7.37 (d, 1 H), 4.76 (q, 2 H), 2.78 (d, 3 H), | | Method 45 |
| | | | | | | | 1.27 (t, 3 H) | | |
| 67 | CF₃— | F | H | Me | Et | H | (400.132 MHz) 10.07 (s, 1 H), 8.65 (d, 1 H), 8.01-7.99 (m, 1 H), 7.98 | 409 | Method 9 and |
| | | | | | | | (s, 1 H), 7.81 (d, 1 H), 7.67 (t, 1 H), 7.51 (d, 1 H), 7.43 (d, 1 H), 4.77 | | Method 45 |
| | | | | | | | (q, 2 H), 2.78 (d, 3 H), 1.29 (t, 3 H) | | |
| 68 | CF₃— | H | H | 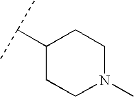 | Et | H | (400.132 MHz) 9.89 (s, 1 H), 8.60 (d, 1 H), 8.08 (d, 1 H), 7.96 (s, 1 H),7.84 (d, 2 H), 7.76 (d, 2 H), 7.37 (d, 1 H), 4.77 (q, 2 H), 3.78-3.68 (m,1 H), 2.77 (d, 2 H), 2.16 (s, 3 H), 1.93 (t, 2 H), 1.76-1.74 (m, 2 H),1.59 (q, 2 H), 1.28 (t, 3 H) | 474 | Method 29 andMethod 45 |
| 69 | CF₂— | H | H | Me | Et | H | (400.132 MHz) 9.84 (s, 1 H), 8.56 (d, 1 H), 8.29 (q, 1 H), 7.89 (s, 1 H), | 373 | Method 2 and |
| | | | | | | | 7.82 (d, 2 H), 7.76 (d, 2 H), 7.28 (t, 1 H), 7.33 (d, 1 H), 4.77 (q, 2 H), | | Method 50 |
| | | | | | | | 2.78 (d, 3 H), 1.25 (t, 3 H) | | |
| 70 | CF₂— | F | H | Me | Et | H | (400.132 MHz) 10.02 (s, 1 H), 8.60 (d, 1 H), 8.01-7.99 (m, 1 H), 7.91 | 391 | Method 9 and |
| | | | | | | | (s, 1 H), 7.82 (d, 1 H), 7.66 (t, 1 H), 7.50 (dd, 1 H), 7.38 (d, 1 H), 7.30 | | Method 50 |
| | | | | | | | (t, 1 H), 4.78 (q, 2 H), 2.78 (d, 3 H), 1.27 (t, 3 H) | | |
| 71 | CF₂— | H | H | 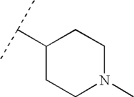 | Et | H | (400.132 MHz, CDCl3) 8.47 (d, 1 H), 7.77 (d, 2 H), 7.67 (d, 2 H),7.59 (s, 1 H), 7.23 (s, 1 H), 7.05 (d, 1 H), 6.80 (t, 1 H), 5.94 (d, 1 H),4.76 (q, 2 H), 4.05-3.97 (m, 1 H), 2.85 (d, 2 H), 2.33 (s, 3 H), 2.18 (t,2 H), 2.08-2.05 (m, 2 H), 1.61 (q, 2 H), 1.36 (t, 3 H) | 456 | Method 29 andMethod 50 |
| 72 | cPr- | H | H | Me | iPr | H | (400.132 MHz) 9.76 (s, 1 H), 8.44 (d, 1 H), 8.29 (q, 1 H), 7.82-7.77 | 377 | Method 2 and |
| | | | | | | | (m, 4 H), 7.11 (d, 1 H), 5.83 (septet, 1 H), 2.78 (d, 3 H), 2.22-2.15 (m, | | Method 54 |
| | | | | | | | 1 H), 1.59 (d, 6 H), 1.02-0.98 (m, 4 H) | | |
| 73 | cPr- | F | H | Me | iPr | H | (400.132 MHz) 9.95 (s, 1 H), 8.48 (d, 1 H), 7.98-7.96 (m, 1 H), 7.81 | 395 | Method 9 and |
| | | | | | | | (d, 1 H), 7.64 (t, 1 H), 7.52 (d, 1 H), 7.42 (s, 1 H), 7.16 (d, 1 H), 5.77 | | Method 54 |
| | | | | | | | (septet, 1 H), 2.78 (d, 3 H), 2.22-2.16 (m, 1 H), 1.60 (d, 6 H), 1.04-0.99 | | |
| | | | | | | | (m, 4 H) | | |
| 74 | cPr- | H | H | 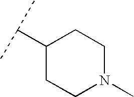 | iPr | H | (400.132 MHz, CDCl3) 8.37 (d, 1 H), 7.75 (d, 2 H), 7.70 (d, 2 H),7.33 (s, 1 H), 7.28 (s, 1 H), 6.95 (d, 1 H), 5.92 (d, 1 H), 5.72 (septet,1 H), 4.04-3.96 (m, 1 H), 2.87-2.80 (m, 2 H), 2.17 (t, 2 H), 2.07-2.01(m, 3 H), 1.77 (s, 3 H), 1.65 (d, 6 H), 1.59 (q, 2 H), 1.20-1.17 (m,2 H), 1.08-1.03 (m, 2 H) | 460 | Method 29 andMethod 54 |

### Example 75

### N-(1,1-Dioxidotetrahydro-3-thienyl)-4-{[4-(1-isopropyl-2-methyl-1H-imidazol-5-yl)pyrimidin-2-yl]amino}benzamide

HBTU (257 mg) was added to a stirred suspension of 4-{[4-(1-isopropyl-2-methyl-1H-imidazol-5-yl)pyrimidin-2-yl]amino}benzoic acid sodium salt (Method 56; 240 mg) in DMF (8 ml). The mixture was stirred at ambient temperature for 20 minutes, then 3-aminotetrahydrothiophene-S,S-dioxide hydrochloride (170 mg) and DIPEA (139 μl) was added. The mixture was stirred at ambient temperature for 18 hours, then diluted with EtOAc (80 ml), washed with 2N NaOH (80 ml). The aqueous layer was extracted with further EtOAc (80 ml) and the organics were concentrated in vacuo. The residue was purified by RPHPLC. Fractions containing product were poured onto a 10 g SCX-2 column, washed with MeOH, then eluted with methanolic ammonia. Evaporation of the basic eluent gave the title compound as a white solid (150 mg, 49%). NMR 9.73 (s, 1H), 8.52 (d, 1H), 8.44 (d, 1H), 7.85-7.77 (m, 4H), 7.45 (s, 1H), 7.11 (d, 1H), 5.71-5.64 (m, 1H), 4.73-4.63 (m, 1H), 3.53-3.37 (m, 2H), 3.25-3.02 (m, 2H), 2.50 (s, 3H), 2.47-2.37 (m, 1H), 2.28-2.14 (m, 1H), 1.47 (d, 6H); m/z 455.

### Examples 76-89

The following compounds were prepared by the procedure of Example 75 and on the same scale, using the appropriate amine starting material (method of preparation indicated if not commercially available) and the appropriate acid.

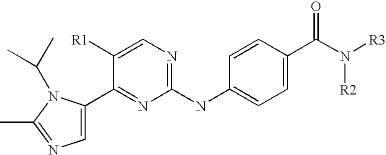

| Ex | R1 | R2 | R3 | NMR | m/z | Amine/acid |
|---|---|---|---|---|---|---|
| 76 | H | Me | 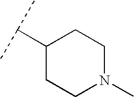 | (399.902 MHz) 9.22 (s, 1 H), 8.40 (d, 1 H), 7.72 (d, 2 H), 7.37 (s, 1 H), 7.29 (d,2 H), 7.03 (d, 1 H), 5.64-5.58 (m, 1 H), 3.91-3.82 (m, 1 H), 2.97 (s, 3 H),2.82-2.79 (m, 2 H), 2.49 (s, 3 H), 2.15 (s, 3 H), 1.89-1.81 (m, 4 H), 1.60-1.55(m, 2 H), 1.46 (d, 6 H) | 448 | Method 56 |
| 77 | H | Me | 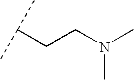 | (399.902 MHz) 9.22 (s, 1 H), 8.40 (d, 1 H), 7.72 (d, 2 H), 7.37 (s, 1 H), 7.32 (d,2 H), 7.03 (d, 1 H), 5.65-5.57 (m, 1 H), 3.49-3.43 (m, 2 H), 3.03-2.97 (m,2 H), 2.95 (s, 3 H), 2.49 (s, 3 H), 2.17 (s, 6 H), 1.47 (d, 6 H) | 422 | Method 56 |
| 78 | H | H | 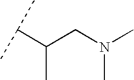 | (400.132 MHz) 9.75 (s, 1 H), 8.45 (d, 1 H), 7.97 (d, 1 H), 7.83-7.78 (m, 4 H),7.46 (s, 1 H), 7.12 (d, 1 H), 5.77-5.70 (m, 1 H), 4.19-4.12 (m, 1 H), 2.52 (s,6 H), 2.41-2.36 (m, 1 H), 2.25-2.20 (m, 1 H), 2.17 (s, 3 H), 1.49 (d, 6 H), 1.13(d, 3 H); | 422 | Method 56 |
| 79 | H | H | 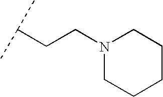 | (400.132 MHz) 9.77 (s, 1 H), 8.45 (d, 1 H), 8.24 (t, 1 H), 7.82-7.77 (m, 4 H),7.46 (s, 1 H), 7.13 (d, 1 H), 5.78-5.71 (m, 1 H), 3.37-3.33 (m, 2 H), 2.51 (s,3 H), 2.44-2.38 (m, 6 H), 1.52-1.47 (m, 10 H), 1.40-1.35 (m, 2 H); | 448 | Method 56 |
| 80 | H | H | 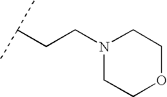 | (400.132 MHz) 9.77 (s, 1 H), 8.45 (d, 1 H), 8.27 (t, 1 H), 7.82-7.77 (m, 4 H),7.47 (s, 1 H), 7.13 (d, 1 H), 5.78-5.71 (m, 1 H), 3.58-3.56 (m, 4 H), 3.42-3.37(m, 2 H), 2.51 (s, 3 H), 2.48-2.40 (m, 6 H), 1.48 (d, 6 H); | 450 | Method 56 |
| 81 | H | H | 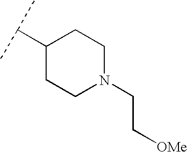 | (400.132 MHz) 9.74 (s, 1 H), 8.45 (d, 1 H), 8.05 (d, 1 H), 7.83-7.76 (m, 4 H),7.46 (s, 1 H), 7.12 (d, 1 H), 5.76-5.70 (m, 1 H), 3.77-3.70 (m, 1 H), 3.45-3.39(m, 4 H), 3.24 (s, 3 H), 2.91-2.85 (m, 2 H), 2.46 (s, 3 H), 2.07-1.99 (m, 2 H),1.78-1.72 (m, 2 H), 1.61-1.52 (m, 2 H), 1.49 (d, 6 H); | 478 | Step 2 ofExample 29 inWO03/022840and Method 56 |
| 82 | H | H | 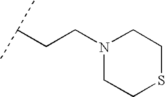 | (400.132 MHz) 9.77 (s, 1 H), 8.45 (d, 1 H), 8.24 (t, 1 H), 7.81-7.77 (m, 4 H),7.47 (s, 1 H), 7.13 (d, 1 H), 5.78-5.71 (m, 1 H), 3.39-3.34 (m, 2 H), 2.74-2.68(m, 6 H), 2.63-2.58 (m, 4 H), 2.51 (s, 3 H), 1.48 (d, 6 H); | 466 | Method 56 |
| 83 | H | H | 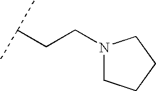 | (400.132 MHz) 9.77 (s, 1 H), 8.45 (d, 1 H), 8.29 (t, 1 H), 7.84-7.76 (m, 4 H),7.46 (s, 1 H), 7.12 (d, 1 H), 5.78-5.71 (m, 1 H), 3.42-3.33 (m, 2 H), 2.60-2.54(m, 6 H), 2.51 (s, 3 H), 1.71-1.65 (m, 4 H), 1.48 (d, 6 H); | 434 | Method 56 |
| 84 | F | H | 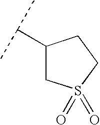 | 9.83 (s, 1 H), 8.60 (d, 1 H), 8.51 (d, 1 H), 7.81 (d, 2 H), 7.75 (d, 2 H), 7.38 (d,1 H), 5.49-5.40 (m, 1 H), 4.71-4.64 (m, 1 H), 3.52-3.45 (m, 1 H), 3.41-3.32(m, 1 H), 3.23-3.13 (m, 1 H), 3.09-3.02 (m, 1 H), 2.53 (s, 3 H), 2.44-2.38 (m,1 H), 2.27-2.17 (m, 1 H), 1.47 (d, 6 H) | 473 | Method 58 |
| 85 | F | Me | 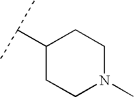 | 9.74 (s, 1 H), 8.57 (d, 1 H), 7.69 (d, 2 H), 7.36 (d, 1 H), 7.30 (d, 2 H), 5.48-5.38(m, 1 H), 2.80 (s, 3 H), 2.79-2.73 (m, 2 H), 2.52 (s, 3 H), 2.50-2.47 (m, 1 H),2.11 (s, 3 H), 1.84-1.70 (m, 4 H), 1.58-1.51 (m, 2 H), 1.44 (d, 6 H) | 466 | Method 58 |
| 86 | F | Me | 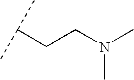 | 9.73 (s, 1 H), 8.57 (d, 1 H), 7.69 (d, 2 H), 7.36 (d, 1 H), 7.32 (d, 2 H), 5.48-5.38(m, 1 H), 3.45-3.36 (m, 2 H), 2.94 (s, 3 H), 2.52 (s, 3 H), 2.43-2.37 (m, 2 H),2.08 (s, 6 H), 1.44 (d, 6 H) | 440 | Method 58 |
| 87 | F | H | 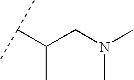 | 9.78 (s, 1 H), 8.59 (d, 1 H), 7.90 (d, 1 H), 7.79 (d, 2 H), 7.72 (d, 2 H), 7.37 (d,1 H), 5.50-5.41 (m, 1 H), 4.18-4.08 (m, 1 H), 2.53 (s, 3 H), 2.43-2.33 (m, 1 H),2.25-2.18 (m, 1 H), 2.16 (s, 6 H), 1.47 (d, 6 H), 1.12 (d, 3 H) | 440 | Method 58 |
| 88 | F | H | 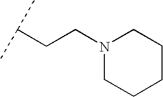 | 9.80 (s, 1 H), 8.58 (s, 1 H), 8.20-8.14 (m, 1 H), 7.77 (d, 2 H), 7.71 (d, 2 H), 7.38(d, 1 H), 5.50-5.42 (m, 1 H), 3.39-3.31 (m, 2 H), 2.52 (s, 3 H), 2.45-2.34 (m,6 H), 1.46 (d, 6 H), 1.42-1.30 (m, 6 H) | 466 | Method 58 |
| 89 | F | H | 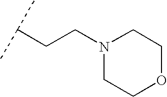 | 9.80 (s, 1 H), 8.58 (d, 1 H), 8.23-8.17 (m, 1 H), 7.78 (d, 2 H), 7.71 (d, 2 H), 7.37(d, 1 H), 5.50-5.40 (m, 1 H), 3.60-3.52 (m, 4 H), 3.40-3.33 (m, 2 H), 2.53 (s,3 H), 2.48-2.37 (m, 6 H), 1.46 (d, 6 H) | 468 | Method 58 |

### Example 90

### 4-[4-(3-Isopropyl-2-methyl-3H-imidazole-4-yl)-pyrimidin-2-ylamino]-benzamide

To 4-[4-(3-isopropyl-2-methyl-3H-imidazol-4-yl)pyrimidin-2-ylamino]-benzonitrile (Method 20; 165 mg, 0.52 mmol) was added EtOH (5.0 ml), water (2.5 ml) and KOH (54 mg, 0.1 mmol). The reaction was heated at reflux for 12 hours, the EtOH was removed in vacuo and the solution was extracted with DCM (3×50 ml), dried and the solvent removed to yield a white solid. DCM (3 ml) was added to the solid followed by ether. The solid was filtered and dried the give the title compound (94 mg, 54%). NMR (400.132 MHz) 9.72 (s, 1H), 8.45 (d, 1H), 7.84 (d, 2H), 7.79-7.77 (m, 3H), 7.46 (s, 1H), 7.15-7.12 (m, 2H), 5.74 (septet, 1H), 2.52 (s, 3H), 1.49 (d, 6H); m/z 336.

### Example 91

### 4-[5-Fluoro-4-(3-isopropyl-2-methyl-3H-imidazol-4-yl)-pyrimidin-2-ylamino]-benzamide

To 4-[5-fluoro-4-(3-isopropyl-2-methyl-3H-imidazol-4-yl)-pyrimidin-2-ylamino]-benzonitrile (Method 21; 180 mg, 0.54 mmol) was added EtOH (5.0 ml), water (2.5 ml) and KOH (54 mg, 0.1 mmol). The reaction was heated at reflux for 12 hours, the EtOH was removed in vacuo and the reaction was extracted with DCM (3×50 ml), dried and the solvent removed to yield a white solid. DCM (3 ml) was added to the solid followed by ether. The solid was filtered and dried (108 mg, 57%). NMR (400.132 MHz) 9.82 (s, 1H), 8.61 (d, 1H), 7.83 (d, 2H), 7.79 (brs, 1H), 7.73 (d, 2H), 7.39 (d, 1H), 7.14 (brs, 1H), 5.48 (septet, 1H), 2.54 (s, 3H), 1.47 (d, 6H); m/z 355.

### Example 92

### 2-Cyano-N-cyclopropyl-4-{[5-fluoro-4-(1-isopropyl-2-methyl-1H-imidazol-5-yl)pyrimidin-2-yl]amino}benzamide

5-Fluoro-4-(3-isopropyl-2-methyl-3H-imidazol-4-yl)-pyrimidin-2-ylamine (Method 17, 0.20 g, 0.85 mmol), PdOAc₂ (16 mg, 0.068 mmol), XANTPHOS (60 mg, 0.10 mmol), caesium carbonate (0.42 g, 1.3 mmol) and 4-chloro-2-cyano-N-cyclopropyl-benzamide (Method 24, 0.24 g, 1.10 mmol) were added to dioxane (7 ml) under a inert atmosphere and heated at 150° C. in a microwave for 1 hour. Purification on silica using 0-10% MeOH in DCM as eluent gave a yellow foam, further purification by RPHPLC gave the title compound as a colourless foam (187 mg, 53%). NMR (399.902 MHz, DMSO-d₆+AcOH-d₄, 373K) 11.48 (brs, 1H), 8.52 (s, 1H), 8.23 (s, 1H), 7.90 (d, 1H), 7.63 (d, 1H), 7.41 (s, 1H), 5.35 (septet, 1H), 2.68-2.62 (m, 1H), 2.52 (s, 3H), 1.45 (d, 6H), 0.99-0.93 (m, 2H), 0.91-0.87 (m, 2H); m/z 420.

### Example 93

### 2-Cyano-N-cyclopropyl-4-{[4-(1-isopropyl-2-methyl-1H-imidazol-5-yl)pyrimidin-2-yl]amino}benzamide

The title compound was prepared in a similar manner to Example 92 except using 4-(3-isopropyl-2-methyl-3H-imidazol-4-yl)-pyrimidin-2-ylamine (Method 18) in place of Method 17. NMR (399.902 MHz, DMSO-d₆+AcOH-d₄, 373K) 8.44 (d, 1H), 8.27 (s, 1H), 7.95 (d, 1H), 7.62 (d, 1H), 7.41 (s, 1H), 7.08 (d, 1H), 5.52 (septet, 1H), 2.68-2.62 (m, 1H), 2.49 (s, 3H), 1.45 (d, 6H), 0.99-0.93 (m, 2H), 0.91-0.87 (m, 2H); m/z 402.

### Example 94

### 4-{[5-Fluoro-4-(1-isopropyl-2-methyl-1H-imidazol-5-yl)pyrimidin-2-yl]amino}-N-(1-methylpiperidin-4-yl)benzamide

5-Fluoro-4-(3-isopropyl-2-methyl-3H-imidazol-4-yl)-pyrimidin-2-ylamine (Method 17, 149 mg, 0.63 mmol), 4-iodo-N-(1-methylpiperidin-4-yl)benzamide (Method 29, 239 mg, 0.69 mmol), palladium acetate (9 mg, 0.04 mmol), XANTPHOS (33 mg, 0.057 mmol) and caesium carbonate (412 mg, 1.26 mmol) were stirred at reflux in 1,4-dioxane (7 ml) under an inert atmosphere for one hour. The reaction mixture was cooled, filtered, the filtrate was evaporated in vacuo and the residue purified by reverse phase HPLC. Trituration with ether afforded the title compound as a colourless solid (190 mg, 67%). NMR 9.78 (s, 1H), 8.58 (d, 1H), 7.99 (d, 1H), 7.79 (d, 2H), 7.71 (d, 2H), 7.37 (d, 1H), 7.50-7.39 (m, 1H), 3.78-3.62 (m, 1H), 2.76 (d, 2H), 2.52 (s, 3H), 2.15 (s, 3H), 1.98 (t, 2H), 1.81-1.67 (m, 2H), 1.58 (apparent t, 3H), 1.46 (d, 6H); m/z 452.

### Example 95

### 4-{[5-Fluoro-4-(1-isopropyl-2-methyl-1H-imidazol-5-yl)pyrimidin-2-yl]amino}-N-(tetrahydro-2H-pyran-4-yl)benzamide

The title compound was prepared using the procedure and scale described above for Example 94 but utilizing 4-iodo-N-(tetrahydro-2H-pyran-4-yl)benzamide (Method 30) in place of 4-iodo-N-(1-methylpiperidin-4-yl)benzamide (Method 29). A colourless solid was obtained (160 mg, 58%). NMR 9.79 (s, 1H), 8.59 (d, 1H), 8.07 (d, 1H), 7.80 (d, 2H), 7.72 (d, 2H), 7.37 (d, 1H), 5.51-5.38 (m, 1H), 4.05-3.91 (m, 1H), 3.87 (d, 2H), 3.37 (app t, 2H), 2.53 (s, 3H), 1.74 (d, 2H), 1.65-1.49 (m, 2H), 1.46 (d, 6H); m/z 439.

### Example 96

### 4-{[5-Fluoro-4-(1-isopropyl-2-methyl-1H-imidazol-5-yl)pyrimidin-2-yl]amino}-N-piperidin-3-ylbenzamide

To a stirred solution of tert-butyl 3-[(4-{[5-fluoro-4-(1-isopropyl-2-methyl-1H-imidazol-5-yl)pyrimidin-2-yl]amino}benzoyl)amino]piperidine-1-carboxylate (Example 97; 50 mg, 0.093 mmol) in DCM (1 ml) was added trifluoroacetic acid (0.2 ml). The mixture was stirred for 2 hours, then the solvent remove in vacuo and the residue purified by ion exchange chromatography (SCX-2, 1 g), to afford the title compound as a white solid (29 mg, 71%).

NMR 9.78 (s, 1H), 8.58 (s, 1H), 7.88 (d, 1H), 7.79 (d, 2H), 7.71 (d, 2H), 7.37 (d, 1H), 5.32-5.35 (m, 1H), 3.88-3.70 (m, 1H), 3.00-2.66 (m, 2H), 2.52 (s, 3H under DMSO), 2.45-2.30 (m, 4H under DMSO), 1.90-1.56 (m, 2H), 1.46 (d, 6H); m/z 438.

### Example 97

### tert-Butyl 3-[(4-{[5-fluoro-4-(1-isopropyl-2-methyl-1H-imidazol-5-yl)pyrimidin-2-yl]amino}benzoyl)amino]piperidine-1-carboxylate

5-Fluoro-4-(3-isopropyl-2-methyl-3H-imidazol-4-yl)-pyrimidin-2-ylamine (Method 17; 149 mg, 0.63 mmol), tert-butyl 3-[(4-iodobenzoyl)amino]piperidine-1-carboxylate (Method 61; 297 mg, 0.69 mmol), palladium acetate (9 mg, 0.04 mmol), XANTPHOS (33 mg, 0.057 mmol) and caesium carbonate (412 mg, 1.26 mmol) were stirred at reflux in 1,4-dioxane (7 ml) under an inert atmosphere for one hour. The reaction mixture was cooled and filtered. The filtrate was evaporated in vacuo and the residue purified by reverse phase HPLC. Trituration with ether afforded the title compound as a white solid (232 mg, 69%). NMR 9.79 (s, 1H), 8.59 (d, 1H), 8.01 (d, 1H), 7.80 (d, 2H), 7.72 (d, 2H), 7.37 (d, 1H), 5.53-5.36 (m, 1H), 4.04-3.64 (m, 3H), 2.86-2.69 (m, 2H), 2.53 (s, 3H under DMSO) 2.54-2.35 (m, 2H under DMSO), 1.94-1.63 (m, 2H), 1.46 (d, 6H), 1.37 (s, 9H).

### Preparation of Starting Materials

### Method 1

### 4-Bromo-N,N-dimethyl-benzamide

4-Bromo benzoyl chloride (5.0 g, 22.8 mmol) was added to DCM (100 ml), and to this was added TEA (7.0 ml, 50.2 mmol) followed by the slow addition of dimethylamine (20 ml, 2.0N in THF). The reaction was stirred for 1 hour before being quenched with HCl (2.0 N; 50 ml), the reaction was extracted with DCM (2×100 ml), dried and the solvent was removed in vacuo to yield a white solid (5.1 g, 98%). NMR (299.954 MHz, CDCl₃) 7.57 (d, 2H), 7.30 (d, 2H), 3.10 (s, 3H), 2.98 (s, 3H); m/z 228

### Method 2

### 4-Bromo-N-methyl-benzamide

4-Bromo benzoyl chloride (5.0 g, 22.8 mmol) was added to DCM (100 ml), to this was added TEA (7.0 ml, 50.2 mmol) followed by the slow addition of methylamine (20 ml, 2.0N in THF). The reaction was stirred for 1 hour before being quenched with HCl (2.0 N; 50 ml), the reaction was extracted with DCM (2×100 ml), dried and the solvent was removed in vacuo to yield a white solid (4.8 g, 98%). NMR (299.954 MHz, CDCl₃) 7.62 (d, 2H), 7.55 (d, 2H), 6.16 (s, 3H), 3.00 (d, 6H); m/z 215

### Method 3

### 4-Bromo-N-cyclopropyl-benzamide

4-Bromo benzoyl chloride (5.0 g, 22.8 mmol) was added to DCM (100 ml), to this was added TEA (7.0 ml, 50.2 mmol) followed by the slow addition of cyclopropyl amine (1.7 g, 29.6 mmol). The reaction was stirred for 1 hour before being quenched with 2.0 N HCl (50 ml), the reaction was extracted with DCM (2×100 ml), dried and the solvent was removed in vacuo to yield a white solid (4.7 g, 86%). NMR (299.954 MHz, CDCl₃) 7.60 (d, 2H), 7.54 (d, 2H), 6.27 (s, 1H), 2.93-2.85 (m, 1H), 0.87 (q, 2H), 0.64-0.59 (m, 2H); m/z 241.

### Method 4

### 4-Bromo-2-fluorobenzamide

4-Bromo-2-fluoro-cyanobenzne (2.0 g, 10 mmol) and sodium perborate (3.0 g, 20 mmol) were dissolved in dioxane (40 ml), water (40 ml) and heated at reflux for 1 hour. An extra 2.0 g of sodium perborate was added and the reaction was refluxed for 1 hour. The reaction was cooled, extracted with DCM (2×100 ml), dried and the solvent was removed in vacuo to yield a white solid. Ether was added to dissolve any remaining starting material and the solid was stirred for 10 minutes and filtered to give the title compound (1.7 g, 88%). NMR (400.132 MHz) 7.74 (brs, 1H), 7.68 (brs, 1H), 7.64 (d, 1H), 7.61 (t, 1H), 7.50 (d, 1H).

### Method 5

### 4-Bromo-2-chlorobenzamide

4-Bromo-2-chloro-cyanobenzne (2.0 g, 9.3 mmol) and sodium perborate (4.3 g, 28 mmol) were dissolved in dioxane (40 ml), water (40 ml) and heated at reflux for 1 hour. An extra 2.0 g of perborate was added and the reaction was refluxed for 1 hour. The reaction was cooled, extracted with DCM (2×100 ml), dried and the solvent was removed in vacuo to yield a white solid. Ether was added to dissolve any remaining starting material and the solid was stirred for 10 minutes and filtered (1.35 g, 62%). NMR (299.954 MHz, CDCl₃) 12.61 (s, 1H), 12.50 (s, 1H), 12.38-12.31 (m, 2H), 12.13 (d, 1H).

### Method 6

### 4-Bromo-2-methyl-N,N-dimethyl-benzamide

4-Bromo-2-methylbenzoic acid (1.0 g, 4.65 mmol) was added to DCM (50 ml), to this was added oxalyl chloride (0.61 ml, 6.97 mmol) and 4 drops of DMF, the reaction was stirred until no gas was liberated (approx 30 mins). To the reaction was added dimethylamine (20 ml, 2N in THF) and the reaction was stirred for a further 10 minutes before being quenched with saturated sodium bicarbonate (50 ml) and extracted with ether (2×100 ml), dried and the solvent was removed in vacuo to yield a yellow gum. The gum was purified via column chromatography eluting with 40% ether/isohexane, 60% ether/isohexane and finally ether. A clear gum obtained (1.0 g 89%). NMR (299.954 MHz, CDCl₃) 7.38 (s, 1H), 7.35 (d, 1H), 7.04 (d, 1H), 3.12 (s, 3H), 2.83 (s, 3H), 2.27 (s, 3H); m/z 243.

### Method 7

### 4-Bromo-2-methyl-N-methyl-benzamide

4-Bromo-2-methylbenzoic acid (1.0 g, 4.65 mmol) was added to DCM (50 ml), to this was added oxalyl chloride (0.61 ml, 6.97 mmol) and 4 drops of DMF, the reaction was stirred until no gas was liberated (approx 30 mins). To the reaction was added methylamine (30 ml, 2N in THF) and the reaction was stirred for a further 10 minutes before being quenched with saturated sodium bicarbonate (60 ml), extracted with ether (2×100 ml), dried and the solvent was removed in vacuo to yield a white solid. The solid was dissolved in a minimum amount of DCM, to this was added ether and isohexane until a white solid precipitated. The solid was filtered and dried (11.0 g, 94%). NMR (299.954 MHz, CDCl₃) 7.37 (s, 1H), 7.32 (d, 1H), 7.20 (d, 1H), 5.76 (brs, 1H), 2.98 (d, 3H), 2.41 (s, 3H); m/z 229.

### Method 8

### 4-Bromo-2-fluoro-N,N-dimethyl-benzamide

4-Bromo-2-fluorobenzoic acid (2.0 g, 9.1 mmol) was added to DCM, to this was added oxalyl chloride (1.2 ml, 13.7 mmol) and 4 drops of DMF, the reaction was stirred until no gas was liberated (approx 30 mins). To the reaction was added triethylamine (6.3 ml, 48 mmol) followed by dimethylamine (10 ml). The reaction was stirred for 10 minutes before being quenched with HCl (2.0 N, 50 ml), extracted with DCM (2×100 ml), dried and the solvent was removed in vacuo. The obtained gum was purified via column chromatography eluting with 20% ether/isohexane, 40% ether/isohexane and finally ether to yield a clear gum (1.2 g, 54%). NMR (299.954 MHz, CDCl₃) 7.36 (d, 1H), 7.31-1.24 (m, 2H), 3.12 (s, 3H), 2.92 (s, 3H); m/z 247.

### Method 9

### 4-Bromo-2-fluoro-N-methyl-benzamide

4-Bromo-2-fluorobenzoic acid (2.0 g, 9.1 mmol) was added to DCM, to this was added oxalyl chloride (1.2 ml, 13.7 mmol) and 4 drops of DMF, the reaction was stirred until no gas was liberated (approx 30 mins). To the reaction was added triethylamine (6.3 ml, 46.0 mmol) followed by methylamine (10 ml, 2.0N in THF). The reaction was stirred for 10 mins before being quenched with HCl (2.0 N, 50 ml), extracted with DCM (2×100 ml), dried and the solvent was removed in vacuo. The obtained solid was purified by passing through a plug of silica eluting with DCM, the obtained yellow solid was added to 20% ether/isohexane and stirred for 10 minutes before being filtered and dried. A white solid was obtained (1.1 g, 52%).

NMR (299.954 MHz, CDCl₃) 8.00 (t, 1H), 7.41 (d, 1H), 7.31 (d, 1H), 6.64 (brs, 1H), 3.03 (d, 3H); m/z 232.

### Method 10

### 4-Bromo-2-fluoro-N-cyclopropyl-benzamide

4-Bromo-2-fluorobenzoic acid (2.0 g, 9.1 mmol) was added to DCM, to this was added oxalyl chloride (1.2 ml, 13.7 mmol) and 4 drops of DMF, the reaction was stirred until no gas was liberated (approx 30 mins). To the reaction was added triethylamine (6.3 ml, 46.0 mmol) followed by cyclopropylamine (1.06 g, 18 mmol). The reaction was stirred for 10 minutes before being quenched with HCl (2.0 N, 50 ml), extracted with DCM (2×100 ml), dried and the solvent was removed in vacuo to yield a solid. The solid was stirred in ether (30 ml) for 10 minutes before being filtered and dried. A white solid was obtained (1.4 g, 60%). NMR (299.954 MHz, CDCl₃) 7.99 (t, 1H), 7.40 (d, 1H), 7.29 (d, 1H), 6.69 (brs, 1H), 2.97-2.89 (m, 1H), 0.88 (q, 2H), 0.65-0.60 (m, 2H); m/z 259.

### Method 11

### 4-Bromo-2-chloro-N,N-dimethyl-benzamide

4-Bromo-2-chlorobenzoic acid (1.0 g, 4.3 mmol) was added to DCM, to this was added oxalyl chloride (0.5 ml, 5.5 mmol) and 4 drops of DMF. The reaction was stirred until no gas was liberated (approx 30 mins). To the reaction was added triethylamine (2.9 ml, 21.3 mmol) followed by dimethylamine (10 ml). The reaction was stirred for 10 minutes before being quenched with HCl (2.0 N, 50 ml), extracted with DCM (2×100 ml), dried and the solvent was removed in vacuo to yield a gum. The gum was purified via column chromatography eluting with 20% ether/isohexane, 40% ether/isohexane and finally ether. A clear gum was obtained (1.05 g, 95%). NMR (299.954 MHz, CDCl₃) 7.58 (d, 1H), 7.46 (dd, 1H), 7.17 (d, 1H), 3.12 (s, 3H), 2.86 (s, 3H); m/z 263.

### Method 12

### 4-Bromo-2-chloro-N-cyclopropyl-benzamide

4-Bromo-2-chlorobenzoic acid (1.0 g, 4.3 mmol) was added to DCM, to this was added oxalyl chloride (0.5 ml, 5.5 mmol) and 4 drops of DMF. The reaction was stirred until no gas was liberated (approx 30 minutes). To the reaction was added triethylamine (2.9 ml, 21.3 mmol) followed by cyclopropylamine (0.48 g, 8.5 mmol). The reaction was stirred for 10 minutes before being quenched with HCl (2.0 N, 50 ml), extracted with DCM (2×100 ml), dried and the solvent was removed in vacuo to yield a solid. Ether was added to the solid, this was stirred for 10 minutes before being filtered and dried. A white solid was obtained (1.0 g, 86%). M/z 275.

### Method 13

### 4-Bromo-2-methylbenzamide

4-Bromo-2-methylcyanobenzene (10 g, 51 mmol) was, added to EtOH/water (4:1, 180 ml), to this was added KOH (6.3 g, 112 mmol) and the reaction was heated at reflux for 6 hours. The reaction was allowed to cool (solid precipitated). The EtOH was removed in vacuo until 25% of the original volume remained. The solid was filtered and dried. NMR (299.955 MHz) 7.71 (s, 1H), 7.45 (s, 1H), 7.40-7.38 (m, 2H), 7.28 (d, 1H), 2.34 (s, 3H); m/z 215.

### Method 14

### 4-Bromo-2-fluoro-N-(2-hydroxy-ethyl)-benzamide

4-Bromo-2-fluorobenzoic acid (2.0 g, 9.2 mmol), HATU (4.1 g, 11.0 mmol) and DIPEA (2.4 ml, 13.7 mmol) were pre-mixed in DCM (70 ml) and stirred for 10 minutes. To this was added 2-hydroxyethylamine (0.83 g, 13.7 mmol) and the reaction was stirred for a further 1 hour before being quenched with water (50 ml). The reaction was extracted with DCM (2×100 ml), dried and the solvent was removed in vacuo to yield a yellow gum. The gum was purified via column chromatography eluting with 20%, EtOAc/isohexane, 40% EtOAc/isohexane and finally EtOAc. A waxy solid was obtained (2.15 g, 90%). NMR (299.954 MHz, CDCl₃) 7.97 (t, 1H), 7.41 (d, 1H), 7.32 (d, 1H), 7.07 (s, 1H), 3.84 (q, 2H), 3.65 (q, 2H), 2.44 (t, 1H); m/z 264

### Method 15

### 4-Bromo-N-(2-hydroxy-ethyl)-benzamide

4-Bromobenzoic acid (5.0 g, 24.9 mmol), DMTMM (8.8 g, 30 mmol) and DIPEA (6.1 ml, 38 mmol) were pre-mixed in DCM (70 ml) and stirred for 10 minutes, to this was added 2-hydroxyethylamine (1.82 g, 30 mmol) and the reaction was stirred for a further 1 hour before being quenched with 2.0 N HCl (50 ml). The reaction was extracted with DCM (2×100 ml), dried and the solvent was removed in vacuo to yield a yellow solid. The solid was dissolved in hot DCM (20 ml), the solution was allowed to cool before the addition of ether (20 ml). White solid precipitated, this was filtered and dried. NMR (299.955 MHz) 8.44 (s, 1H), 7.78 (d, 2H), 7.62 (d, 2H), 4.69 (t, 1H), 3.49 (t, 2H), 3.29 (t, 2H); m/z 245.

### Method 16

### (2Z)-3-(Dimethylamino)-2-fluoro-1-(1-isopropyl-2-methyl-1H-imidazol-5-yl)prop)-2-en-1-one

To a stirred solution of (2E)-3-(dimethylamino)-1-(1-isopropyl-2-methyl-1H-imidazol-5-yl)prop-2-en-1-one, (Method 24 of WO 03/076436; 5.53 g, 25 mmol) in MeOH (100 ml) at ambient temperature was added in portions over ˜5 mins (1-chloromethyl-4-fluoro-1,4-diazoniabicyclo[2.2.2]octane bis(tetrafluoroborate) (14.16 g, 40 mmol). The temperature was maintained at 25-30° C. by slight cooling. After stirring for 90 mins the reaction mixture was cooled in ice/acetone and filtered. The filtrate was evaporated under reduced pressure and the residue was taken into DCM. It was washed with aq. ammonia, brine, dried (Na₂SO₄) and evaporated under reduced pressure. The title compound was isolated by MPLC on silica gel using two separate columns (10% EtOH/EtOAc, then 3.5% EtOH/DCM) as a golden viscose oil, which crystallized on standing over several weeks. Yield=2.50 g (42%). NMR 1.40 (d, 6H), 2.38 (s, 3H), 3.05 (s, 6H), 4.70 (septet, 1H), 6.96 (d, 1H), 7.08 (s, 1H); fluorine

NMR (376 MHz): −166.7 (d); m/z 240.

### Method 17

### 5-Fluoro-4-(3-isopropyl-2-methyl-3H-imidazol-4-yl)-pyrimidin-2-ylamine

(2Z)-3-(Dimethylamino)-2-fluoro-1-(1-isopropyl-2-methyl-1H-imidazol-5-yl)prop-2-en-1-one (Method 16; 4.0 g, 16.7 mmol) and guanidine carbonate (6.6 g, 37 mmol) were pre-mixed in butanol (80 ml) and heated at reflux for 30 hours. The reaction was allowed to cool before being quenched with water (200 ml) the reaction was then extracted with DCM (2×200 ml), dried and solvent was removed in vacuo to yield a yellow solid. The solid was dissolved in minimum amount of warm DCM, this was then allowed to cool before the addition of ether. An off white solid precipitated this was filtered and dried. The process was repeated to obtain second crop of product (3.18 g, 81%). NMR (299.954 MHz, CDCl₃) 8.15 (d, 1H), 7.54 (d, 1H), 7.26 (s, 1H), 5.40 (septet, 1H), 4.88 (s, 2H), 2.59 (s, 3H), 1.56 (d, 6H); m/z 236

### Method 18

### 4-(3-Isopropyl-2-methyl-3H-imidazol-4-yl)-pyrimidin-2-ylamine

(2E)-3-(Dimethylamino)-1-(1-isopropyl-2-methyl-1H-imidazol-5-yl)prop-2-en-1-one, (Method 24 of WO 03/076436 4.0 g, 18 mmol) and guanidine carbonate (7.2 g, 40 mmol) were pre-mixed in 2-methoxyethanol (80 ml) and heated at reflux for 30 hours. The reaction was allowed to cool before being quenched with water (50 ml). The reaction was then extracted with DCM (2×200 ml), dried and solvent was removed in vacuo to yield a yellow solid. The solid was dissolved in minimum amount of warm DCM, this was then allowed to cool before the addition of ether. An off white solid precipitated this was filtered and dried. The process was repeated to obtain second crop of product (3.18 g, 81%). NMR (299.954 MHz, CDCl₃) 8.22 (d, 1H), 7.33 (s, 1H), 6.80 (d, 1H), 5.45 (septet, 1H), 5.10 (s, 2H), 2.56 (s, 3H), 1.54 (d, 6H); m/z 218.

### Method 19

### 4-Bromo-2-chloro-N-methyl-benzamide

Sodium hydride (0.24 g, 5.0 mmol) was added to 4-bromo-2-chlorobenzamide (Method 5; 0.9 g, 3.85 mmol) in THF (20 ml). The reaction was stirred for 10 minutes before the addition of methyl iodide (0.36 ml, 5.8 mmol), the reaction was then stirred overnight. The reaction was quenched with saturated NH4Cl (20 ml), extracted with DCM (2×100 ml), dried and the solvent was removed in vacuo to yield a gum. The gum was chromatographed using DCM, 1% MeOH/DCM and finally 2.5% MeOH/DCM, to yield a mixture of mono and dialkylated products. Ether/isohexane (1:1) were added to the mixture, the dialkyl material dissolved to leave the required adduct, this was filtered and dried (0.12 g, 13%).

### Method 20

### 4-[4-(3-Isopropyl-2-methyl-3H-imidazol-4-yl)-pyrimidin-2-ylamino]-benzonitrile

The title compound was prepared using the procedure and scale described above for Example 1 but utilizing 4-bromo-cyanobenezene in place of 4-bromo-3-methyl-N,N-dimethyl-benzamide. The product was obtained as a white foam (165 mg, 75%). NMR (400.132 MHz, CDCl₃) 8.41 (d, 1H), 7.76 (d, 2H), 7.60 (d, 2H), 7.40 (s, 1H), 7.29 (s, 1H), 7.02 (d, 1H), 5.58 (septet, 1H), 2.6.1 (s, 3H), 1.55 (d, 6H); m/z 319.

### Method 21

### 4-[5-Fluoro-4-(3-isopropyl-2-methyl-3H-imidazol-4-yl)-pyrimidin-2-ylamino]-benzonitrile

The title compound was prepared using the procedure and scale described above for Example 1 but utilizing 4-bromo-cyanobenezene in place of 4-bromo-3-methyl-N,N-dimethyl-benzamide. A white foam was obtained (190 mg, 88%). NMR (400.132 MHz) 8.34 (s, 1H), 7.72 (d, 2H), 7.65-7.58 (m, 3H), 7.22 (s, 1H), 5.52 (septet, 1H), 2.62 (s, 3H), 1.57 (d, 6H); m/z 336.

### Method 22

### 4-Bromo-N-cyclopropyl-2-methyl-benzamide

Bromo-methylbenzoic acid (10 g, 46.5 mmol), and HBTU (23 g, 60.5 mmol) were dissolved in DMF (150 ml), cyclopropylamine (3.5 g, 60.5 mmol) was added, followed by DIPEA (21 ml, 121 mmol). The reaction was stirred overnight before the removal of the DMF in vacuo, the obtained gum was quenched with 2.0N NaOH (100 ml), the precipitated solid was filtered, dissolved in DCM, dried and solvent removed in vacuo to afford a off white solid (10.2 g, 87%). NMR (CDCl₃) 7.35 (s, 1H), 7.30 (d, 1H), 7.15 (d, 1H), 6.03 (brs, 1H), 2.91-2.82 (m, 1H), 2.39 (s, 3H), 0.90-0.83 (m, 2H), 0.63-0.57 (m, 2H); m/z 254.

### Method 23

### 4-Chloro-N-cyclopropyl-2-iodo-benzamide

2-Iodo-4-chlorobenzoic acid (10 g, 35.5 mmol) and HBTU (17.5 g, 46 mmol) were added to DMF (100 ml), followed by cyclopropylamine (2.6 g, 46 mmol) and DIPEA (17.5 ml, 92 mmol). The reaction was stirred overnight before being quenched with 2.0 NaOH (100 ml), extracted with DCM (3×200 ml), dried and solvent removed in vacuo to yield a dark yellow solid. This was passed through a pad of silica, eluting with DCM, the filtrate was concentrated in vacuo to yield a yellow solid. Ether (200 ml) was added, the slurry was sonicated for 20 mins, iso-hexane (100 ml) was then added and the system was stirred for 10 mins, filtered and dried to give a colourless solid (9.3 g, 82%). NMR (CDCl₃) 7.82 (s, 1H), 7.34 (d, 1H), 7.28 (d, 1H), 5.99 (s, 1H), 2.94-2.84 (m, 1H), 0.91-0.84 (m, 2H), 0.71-0.66 (m, 2H); m/z 322.

### Method 24

### 4-Chloro-2-cyano-N-cyclopropyl-benzamide

4-Chloro-N-cyclopropyl-2-iodo-benzamide (Method 23; 8.0 g, 25 mmol), copper (I) cyanide (9.0 g, 100 mmol), Pd₂(dba)₃ (0.9 g, 1 mmol), DPPF (1.7 g, 3 mmol) and tetraethylammonium cyanide (3.9 g, 25 mmol) were added to dioxane (80 ml) and heated at reflux for 2 hours. The reaction was filtered and the filtrate was removed in vacuo to yield a black solid. This was treated with water (200 ml), extracted with DCM (2×200 ml), dried and solvent removed in vacuo to yield a brown solid. Purification on silica using 0-2.5% MeOH in DCM as eluent gave the title compound as a brown solid. The brown solid was added to MeOH (50 ml), heated and then sonicated. The solid obtained was filtered and dried (4.4 g, 80%). NMR (CDCl₃) 8.77 (brs, 1H), 7.88 (s, 1H), 7.74 (d, 1H), 7.59 (d, 1H), 2.66-2.56 (m, 1H), 1.16-1.10 (m, 2H), 0.97-0.92 (m, 2H); m/z 221.

### Method 25

### (2Z)-3-(Dimethylamino)-2-fluoro-1-(1-ethyl-2-methyl-1H-imidazol-5-yl)prop-2-en-1-one

The title compound was prepared in a similar manner to Method 16 by using (2E)-3-(dimethylamino)-1-(1-ethyl-2-methyl-1H-imidazol-5-yl)prop-2-en-1-one (Method 23 in WO 03/076436) in place of (2E)-3-(dimethylamino)-1-(1-isopropyl-2-methyl-1H-imidazol-5-yl)prop-2-en-1-one. NMR 1.2 (t, 3H), 2.38 (s, 3H), 3.05 (s, 6H), 4.18 (q, 2H), 6.96 (d, 1H), 7.34 (s, 1H); Fluorine NMR (376 MHz)-168.2 (d); m/z 226.

### Method 26

### 5-Fluoro-4-(3-ethyl-2-methyl-3H-imidazol-4-yl)-pyrimidin-2-ylamine

The title compound was prepared in a similar manner to Method 17 by using (2Z)-3-(dimethylamino)-2-fluoro-1-(1-ethyl-2-methyl-1H-imidazol-5-yl)prop-2-en-1-one (Method 25) in place of (2Z)-3-(dimethylamino)-2-fluoro-1-(1-isopropyl-2-methyl-1H-imidazol-5-yl)prop-2-en-1-one. NMR 8.24 (d, 1H), 7.45 (d, 1H), 6.53 (br. s, 2H), 4.50 (q, 2H), 2.40 (s, 3H), 1.24 (t, 3H); m/z 222.

### Method 27

### (2Z)-3-(Dimethylamino)-2-fluoro-1-(1-cyclobutyl-2-methyl-1H-imidazol-5-yl)prop-2-en-1-one

The title compound was prepared in a similar manner to Method 16 by using (2E)-3-(dimethylamino)-1-(1-cyclobutyl-2-methyl-1H-imidazol-5-yl)prop-2-en-1-one (Method 37 in WO 03/076435) in place of (2E)-3-(dimethylamino)-1-(1-isopropyl-2-methyl-1H-imidazol-5-yl)prop-2-en-1-one. NMR (CDCl₃) 7.27-7.17 (m, 1H), 6.85 (d, 1H), 5.06-4.91 (m, 1H), 3.12-3.05 (m, 6H), 2.54-2.39 (m, 7H), 1.74 (m, 2H); m/z 252.

### Method 28

### 5-Fluoro-4-(3-cyclobutyl-2-methyl-3H-imidazol-4-yl)-pyrimidin-2-ylamine

The title compound was prepared in a similar manner to Method 17 by using (2Z)-3-(dimethylamino)-2-fluoro-1-(1-cyclobutyl-2-methyl-1H-imidazol-5-yl)prop-2-en-1-one (Method 27) in place of (2Z)-3-(dimethylamino)-2-fluoro-1-(1-isopropyl-2-methyl-1H-imidazol-5-yl)prop-2-en-1-one. NMR (CDCl₃) 8.26 (d, 1H), 7.21 (d, 1H), 6.58 (br. s, 1H), 5.17 (quintet, 1H), 3.45 (s, 3H), 2.42-2.29 (m, 4H), 1.80-1.64 (m, 2H); m/z 248.

### Method 29

### 4-Iodo-N-(1-methylpiperidin-4-yl)benzamide

1-Methylpiperidin-4-amine (5.0 g, 43.8 mmol) and triethylamine (7.3 ml, 52.5 mmol) were stirred in THF (200 ml) under an inert atmosphere. 4-Iodobenzoyl chloride (11.7 g, 43.8 mmol) was added in portions over 5 mins. Stirring was continued for a further 16 hours, then the solvent was evaporated in vacuo and the residue partitioned between EtOAc (200 ml) and 1M NaOH (100 ml). The organics were washed with water (100 ml) and brine (100 ml), dried and evaporated to afford the title compound as a colourless solid (13.2 g, 88%). NMR 8.26 (d, 1H), 7.82 (d, 2H), 7.61 (d, 2H), 3.77-6.62 (m, 1H), 2.74 (d, 2H), 2.14 (s, 3H), 1.92 (t, 2H), 1.97-1.85 (m, 2H), 1.55 (ap. q, 2H); m/z 345.

### Method 30

### 4-Iodo-N-(tetrahydro-2H-pyran-4-yl)benzamide

Tetrahydro-2H-pyran-4-amine (5.0 g, 49.4 mmol) and triethylamine (8.3 ml, 59.3 mmol) were stirred in THF (200 ml) under an inert atmosphere. 4-Iodobenzoyl chloride (13.2 g, 49.4 mmol) was added in portions over 5 mins. Stirring was continued for a further 16 hours, then the solvent was removed in vacuo. The resulting solid was sonicated in 1M NaOH solution (100 ml) for 10 mins then isolated by filtration and washed with fresh water (3×100 ml). The solid obtained was dried in vacuo at 60° C. for 24 hours (10.3 g, 57%). NMR 8.32 (d, 1H), 7.83 (d, 2H), 7.62 (d, 2H), 4.05-3.90 (m, 1H), 3.86 (d, 2H), 3.36 (app t, 2H), 1.73 (d, 2H), 1.64-1.46 (m, 2H); m/z 332.

### Method 31

### N-Ethyl-N-(5-methyl-isoxazol-4-yl)-isobutyramide

Ethyl-(5-methyl-isoxazol-4-yl)-amine hydrochloride (15 g, 0.092 mol) was added to DCM (200 ml), TEA (32 ml, 0.23 mol) was added, followed by the slow addition of iso-butryl chloride (10.7 g, 0.10 mol). The reaction was stirred for 30 minutes before the removal of the solvent in vacuo. The residue was treated with water (150 ml), extracted with ether (3×150 ml), dried and solvent removed in vacuo to yield a yellow oil (12.9 g, 72%). NMR (CDCl₃) 8.14 (s, 1H), 3.61 (q, 2H), 2.46-2.37 (m, 4H), 1.09 (t, 3H), 1.03 (d, 6H); m/z 197.

### Method 32

### N-{1-[1-Amino-meth-(Z)-ylidene]-2-oxo-propyl}-N-ethyl-isobutyramide

N-Ethyl-N-(5-methyl-isoxazol-4-yl)-isobutyramide (Method 31; 15.6 g, 0.08 mol) and 10% Pd on carbon (3.9 g) were added to EtOH and stirred at 4 atm over night. The reaction was filtered and solvent removed in vacuo to yield an off white solid. Ether (150 ml) was added and the reaction was sonicated for 10 minutes before being filtered and dried. A white solid was obtained (11 g, 69%). NMR (400.132 MHz) 7.57 (t, 1H), 6.99 (brs, 1H), 6.79 (brs, 1H), 3.39-3.31 (m, 3H), 2.43-2.33 (m, 1H), 2.09 (s, 3H), 0.92-0.81 (m, 9H); m/z 199.

### Method 33

### 1-(3-Ethyl-2-isopropyl-3H-imidazol-4-yl)-ethanone

N-{1-[1-Amino-meth-(Z)-ylidene]-2-oxo-propyl}-N-ethyl-isobutyramide (Method 32; 11 g, 0.056 mol) and NaOH (2.7 g, 0.067 mol) were added to EtOH (150 ml) and heated at reflux for 4 hours. To the reaction was added solid NH₄Cl (4.4 g, 0.084 mol) and this was stirred overnight. The resulting slurry was concentrated in vacuo, ether (200 ml) was added, the mixture was stirred for 10 minutes then filtered. The filtrate was concentrated in vacuo to yield orange oil. This was distilled using bulb-to-bulb distillation (0.76 mmbar/120° C.) to give a clear oil (8.2 g, 81%). NMR (400.132 MHz, CDCl₃) 7.74 (s, 1H), 4.34 (q, 2H), 3.04 (septet, 1H), 2.44 (s, 3H), 1.35 (d, 6H), 1.32 (t, 3H); m/z 181.

### Method 34

### (E)-3-Dimethylamino-1-(3-ethyl-2-isopropyl-3H-imidazol-4-yl)-propenone

1-(3-Ethyl-2-isopropyl-3H-imidazol-4-yl)-ethanone (Method 33; 7.0 g, 0.039 mol) and DMFDMA (13.3 ml, 0.078 mol) were added to DMF and heated at 130° C. for 6 hours. The solvent was removed in vacuo to yield a dark gum. Ether (50 ml) was added to the gum to afford a golden solid which was filtered and dried to give the title compound (7.7 g, 84%).

NMR (400.132 MHz, CDCl₃) 7.66 (d, 1H), 7.54 (s, 1H), 5.52 (d, 1H), 4.42 (q, 2H), 3.09-2.89 (m, 9H), 1.36-1.33 (m, 9H); m/z 236

### Method 35

### 4-(3-Ethyl-2-isopropyl-3H-imidazol-4-yl)-pyrimidin-2-ylamine

(E)-3-Dimethylamino-1-(3-ethyl-2-isopropyl-3H-imidazol-4-yl)-propenone (Method 34; 6.5 g, 0.028 mol) and guanidine carbonate (12.5 g, 0.069 mol) were added to butanol (100 ml) and heated at reflux for 5 days. The solvent was removed in vacuo to yield a yellow gum. Purification by column chromatography on silica using 0-5% MeOH in DCM gave the title compound as a yellow solid. DCM (5 ml) and ether (50 ml) were added and the suspension was filtered and dried to give the title compound as a white solid (5.0 g, 77%). NMR (400.132 MHz) 8.14 (d, 1H), 7.53 (s, 1H), 6.84 (d, 1H), 6.56 (brs, 2H), 4.54 (q, 2H), 3.13 (septet, 1H), 1.25-1.20 (m, 9H); m/z 232.

### Method 36

### Cyclopropanecarboxylic acid ethyl-(5-methyl-isoxazol-4-yl)-amide

Ethyl-(5-methyl-isoxazol-4-yl)-amine hydrochloride (15 g, 0.092 mol) was added to DCM (200 ml), to this was added TEA (32 ml, 0.23 mol) followed by the slow addition of cyclopropylcarbonylchloride (10.2 g, 0.10 mol). The reaction was stirred for 30 minutes before the removal of the solvent in vacuo. The residue was treated with water (150 ml), extracted with ether (3×150 ml), dried, and the solvent was removed in vacuo to yield a yellow oil (12.2 g, 69%) which was used without further purification.

### Method 37

### N-{1-[1-Amino-meth-(Z)-ylidene]-2-oxo-propyl}-N-ethyl-cyclopropylamide

Cyclopropanecarboxylic acid ethyl-(5-methyl-isoxazol-4-yl)-amide (Method 36; 12.2 g, 0.08 mol) and 10% Pd on carbon (3.0 g) were added to EtOH (300 ml) and stirred at 4 atm overnight. The reaction was filtered and the solvent was removed in vacuo to yield a off white solid. Ether (150 ml) was added, this was sonicated for 10 minutes before being filtered and dried to give a white solid. (9.2 g, 59%); m/z 197.

### Method 38

### 1-(3-Ethyl-2-cyclopropyl-3H-imidazol-4-yl)-ethanone

N-{1-[1-Amino-meth-(Z)-ylidene]-2-oxo-propyl}-N-ethyl-cyclopropylamide (Method 37; 9.2 g, 0.047 mol) and NaOH (2.3 g, 0.056 mol) were added to EtOH (150 ml) and heated at reflux for 4 hours. To the reaction was added solid NH₄Cl (4.4 g, 0.084 mol) and the reaction was stirred overnight. The resulting slurry was concentrated in vacuo, ether (200 ml) was added, the reaction was stirred for 10 minutes and filtered. The filtrate was removed in vacuo to yield an orange oil. This was distilled using bulb-to-bulb distillation (0.50 mbar/110° C.) to give a clear oil (5.0 g, 60%). NMR (400.132 MHz, CDCl₃) 7.64 (s, 1H), 4.48 (q, 2H), 2.42 (s, 3H), 1.87-1.80 (m, 1H), 1.37 (t, 3H), 1.13-1.08 (m, 2H), 1.08-1.02 (m, 2H); m/z 179.

### Method 39

### (E)-1-(2-Cyclopropyl-3-ethyl-3H-imidazol-4-yl)-3-dimethylamino-propenone

1-(3-Ethyl-2-cyclopropyl-3H-imidazol-4-yl)-ethanone (Method 38; 3.5 g, 0.020 mol) and DMFDMA (6.7 ml, 0.039 mol) were added to DMF (50 ml) and heated at 130° C. for 6 hours. The solvent was removed in vacuo to yield a yellow solid. DCM (3.0 ml) was added followed by ether (50 ml) the reaction was sonicated for 10 minutes and then filtered. A yellow solid was obtained (3.4 g; 72%). NMR (400.132 MHz, CDCl₃) 7.65 (d, 1H), 7.45 (s, 1H), 5.50 (d, 1H), 4.56 (q, 2H), 3.13-2.88 (m, 6H), 1.87-1.81 (m, 1H), 1.39 (t, 3H), 1.09-1.06 (m, 2H), 1.02-0.98 (m, 2H); m/z 234.

### Method 40

### 4-(3-Ethyl-2-cyclopropyl-3H-imidazol-4-yl)-pyrimidin-2-ylamine

(E)-1-(2-Cyclopropyl-3-ethyl-3H-imidazol-4-yl)-3-dimethylamino-propenone (Method 39; 3.4 g, 0.015 mol) and guanidine carbonate (6.6 g, 0.036 mol) were added to butanol (60 ml) and heated at reflux for 4 days. The solvent was removed in vacuo, water (50 ml) was added and the residue was extracted with DCM (3×75 ml), dried and the solvent was removed in vacuo to yield an off white solid. DCM was added, followed by ether, the resulting solid was filtered and dried to give a white solid (2.75 g, 83%). NMR (400.132 MHz, CDCl₃) 8.19 (d, 1H), 7.95 (s, 1H), 6.83 (d, 1H), 4.94 (brs, 2H), 4.64 (q, 2H), 1.90-1.84 (m, 1H), 1.41 (t, 3H), 1.11-1.07 (m, 2H), 1.05-0.99 (m, 2H); m/z 230.

### Method 41

### N-Ethyl-2,2,2-trifluoro-N-(5-methyl-isoxazol-4-yl)-acetamide

Ethyl-(5-methyl-isoxazol-4-yl)-amine hydrochloride (15 g, 0.092 mol) was dissolved in pyridine (100 ml). To this was added trifluoroacetic anhydride (16.9 ml, 0.12 mol) and the reaction was stirred overnight before removal of the solvent in vacuo. The residue obtained was quenched with saturated NH₄Cl (200 ml), extracted with ether (3×200 ml), dried and solvent removed in vacuo to yield a yellow oil (18 g, 88%). NMR (400.132 MHz, CDCl₃) 8.03 (s, 1H), 3.55 (q, 2H), 2.26 (s, 3H), 1.05 (t, 3H); m/z 223.

### Method 42

### N-{1-[1-Amino-meth-(Z)-ylidene]-2-oxo-propyl}-N-ethyl-2,2,2-trifluoro-acetamide

N-Ethyl-2,2,2-trifluoro-N-(5-methyl-isoxazol-4-yl)-acetamide (Method 41; 18.0 g, 0.081 mol) and 10% Pd on carbon (4.0 g) were reacted under a atmosphere of hydrogen at 4 atm for 3 days. The reaction was filtered and solvent removed in vacuo to yield an off white solid, DCM (30 ml) and ether (100 ml) were added. The reaction was stirred for 10 minutes, filtered and dried to give a white solid (11.6 g, 64%); m/z 225.

### Method 43

### 1-(3-Ethyl-2-trifluoromethyl-3H-imidazol-4-yl)-ethanone

N-{1-[1-Amino-meth-(Z)-ylidene]-2-oxo-propyl}-N-ethyl-2,2,2-trifluoro-acetamide (Method 42; 11.6 g, 0.051 mol) and potassium carbonate (14.4 g, 0.103 mol) were added to dioxane (180 ml) and heated at reflux for 2 hours. The reaction was cooled, filtered and solvent removed in vacuo to yield yellow oil. Purification by column chromatography on silica using 0-40% ether in iso-hexane gave the title compound as a clear oil (8.9 g, 85%).

NMR (400.132 MHz, CDCl₃) 7.79 (s, 1H), 4.50 (q, 2H), 2.54 (s, 3H), 1.40 (t, 3H); m/z 207.

### Method 44

### (E)-3-Dimethylamino-1-(3-ethyl-2-trifluoromethyl-3H-imidazol-4-yl)-propenone

1-(3-Ethyl-2-trifluoromethyl-3H-imidazol-4-yl)-ethanone (Method 43; 7.0 g, 0.034 mol) and DMFDMA. (11.6 ml, 0.068 mol) were added to DMF (90 ml) and heated at 130° C. for 1 hour. The solvent was removed in vacuo to yield a yellow solid. Purification by column chromatography on silica using 0-5% MeOH in DCM gave the title product as a yellow solid. Ether was added followed by iso-hexane, the solid obtained filtered and dried to give the title compound. (7.6 g, 85%). NMR (400.132 MHz, CDCl₃) 7.74 (d, 1H), 7.55 (s, 1H), 5.53 (d, 1H), 4.57 (q, 2H), 3.17 (brs, 3H), 2.93 (brs, 3H), 1.42 (t, 3H); m/z 262.

### Method 45

### 4-(3-Ethyl-2-trifluoromethyl-3H-imidazol-4-yl)-pyrimidin-2-ylamine

(E)-3-Dimethylamino-1-(3-ethyl-2-trifluoromethyl-3H-imidazol-4-yl)-propenone (Method 44; 6.0 g, 0.023 mol) and guanidine carbonate (8.3 g, 0.046 mol) were added to 2-methoxyethoxy ether (80 ml) and heated at 140° C. for 2 days. The reaction was cooled and the solvent was removed in vacuo to yield a yellow solid. Water (100 ml) was added and the system was extracted with DCM (3×100 ml), dried and the solvent removed in vacuo to yield a yellow solid. Purification by column chromatography on silica using 0-5% MeOH in DCM gave the title compound as a yellow solid. Ether (20 ml) followed by iso-hexane (50 ml) were added to yield an off white solid which was filtered and dried (5.9 g, 100%); m/z 258.

### Method 46

### N-Ethyl-2,2-difluoro-N-(5-methyl-isoxazol-4-yl)-acetamide

Ethyl-(5-methyl-isoxazol-4-yl)-amine hydrochloride (15 g, 0.092 mol) and TEA were added to DCM (300 ml), this was cooled to 0° C. before the slow addition of difluoroacetyl chloride (11.5 g, 0.10 mol). The reaction was stirred for 1 hour before the removal of the solvent in vacuo. The obtained residue was quenched with saturated NH₄Cl (200 ml), extracted with ether (3×200 ml), dried and solvent removed in vacuo to yield a yellow oil (9.0 g, 48%). M/z 203 (M-H)⁻.

### Method 47

### N-{1-[1-Amino-meth-(Z)-ylidene]-2-oxo-propyl}-N-ethyl-2,2-difluoro-acetamide

N-Ethyl-2,2-difluoro-N-(5-methyl-isoxazol-4-yl)-acetamide (Method 46; 9.0 g, 0.044 mol) was treated with 10% palladium on carbon (3.0 g) under 4 atm of pressure. The reaction was filtered and solvent removed in vacuo, DCM was added and the reaction was filtered to yield an off white solid (3.0 g, 33%); m/z 207.

### Method 48

### 1-(2-Difluoromethyl-3-ethyl-3H-imidazol-4-yl)-ethanone

N-{1-[1-Amino-meth-(Z)-ylidene]-2-oxo-propyl}-N-ethyl-2,2-difluoro-acetamide (Method 47; 3.0 g, 0.014 mol) and potassium carbonate (3.9 g, 0.028 mol) were added to dioxane (50 ml) and heated at reflux overnight. The reaction was filtered and the solvent removed in vacuo to yield a yellow oil. Purification by column chromatography on silica using ether as eluent gave the title compound as a yellow solid (2.4 g, 92%). NMR (400.132 MHz, CDCl₃) 7.74 (s, 1H), 6.78 (t, 1H), 4.54 (q, 2H), 2.51 (s, 3H), 1.40 (t, 3H); m/z 189.

### Method 49

### (E)-1-(2-Difluoromethyl-3-ethyl-3H-imidazol-4-yl)-3-dimethylamino-propenone

1-(2-Difluoromethyl-3-ethyl-3H-imidazol-4-yl)-ethanone (Method 48; 2.4 g, 0.013 mol) and DMFDMA (4.4 ml, 0.026 mol) were added to DMF (50 ml) and heated at 130° C. for 20 minutes. The solvent was removed in vacuo to yield a yellow solid. DCM (3.0 ml) was added followed by ether (50 ml), sonicated for 10 minutes and then filtered. A yellow solid was obtained (2.7 g, 85%). NMR (400.132 MHz, CDCl₃) 7.71 (d, 1H), 7.52 (s, 1H), 6.75 (t, 1H), 5.52 (d, 1H), 4.61 (q, 2H), 3.19-2.88 (m, 6H), 1.42 (t, 3H); m/z 244.

### Method 50

### 4-(2-Difluoromethyl-3-ethyl-3H-imidazol-4-yl)-pyrimidin-2-Ylamine

(E)-1-(2-Difluoromethyl-3-ethyl-3H-imidazol-4-yl)-3-dimethylamino-propenone (Method 49; 2.7 g, 0.011 mol) and guanidine carbonate (4.0 g, 0.022 mol) were added to ethylene glycol diethyl ether (30 ml) and heated at 137° C. for 2 days. The solvent was removed in vacuo to yield a yellow solid. DCM (5.0 ml) was added followed by ether (50 ml), the obtained solid was filtered and dried. A white solid was obtained (2.5 g, 96%). NMR (400.132 MHz) 8.27 (d, 1H), 7.72 (s, 1H), 7.23 (t, 1H), 6.97 (d, 1H), 6.71 (s, 2H), 4.70 (q, 2H), 1.30 (t, 3H); m/z 240.

### Method 51

### N-[(Z)-1-Acetyl-2-aminovinyl]-N-isopropylcyclopropanecarboxamide

Cyclopropanecarboxylic acid isopropyl-(5-methyl-isoxazol-4-yl)-amide (Method 36 in WO03/076434; 18 g, 0.086 mol) and 10% palladium on carbon (3.0 g) in EtOH were reacted with hydrogen at 4 atm of pressure. The reaction was filtered and solvent removed in vacuo to yield a solid, ether was added and the solid was filtered (7.9 g, 44%); m/z 211.

### Method 52

### 1-(2-Cyclopropyl-3-isopropyl-3H-imidazol-4-yl)-ethanone

N-[(Z)-1-Acetyl-2-aminovinyl]-N-isopropylcyclopropanecarboxamide (Method 51; 7.9 g, 0.038 mol) and sodium hydroxide (2.28 g, 0.057 mol) were added to EtOH (150 ml) and heated at reflux overnight. The solvent was removed in vacuo and the resulting solid was treated with saturated NH₄Cl (100 ml), extracted with ether (3×1100 ml), dried and solvent removed in vacuo to yield a black oil. Purification by column chromatography on silica using 100% ether gave the title compound as a yellow oil (3.9 g, 53%). NMR (400.132 MHz, CDCl₃) 7.65 (s, 1H), 5.63-5.48 (m, 1H), 2.44 (s, 3H), 1.98-1.91 (m, 1H), 1.57 (d, 6H), 1.17-1.11 (m, 2H), 1.07-1.03 (m, 2H); m/z 193.

### Method 53

### (E)-1-(2-Cyclopropyl-3-isopropyl-3H-imidazol-4-yl)-3-dimethylamino-propenone

1-(2-Cyclopropyl-3-isopropyl-3H-imidazol-4-yl)-ethanone (Method 52; 3.74 g, 0.019 mol) and DMFDMA (6.66 ml, 0.039 mol) were added to DMF and heated at 130° C. for 4 hours. The solvent was removed in vacuo to yield an orange gum, DCM was added followed by ether to give the title compound as a yellow solid which was filtered and dried (4.5 g, 96%). NMR (400.132 MHz, CDCl₃) 7.63 (d, 1H), 7.40 (s, 1H), 5.61 (septet, 1H), 5.50 (d, 1H), 3.12-2.88 (m, 6H), 1.98-1.92 (m, 1H), 1.60 (d, 6H), 1.13-1.09 (m, 2H), 1.03-0.98 (m, 2H); m/z 248.

### Method 54

### 4-(2-Cyclopropyl-3-isopropyl-3H-imidazol-4-yl)-pyrimidin-2-ylamine

(E)-1-(2-Cyclopropyl-3-isopropyl-3H-imidazol-4-yl)-3-dimethylamino-propenone (Method 53; 4.5 g, 0.019 mol) and guanidine carbonate (6.55 g, 0.036 mol) were added to ethylene glycol diethyl ether (75 ml) and heated at 142° C. for 2 days. The solvent was removed in vacuo, water (100 ml) was added then extracted with DCM (3×150 ml), dried and the solvent removed in vacuo to yield a yellow solid. DCM was added followed by ether, the mixture was stirred for 30 minutes before being filtered and dried (3.6 g, 78%). NMR (400.132 MHz, CDCl₃) 8.22 (d, 1H), 7.28 (s, 1H), 6.79 (d, 1H), 5.57 (septet, 1H), 5.01 (brs, 2H), 2.03-1.96 (m, 1H), 1.64 (d, 6H), 1.17-1.13 (m, 2H), 1.05-1.00 (m, 2H); m/z 244.

### Method 55

### Ethyl 4-{[4-(1-isopropyl-2-methyl-1H-imidazol-5-yl)pyrimidin-2-yl]amino}benzoate

To a solution of 4-(3-isopropyl-2-methyl-3H-imidazol-4-yl)-pyrimidin-2-ylamine (Method 18; 7.8 g) in dioxane (200 ml) was added ethyl 4-iodobenzoate (9.445 g), palladium (II) acetate (461 mg), XANTPHOS (1.785 g), and caesium carbonate (22.29 g). The mixture was degassed, and purged with nitrogen, then heated under reflux for 3 hours. The mixture was cooled to room temperature, the solids were removed by filtration, then the filtrate concentrated in vacuo. Purification on silica using 2-5% MeOH in DCM as eluent gave the title compound as a yellow solid (3.82 g, 31%). NMR 9.87 (s, 1H), 8.46 (d, 1H), 7.90-7.83 (m, 4H), 7.45 (s, 1H), 7.14 (d, 1H), 5.72-5.63 (m, 1H), 4.27 (q, 2H), 2.49 (s, 3H), 1.47 (d, 6H), 1.30 (t, 3H); m/z 366.

### Method 56

### 4-{[4-(1-Isopropyl-2-methyl-1H-imidazol-5-yl)pyrimidin-2-yl]amino}benzoic acid sodium salt

Ethyl 4-{[4-(1-isopropyl-2-methyl-1H-imidazol-5-yl)pyrimidin-2-yl]amino}benzoate (Method 55; 3.82 g) was dissolved in THF (130 ml) then a solution of NaOH (419 mg) in water (20 ml) was added. The mixture was heated under reflux for 2 days. The mixture was concentrated in vacuo, then dissolved in water (400 ml) and washed with EtOAc (2×300 ml). The aqueous layer was concentrated in vacuo to yield the title compound as a white solid (3.53 g, 94%), NMR 9.43 (s, 1H), 8.38 (d, 1H), 7.79 (d, 2H), 7.56 (d, 2H), 7.41 (s, 1H), 7.03 (d, 1H), 5.82-5.72 (m, 1H), 2.49 (s, 3H), 1.44 (d, 6H); m/z 338.

### Method 57

### Ethyl 4-{[5-fluoro-4-(1-isopropyl-2-methyl-1H-imidazol-5-yl)pyrimidin-2-yl]amino}benzoate

To a solution of 5-fluoro-4-(3-isopropyl-2-methyl-3H-imidazol-4-yl)-pyrimidin-2-ylamine (Method 17; 5.32 g) in dioxane (100 ml) was added ethyl 4-iodobenzoate (3.59 g), palladium (II) acetate (305 mg), XANTPHOS (1.18 g), and caesium carbonate (14.74 g). The mixture was degassed, and purged with nitrogen, then heated under reflux for 3 hours. The mixture was cooled to room temperature, the solids were removed by filtration, then the filtrate concentrated in vacuo. Purification on silica using 2-5% MeOH in DCM as eluent gave the title compound as a yellow solid (2.75 g, 32%). NMR 9.97 (s, 1H), 8.62 (d, 1H), 7.88 (d, 2H), 7.80 (d, 2H), 7.38 (d, 1H), 5.47-5.38 (m, 1H), 4.27 (q, 2H), 2.53 (s, 3H), 1.46 (d, 6H), 1.30 (t, 3H); m/z 384.

### Method 58

### 4-{[5-Fluoro-4-(1-isopropyl-2-methyl-1H-imidazol-5-yl)pyrimidin-2-yl]amino}benzoic acid lithium salt

To a stirred solution of ethyl 4-{[5-fluoro-4-(1-isopropyl-2-methyl-1H-imidazol-5-yl)pyrimidin-2-yl]amino}benzoate (Method 57; 2.75 g) in EtOH (70 ml) was added a solution of lithium hydroxide (301 mg) in water (15 ml). The mixture was heated under reflux for 18 hours, then concentrated in vacuo and partitioned between water (300 ml) and EtOAc (300 ml). The aqueous layer was washed with further EtOAc (200 ml) then concentrated in vacuo to yield the title compound as a white solid (2.07 g, 80%). NMR 9.56 (s, 1H), 8.53 (d, 1H), 7.80 (d, 2H), 7.53 (d, 2H), 7.36 (d, 1H), 5.56-5.46 (m, 1H), 2.51 (s, 3H), 1.43 (d, 6H); m/z 356.

### Method 59

### 4-Bromo-2-fluoro-N-(1-methylpiperidin-4-yl)benzamide

4-Bromo-2-fluorobenzoic acid (5.0 g, 0.023 mol) and HBTU (9.5 g, 0.025 mol) were dissolved in DMF (75 ml). To this was added the methylpiperidin-4-amine (2.9 g, 0.025 mol) followed by DIPEA (8.8 ml, 0.051 mmol). The reaction was stirred for 90 minutes and the DMF removed in vacuo. The resultant solid was quenched with 2.0M NaOH (50 ml) and extracted with DCM (3×100 ml). The organic phase was dried and the solvent removed in vacuo to yield a brown sludge which solidified on cooling. This solid was dissolved in DCM and ether added until a solid precipitated. The title compound was then filtered off and dried (7.3 g, 100%). NMR 8.47 (d, 1H), 7.65 (d, 1H), 7.54-7.47 (m, 2H), 4.06-3.91 (m, 1H), 3.38-3.34 (m, 2H), 3.09 (t, 2H), 2.74 (s, 3H), 2.04-1.98 (m, 2H), 1.74 (q, 2H); m/z 316.

### Method 60

### 4-[1-Isopropyl-2-(methoxymethyl)-1H-imidazol-4-yl]-pyrimidin-2-ylamine

The title compound was prepared by the procedure of Method 17 and on the same scale, using guanidine carbonate and 3-(dimethylamino)-1-[1-isopropyl-2-(methoxymethyl)-1H-imidazol-5-yl]prop-2-en-1-one (Method 50 of WO 03/076434). NMR (400.132 MHz, CDCl₃): 8.26 (d, 1H), 7.38 (s, 1H), 6.82 (d, 1H), 5.30 (septet, 1H), 5.14 (s, 2H), 4.64 (s, 3H), 3.39 (s, 3H), 1.59 (d, 6H); m/z 248.

### Method 61

### tert-Butyl 3-[(4-iodobenzoyl)amino]piperidine-1-carboxylate

tert-Butyl 3-aminopiperidine-1-carboxylate (1.0 g, 5.0 mmol) and triethylamine (1.4 ml, 10.0 mmol) were stirred in THF (10 ml) under an inert atmosphere. 4-iodobenzoyl chloride (1.33 g, 5.0 mmol) was added portionwise over 1 minute. Stirring was continued for a further 2 hours. The solvent was evaporated in vacuo and the residue partitioned between EtOAc (25 ml) and 1M NaOH (10 ml). The organics were washed with water (10 ml) and brine (10 ml), dried and evaporated to afford the title compound as a white solid (1.7 g, 4.0 mmol, 80%). NMR 8.26 (d, 1H), 7.83 (d, 2H), 7.61 (d, 2H), 4.00-3.65 (m, 3H), 2.79 (app t, 2H), 1.95-1.63 (m, 2H), 1.59-1.37 (m, 1H); m/z 431.

### Method 62

### 4-Bromo-3-fluorobenzoic acid

4-Bromo-3-fluorotoluene (24.4 g, 0.128 mol) was added to a mixture of KMnO4 (24 g, 0.154 mol) in water (150 ml). The mixture was heated at 95° C. for 2 hrs then additional KMnO4 (24 g) was added, after a further 2 hrs at 95° C. additional KMnO4 (24 g) was added and heating was maintained at 95° C. for 18 hours. The hot mixture was then filtered through a pad of diatomaceous earth and washed with water. The filtrate was acidified to pH 2 with 2N HCl and the white suspension was filtered and dried to give the product (7.33 g). The filtrate was extracted with EtOAc (2×250 ml), the organic extracts dried and evaporated in vacuo to give additional product. Combined product (7.92 g, 28%). NMR: 7.68 (d, 1H), 7.74 (d, 1H), 7.82-7.87 (m, 1H); M/z (M-H)⁻ 217.

### Method 63

### 4-Bromo-3-fluoro-N,N-dimethylbenzamide

Oxalyl chloride (7.0 ml, 79.92 mmol) was added to a suspension of 4-bromo-3-fluorobenzoic acid (Method 62; 7.92 g, 36.33 mmol) in DCM (250 ml) containing catalytic DMF. The mixture stirred for 18 hours then concentrated in vacuo. The residue was dissolved in DCM (250 ml) and dimethylamine (5.6M in EtOH) (17 ml) was added and the mixture stirred at ambient temperature for 2 hours. The reaction mixture was washed with NaHCO₃ (3×150 ml), brine (150 ml), dried and concentrated in vacuo to give the title compound (5.01 g, 56%); NMR 7.56-7.62 (t, 1H), 7.19 (d, 1H), 7.09 (d, 1H), 3.09 (s, 3H), 2.99 (s, 3H); M/z 248.

### Example 98

The following illustrate representative pharmaceutical dosage forms containing the compound of formula (I), or a pharmaceutically acceptable salt or in vivo hydrolysable ester thereof (hereafter compound X), for therapeutic or prophylactic use in humans:—

| | mg/tablet |
|---|---|
| (a): Tablet I | |
| Compound X | 100 |
| Lactose Ph. Eur | 182.75 |
| Croscarmellose sodium | 12.0 |
| Maize starch paste (5% w/v paste) | 2.25 |
| Magnesium stearate | 3.0 |
| (b): Tablet II | |
| Compound X | 50 |
| Lactose Ph. Eur | 223.75 |
| Croscarmellose sodium | 6.0 |
| Maize starch | 15.0 |
| Polyvinylpyrrolidone (5% w/v paste) | 2.25 |
| Magnesium stearate | 3.0 |
| (c): Tablet III | |
| Compound X | 1.0 |
| Lactose Ph. Eur | 93.25 |
| Croscarmellose sodium | 4.0 |
| Maize starch paste (5% w/v paste) | 0.75 |
| Magnesium stearate | 1.0 |

| (d): Capsule | mg/capsule |
|---|---|
| Compound X | 10 |
| Lactose Ph. Eur | 488.5 |
| Magnesium stearate | 1.5 |

| (e): Injection I | (50 mg/ml) | |
|---|---|---|
| Compound X | 5.0% | w/v |
| 1M Sodium hydroxide solution | 15.0% | v/v |
| 0.1M Hydrochloric acid | (to adjust pH to 7.6) | |
| Polyethylene glycol 400 | 4.5% | w/v |
| Water for injection | to 100% | |

| (f): Injection II | 10 mg/ml | |
|---|---|---|
| Compound X | 1.0% | w/v |
| Sodium phosphate BP | 3.6% | w/v |
| 0.1M Sodium hydroxide solution | 15.0% | v/v |
| Water for injection | to 100% | |

| (g): Injection III | (1 mg/ml, buffered to pH6) | |
|---|---|---|
| Compound X | 0.1% | w/v |
| Sodium phosphate BP | 2.26% | w/v |
| Citric acid | 0.38% | w/v |
| Polyethylene glycol 400 | 3.5% | w/v |
| Water for injection | to 100% | |

Note
The above formulations may be obtained by conventional procedures well known in the pharmaceutical art. The tablets (a)-(c) may be enteric coated by conventional means, for example to provide a coating of cellulose acetate phthalate.

1: A compound of formula (I): 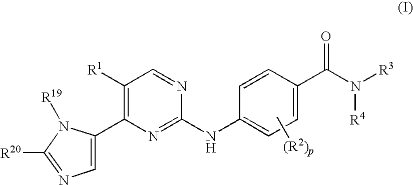
wherein:
R¹ is hydrogen or halo;
R² is halo, nitro, cyano, hydroxy, amino, carboxy, carbamoyl, mercapto, methylthio, mesyl, trifluoromethyl, trifluoromethoxy, C₁₋₆alkyl, C₁₋₆alkoxy, C₂₋₆alkenyl or C₂₋₆alkynyl;
p is 0-4; wherein the values of R² may be the same or different;
R³ and R⁴ are independently selected from hydrogen, C₁₋₆alkyl, C₂₋₆alkenyl, C₂₋₆alkynyl, carbocyclyl or heterocyclyl; wherein R³ and R⁴ may be independently optionally substituted on carbon by one or more R⁵; and wherein if said heterocyclyl contains an —NH— moiety that nitrogen may be optionally substituted by a group selected from R⁶;
R¹⁹ is selected from ethyl, propyl, isopropyl, butyl, iso-butyl, sec-butyl, τ-butyl, cyclopropyl, cyclopropylmethyl, 1-cyclopropylethyl or cyclobutyl; wherein R¹ may be optionally substituted on carbon by one or more R²¹;
R²⁰ is methyl, ethyl, isopropyl, fluoromethyl, difluoromethyl, trifluoromethyl, methoxymethyl, cyclopropylmethyl or cyclopropyl;
R⁵ is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl, heterocyclyl, carbocyclylC₁₋₆alkyl-R⁷—, heterocyclylC₁₋₆alkyl-R⁸—, carbocyclyl-R⁹— or heterocyclyl-R¹⁰—; wherein R⁵ may be optionally substituted on carbon by one or more R¹¹; and wherein if said heterocyclyl contains an —NH— moiety that nitrogen may be optionally substituted by a group selected from R¹²;
R⁶ and R¹² are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl; wherein R⁶ and R¹² may be independently optionally substituted on carbon by one or more R¹³.
R⁷, R⁸, R⁹ and R¹⁰ are independently selected from —O—, —N(R¹⁴)—, —C(O)—, —N(R¹⁵)C(O)—, —C(O)N(R¹⁶)—, —S(O)ₛ—, —SO₂N(R¹⁷)— or —N(R¹⁸)SO₂—; wherein R⁴, R¹⁵, R¹⁶, R¹⁷ and R¹⁸ are independently selected from hydrogen or C₁₋₆alkyl and s is 0-2;
R¹¹ and R¹³ are independently selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl; and
R²¹ is selected from halo, methoxy and hydroxy;
or a pharmaceutically acceptable salt or an in vivo hydrolysable ester thereof.

2: The compound of formula (I), or a pharmaceutically acceptable salt or an in vivo hydrolysable ester thereof, as claimed in claim 1, wherein R¹ is hydrogen or fluoro.

3: The compound of formula (I), or a pharmaceutically acceptable salt or an in vivo hydrolysable ester thereof, as claimed in claim 1, wherein R² is halo, cyano or C₁₋₆alkyl.

4: The compound of formula (I), or a pharmaceutically acceptable salt or an in vivo hydrolysable ester thereof, as claimed in claim 1, wherein p is 0 or 1.

5: The compound of formula (I), or a pharmaceutically acceptable salt or an in vivo hydrolysable ester thereof, as claimed in claim 1, wherein R³ and R⁴ are independently selected from hydrogen, C₁₋₆alkyl, carbocyclyl or heterocyclyl; wherein R³ and R⁴ may be independently optionally substituted on carbon by one or more R⁵; and wherein if said heterocyclyl contains an —NH— moiety that nitrogen may be optionally substituted by a group selected from R⁶; wherein
R⁵ is selected from hydroxy, N,N—(C₁₋₆alkyl)₂-amino and heterocyclyl;
R⁶ is selected from C₁₋₆alkyl and C₁₋₆alkoxycarbonyl; wherein R⁶ may be independently optionally substituted on carbon by one or more R¹³;
R¹³ is methoxy.

6: The compound of formula (I), or a pharmaceutically acceptable salt or an in vivo hydrolysable ester thereof, as claimed in claim 1, wherein R¹⁹ is selected from ethyl, isopropyl, cyclopropylmethyl, 1-cyclopropylethyl or cyclobutyl.

7: The compound of formula (I), or a pharmaceutically acceptable salt or an in vivo hydrolysable ester thereof, as claimed in claim 1, wherein R²⁰ is methyl, ethyl, isopropyl, difluoromethyl, trifluoromethyl, methoxymethyl or cyclopropyl.

8: The compound of formula (I) as claimed in claim 1: 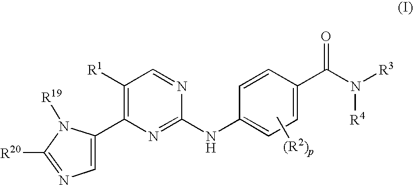
wherein:
R¹ is hydrogen or fluoro;
R² is fluoro, chloro, cyano or methyl;
p is 0 or 1;
R³ and R⁴ are independently selected from hydrogen, methyl, cyclopropyl, 2-hydroxyethyl, 1-methylpiperidin-4-yl, piperidin-3-yl, tetrahydropyran-4-yl, 1,1-dioxidotetrahydrothien-3-yl, 2-dimethylaminoethyl, 1-methyl-2-dimethylaminoethyl, piperidin-1-ylethyl, 2-morpholinoethyl, 1-(2-methoxyethyl)piperidin-4-yl, 2-thiomorpholinoethyl, 2-pyrrolidin-1-ylethyl and 1-(t-butoxycarbonyl)piperidin-3-yl;
R¹⁹ is selected from ethyl, isopropyl, cyclopropylmethyl, 1-cyclopropylethyl or cyclobutyl;
R²⁰ is methyl, ethyl, isopropyl, difluoromethyl, trifluoromethyl, methoxymethyl or cyclopropyl;
or a pharmaceutically acceptable salt or an in vivo hydrolysable ester thereof.

9: The compound of formula (I) as claimed in claim 1: 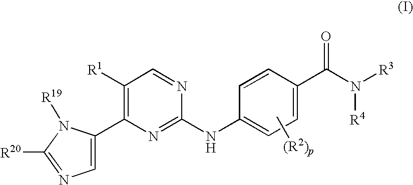
selected from:
2-fluoro-4-{[4-(1-isopropyl-2-methyl-1H-imidazol-5-yl)pyrimidin-2-yl]amino}-N-methylbenzamide;
4-{[4-(1-isopropyl-2-methyl-1H-imidazol-5-yl)pyrimidin-2-yl]amino}-N-methylbenzamide;
4-{[4-(1-isopropyl-2-methyl-1H-imidazol-5-yl)pyrimidin-2-yl]amino}-N,N-dimethylbenzamide;
4-{[5-fluoro-4-(1-isopropyl-2-methyl-1H-imidazol-5-yl)pyrimidin-2-yl]amino}-N-methylbenzamide;
[4-(3-isopropyl-2-methyl-3H-imidazol-4-yl)-pyrimidin-2-ylamino]-benzamide;
4-{[5-fluoro-4-(1-isopropyl-2-methyl-1H-imidazol-5-yl)pyrimidin-2-yl]amino}-N-(1-methylpiperidin-4-yl)benzamide;
4-{[4-(1-isopropyl-2-methyl-1H-imidazol-5-yl)pyrimidin-2-yl]amino}-N-(2-piperidin-1-ylethyl)benzamide;
4-{[4-(1-isopropyl-2-methyl-1H-imidazol-5-yl)pyrimidin-2-yl]amino}-N-(2-morpholin-4-ylethyl)benzamide;
4-{[4-(1-isopropyl-2-methyl-1H-imidazol-5-yl)pyrimidin-2-yl]amino}-N-[1-(2-methoxyethyl)piperidin-4-yl]benzamide; and
2-fluoro-4-{[5-fluoro-4-(1-isopropyl-2-methyl-1H-imidazol-5-yl)pyrimidin-2-yl]amino}-N-(1-methylpiperidin-4-yl)benzamide;
or a pharmaceutically acceptable salt or an in vivo hydrolysable ester thereof.

10: A process for preparing a compound of formula (I), as claimed in claim 1, or a pharmaceutically acceptable salt or an in vivo hydrolysable ester thereof, which process comprises of:
Process a) reaction of a pyrimidine of formula (II): 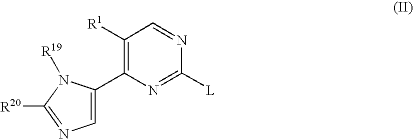
wherein L is a displaceable group; with an aniline of formula (III): 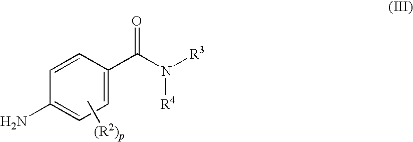
or
Process b) reacting a compound of formula (IV): 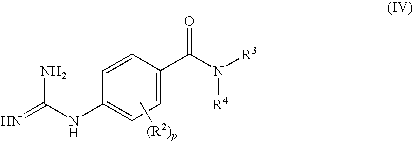
with a compound of formula (V): 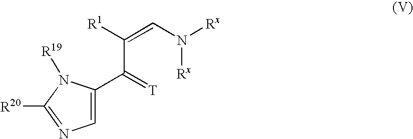
wherein T is O or S; R^{x} may be the same or different and is selected from C₁₋₆alkyl; or
Process c) reacting an acid of formula (VI): 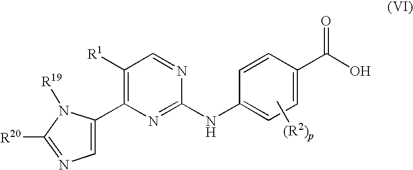
or an activated derivative thereof; with an amine of formula (VII):
HNR³R⁴ (VII)
or
Process d) for compounds of formula (I); reacting a pyrimidine of formula (VIII) 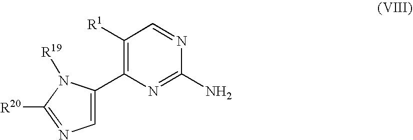
with a compound of formula (IX): 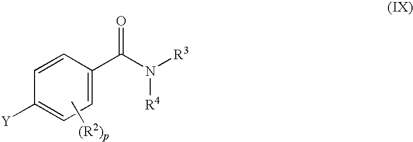
where Y is a displaceable group;
and thereafter optionally
i) converting a compound of the formula (I) into another compound of the formula (I);
ii) removing any protecting groups;
iii) forming a pharmaceutically acceptable salt or in vivo hydrolysable ester.

11: A pharmaceutical composition which comprises a compound of the formula (I), or a pharmaceutically acceptable salt or in vivo hydrolysable ester thereof, as claimed in any one of claims 1-9, and a pharmaceutically-acceptable diluent or carrier.

12-17. (canceled)

18: A method of producing an anti-cell-proliferation effect, in a warm-blooded animal in need of such treatment, which comprises administering to said animal an effective amount of a compound of formula (I) or a pharmaceutically acceptable salt or in vivo hydrolysable ester thereof, as claimed in claim 1.

19: A method of producing a CDDK2 inhibitory effect, in a warm-blooded animal in need of such treatment, which comprises administering to said animal an effective amount of a compound of formula (I) or a pharmaceutically acceptable salt or in vivo hydrolysable ester thereof, as claimed in claim 1.

20: A method of treating cancer, in a warm-blooded animal in need of such treatment, which comprises administering to said animal an effective amount of a compound of formula (I) or a pharmaceutically acceptable salt or in vivo hydrolysable ester thereof, as claimed in claim 1.

21: A method of treating leukaemia or lymphoid malignancies or cancer of the breast, lung, colon, rectum, stomach, liver, kidney, prostate, bladder, pancreas, vulva, skin or ovary, in a warm-blooded animal in need of such treatment, which comprises administering to said animal an effective amount of a compound of formula (I) or a pharmaceutically acceptable salt or in vivo hydrolysable ester thereof, as claimed in claim 1.

22: A method of treating cancer, fibroproliferative disorders, differentiative disorders, psoriasis, rheumatoid arthritis, Kaposi's sarcoma, haemangioma, acute or chronic nephropathies, atheroma, atherosclerosis, arterial restenosis, autoimmune diseases, acute or chronic inflammation, bone diseases or ocular diseases with retinal vessel proliferation, in a warm-blooded animal in need of such treatment, which comprises administering to said animal an effective amount of a compound of formula (I) or a pharmaceutically acceptable salt or in vivo hydrolysable ester thereof, as claim 1.
